(19) United States
(12) Patent Application Publication
(10) Pub. No.: US 2023/0416314 A1
(43) Pub. Date: Dec. 28, 2023

(54) MODIFIED HIGH MOLECULAR WEIGHT GLUTENIN SUBUNIT AND USES THEREOF

(71) Applicant: Ukko Inc., San Francisco, CA (US)

(72) Inventors: Yanay OFRAN, Tel Aviv (IL); Assaf BIRAN, Tel Aviv (IL); Shiri ZAKIN, Sde Warburg (IL); Moshe BEN-DAVID, Rehovot (IL); Anna CHUPRIN, Tel Aviv (IL); Orly MARCU GARBER, Modiin (IL)

(73) Assignee: Ukko Inc., San Francisco, CA (US)

(21) Appl. No.: 18/035,751

(22) PCT Filed: Nov. 24, 2021
(86) PCT No.: PCT/US2021/060672

§ 371 Date: May 8, 2023

Related U.S. Application Data
(60) Provisional application No. 63/221,565, filed on Jul. 14, 2021.
(60) Provisional application No. 63/192,639, filed on May 25, 2021.
(60) Provisional application No. 63/118,676, filed on Nov. 26, 2020.

(51) Int. Cl. C07K 14/415; C12N 15/82; A23L 33/18; A01H 6/46

(52) U.S. Cl. CPC C07K 14/415; A01H 6/4684; A23L 33/18; C12N 15/8251

(57) ABSTRACT

A de-epitoped high molecular weight (HMW) glutenin is provided. Methods of generating same, and uses thereof are also provided.

### SEQUENCE LISTING STATEMENT

The instant application contains a Sequence Listing which has been submitted electronically in ASCII format and is hereby incorporated by reference in its entirety. Said ASCII copy, created on Nov. 15, 2021, is named P-608944-PC_SL.txt and is 334,880 bytes in size.

### FIELD OF INVENTION

The disclosure relates in general to methods of de-epitoping high molecular weight glutenin (HMW glutenin) and uses thereof for the management of gluten sensitivity, including for example in subjects suffering from celiac disease.

### BACKGROUND

Celiac disease (CD) is an acquired chronic immune disorder that develops in susceptible individuals, many of whom are of HLA genotype DQ2 or DQ8, wherein a minority of subjects do not have either DQ2 or DQ8 but are predominantly of genotype DQ7.5. The disorder is related to an environmental factor, gluten, which is a group of seed storage proteins of wheat and related grains like rye and barley. HMW glutenin is one of these seed storage proteins. The prevalence of CD in Europe and in the United States has been estimated to be approximately 1-2% of the population. CD has a wide range of clinical manifestations including latent or silent celiac disease, disease with only mild gastrointestinal disturbances, chronic gastrointestinal symptoms, malabsorption, and/or weight loss. CD is often diagnosed in patients with isolated iron deficiency anemia.

Some CD relevant T-cell epitopes from wheat, barley, and rye are known in the art, see for example Shewry P R, Tatham A S. Improving wheat to remove coeliac epitopes but retain functionality. J Cereal Sci. 2016; 67:12-21.

The ingestion of gluten-containing cereals can also induce manifestations outside the gut, such as osteoporosis, peripheral and central nervous system involvement, mild or severe liver disease, infertility problems, and the classical example is the gluten-induced skin disease, dermatitis herpetiformis.

For patients with CD, lifelong complete gluten exclusion needs to be strictly followed to avoid a substantially enhanced risk for the development of further complications, such as bone disorders, infertility, and cancer. The mortality rate in patients with CD exceeds that of the general population; however, there is a trend towards reduction in mortality after 1-5 years on a gluten-free diet.

However, following a completely gluten-free diet is very challenging. Even highly motivated patients who try to maintain a strict dietary regimen are affected due to inadvertent or background exposure to gluten. As many as 80% of patients with CD who are in clinical remission and who claim to be following a gluten-free diet, have persistent abnormalities in small bowel biopsy specimens. Inadvertent exposure to gluten has been identified as the leading cause of non-responsive CD among clinically diagnosed patients who were presumed to be on a gluten-free diet.

Taken together, there is an acute need for additional dietary therapies for subject suffering from CD, which are both non-costly and accessible.

### SUMMARY

In one aspect, disclosed herein are de-epitoped high molecular weight (HMW) glutenin proteins comprising a first mutation at position 1 and a second mutation at position 9 of a repeating antigenic unit, wherein the amino acid sequence of said non-mutated repeating antigenic unit is set forth in any of SEQ ID NOs: 1-33, 35, or 71-99. In a related aspect, the amino acid sequence of the non-de-epitoped HMW glutenin is set forth in any of SEQ ID NOs: 43, 44, 45, 46, 47, 48, 152, 153, or 154, or is set forth in an amino acid sequence having at least 50% identity with the sequences set forth in any of SEQ ID NOs: 43, 44, 45, 46, 47, 48, 152, 153, or 154.

In a further related aspect, the first mutation, or said second mutation, or both comprises a substitution mutation. In another further related aspect, the first mutation, or said second mutation, or both comprises a deletion mutation. In yet another related aspect, the mutation comprises a substitution mutation replacing the amino acid residue at position 9 of said repeating antigenic unit with a positively charged amino acid or a small amino acid; or a glutamate; or a combination thereof when more than one repeating antigenic unit is mutated. In certain aspects the positively charged amino acid is arginine or histidine; or the small amino acid is serine or threonine; or a combination thereof when more than one repeating antigenic unit is mutated.

In another further related aspect, the mutation at position 1 of said repeating antigenic unit comprises a substitution mutation that replaces the amino acid residue at position 1 of said repeating antigenic unit with a histidine, a proline, a serine, an alanine, a lysine, or a glutamate.

In certain related aspects, the substituting at position 1 of the antigenic unit is a replacement with a histidine, and the substituting at position 9 of the antigenic unit is a replacement with histidine; the substituting at position 1 of the antigenic unit is a replacement with a proline, and the substituting at position 9 of the antigenic unit is a replacement with arginine; the substituting at position 1 of the antigenic unit is a replacement with an alanine, and the substituting at position 9 of the antigenic unit is a replacement with arginine; the substituting at position 1 of the antigenic unit is a replacement with a serine, and the substituting at position 9 of the antigenic unit is a replacement with threonine; the substituting at position 1 of the antigenic unit is a replacement with a glutamate, and the substituting at position 9 of the antigenic unit is a replacement with glutamate; the substituting at position 1 of the antigenic unit is a replacement with a proline, and the substituting at position 9 of the antigenic unit is a replacement with threonine the substituting at position 1 of the antigenic unit is a replacement with a glutamate, and the substituting at position 9 of the antigenic unit is a replacement with lysine or an arginine; or the substituting at position 1 of the antigenic unit is a replacement with a glutamate or a lysine, and the substituting at position 9 of the antigenic unit is a replacement with histidine; or any combination thereof when more than one repeating antigenic unit is mutated.

In a related aspect, disclosed herein are de-epitoped HMW glutenin proteins comprising at least two additional substitution mutations at any of positions 3, 4, or 7, of said repeating antigenic unit. In a further related aspect, the substitution at position 3 is a polar or positively charged amino acid; the substitution at position 4 is a negatively charged amino acid or glycine; the substitution at position 7 is a glycine; and any combination thereof when more than one repeating antigenic unit is mutated.

In a related aspect, disclosed herein are de-epitoped HMW glutenin proteins comprising at least two additional substitution mutations at any of positions 3, 5, or 8, of said antigenic unit. In a further related aspect, the substitution at position 3 is a polar or positively charged amino acid; the substitution at position 5 is a hydrophobic amino acid; the substitution at position 8 is to a small or aliphatic amino acid; and any combination thereof when more than one repeating antigenic unit is mutated. In another further related aspect, the substitution at position 5 is leucine and the substitution at position 3 is arginine.

In a related aspect, disclosed herein are de-epitoped HMW glutenin proteins wherein the number of mutated repeating antigenic units comprises at least 5-10 repeating antigenic units, and wherein the mutations within each repeating antigenic unit may be the same or different. In a further related aspect, the first mutation, or the second mutation, or both comprises a deletion. In certain aspects, the deletion comprises a deletion of all the amino acids at positions 1-9 of said repeating antigenic unit.

In a related aspect, disclosed herein are de-epitoped HMW glutenin proteins comprising the amino acid sequence set forth in any of SEQ ID NOs: 52, 102-107, 109-112, 114-116, 118-119, 126-134, 137-138, 140-142, 148-151, 155-168, or 170-172.

In another aspect, disclosed herein is an isolated polynucleotide encoding any of the de-epitoped HMW glutenin proteins described throughout. In another aspect, disclosed herein is an expression vector comprising the isolated polynucleotide encoding a de-epitoped HMW glutenin protein, operatively linked to a transcriptional regulatory sequence so as to allow expression of said de-epitoped high molecular weight glutenin in a cell.

In another aspect, disclosed herein is a method of producing de-epitoped HMW glutenin comprising culturing cells that comprise the expression vector of claim 18 under conditions allowing for expression of said de-epitoped HMW glutenin in said cells; expressing said de-epitoped HMW glutenin; and collecting said expressed de-epitoped HMW glutenin. In a related aspect, disclosed herein is a cell comprising any of the de-epitoped HMW glutenin proteins described herein.

In another aspect, disclosed herein is method of de-epitoping a HMW glutenin protein comprising mutating the amino acid residue at position 1 and 9 of at least one repeating antigenic unit of the glutenin, said repeating antigenic unit having an amino acid sequence selected from the group consisting of SEQ ID NOs: 1-33, 35, or 71-99, thereby generating a de-epitoped high molecular weight glutenin. In a related aspect, the mutating is carried out on at least five-ten of said repeating antigenic units. In a further related aspect, the mutating comprises substituting at least two amino acid residues or deleting amino acid residues. In another further related aspect, the mutating comprises substituting an amino acid at position 9 of said repeating antigenic unit, wherein said substituting comprises a replacement with a positively charged, a small amino acid, or a glutamate, or a combination thereof when more than one repeating antigenic unit is mutated. In certain aspects, the positively charged amino acid is arginine, histidine or lysine; and the small amino acid is serine or threonine. In a further aspect, when the mutating comprises substituting, said substituting at position 9 of said antigenic unit is a replacement with an arginine, histidine, lysine, threonine, or a glutamate, or a combination thereof when more than one repeating antigenic unit is mutated. In another further aspect, when said mutating comprises substituting, said substituting at position 1 of said antigenic unit comprises a replacement with a histidine, a proline, a serine, an alanine, a lysine, or a glutamate, or a combination thereof when more than one repeating antigenic unit is mutated. In related aspects, the substituting at position 1 of the antigenic unit is a replacement with histidine, and the substituting at position 9 of the antigenic unit is a replacement with histidine; the substituting at position 1 of the antigenic unit is a replacement with proline, and the substituting at position 9 of the antigenic unit is a replacement with arginine; the substituting at position 1 of the antigenic unit is a replacement with alanine, and the substituting at position 9 of the antigenic unit is a replacement with arginine; the substituting at position 1 of the antigenic unit is a replacement with serine, and the substituting at position 9 of the antigenic unit is a replacement with threonine; the substituting at position 1 of the antigenic unit is a replacement with glutamate, and the substituting at position 9 of the antigenic unit is a replacement with glutamate; the substituting at position 1 of the antigenic unit is a replacement with proline, and the substituting at position 9 of the antigenic unit is a replacement with threonine; the substituting at position 1 of the antigenic unit is a replacement with a glutamate, and the substituting at position 9 of the antigenic unit is a replacement with lysine or an arginine; or the substituting at position 1 of the antigenic unit is a replacement with a glutamate or a lysine, and the substituting at position 9 of the antigenic unit is a replacement with histidine; or any combination thereof when more than one repeating antigenic unit is mutated.

In related aspects, disclosed herein is a method of de-epitoping a HMW glutenin further comprising mutating at least two amino acids at positions 3, 4, or 7 of said repeating antigenic unit. In a related aspect, the mutating comprises substituting amino acid residues or deleting amino acid residues. In some aspects, when said mutating is substituting, said substituting at position 3 is replacing the amino acid with a polar or positive amino acid; or said substituting at position 4 is replacing the amino acid with a negatively charged amino acid or glycine; or said substituting at position 7 is replacing the amino acid with glycine; or any combination thereof when more than one repeating antigenic unit is mutated.

In related aspects, disclosed herein is a method of de-epitoping a HMW glutenin further comprising mutating at least two amino acids at positions 3, 5, or 8 of said repeating antigenic unit. In a related aspect, said mutating is substituting, said substituting at position 3 is replacing the amino acid with a polar or a positively charged amino acid; or said substituting at position 5 is replacing the amino acid with a hydrophobic amino acid; or. said substituting at position 8 is replacing the amino acid with a small or aliphatic amino acid; or any combination thereof when more than one repeating antigenic unit is mutated. In a further related aspect, said substituting at position 5 is replacing the amino acid with a leucine and said substituting at position 3 is replacing the amino acid with an arginine.

In related aspects, disclosed herein is a method of de-epitoping a HMW glutenin comprising mutations wherein said mutations comprise deletions, and said deleting comprises deleting the amino acids at positions 1-9 of the repeating antigenic unit.

In another related aspect, disclosed herein is method of de-epitoping a HMW glutenin protein, wherein the de-epitoped HMW glutenin comprises an amino acid sequence set forth in any of SEQ ID NOs: 52, 102-107, 109-112, 114-116, 118-119, 126-134, 137-138, 140-142, 148-151, 155-168, or 170-172.

In a related aspect, a method of de-epitoping a HMW glutenin further comprises analyzing the binding affinity of said de-epitoped high molecular weight glutenin for HLA-DQ2.5, HLA-DQ8, HLA-DQ2.2, HLA-DQ7.5; and identifying said de-epitoped high molecular weight glutenin as comprising reduced immunogenicity, when said de-epitoped HMW glutenin comprises a lower affinity for T-cells derived from a celiac patient compared with the binding affinity for T-cells derived from a celiac patient of a corresponding non-mutated high molecular weight glutenin; or when said de-epitoped high molecular weight glutenin activates T-cells derived from a celiac patient to a lesser extent than a corresponding non-mutated high molecular weight glutenin activates T cells derived from said celiac patient, as measured using a HLA-DQ-peptide tetramer-based assay or by an interferon-7 ELISA assay; or a combination thereof, or identifying said de-epitoped high molecular weight glutenin as not comprising reduced immunogenicity: when said modified antigenic units of de epitoped HMW glutenin bind to MHCII class DQ2, DQ2.2, DQ2.5, DQ7.5 or DQ8 with an IC50 of less than 30 μM.

In a related aspect, a method of de-epitoping a HMW glutenin further comprises analyzing the binding affinity of said de-epitoped high molecular weight glutenin for IgE binding; identifying those said de-epitoped high molecular weight glutenin having reduced IgE binding; assaying the identified de-epitoped high molecular weight glutenin having reduced IgE binding, for degranulation of basophiles, wherein a reduced response compared with the response of a wheat extract correlates with de-epitoped high molecular weight glutenin proteins comprising reduced allergenicity.

In one aspect, disclosed herein is a flour comprising any of the de-epitoped HMW glutenin described herein. In another aspect, disclosed herein is dough comprising a flour comprising any of the de-epitoped HMW glutenin proteins described herein.

In another aspect, disclosed herein is modified wheat expressing any of the de-epitoped HMW glutenin described herein.

In another aspect, disclosed herein is modified corn plant expressing any of the de-epitoped HMW glutenin described herein.

In another aspect, disclosed herein is flour derived from the wheat or the corn plant comprising a de-epitoped HMW glutenin.

### BRIEF DESCRIPTION OF THE DRAWINGS

The subject matter regarded as the de-epitoped high molecular weight (HMW) glutenin, methods of production thereof, and uses thereof is particularly pointed out and distinctly claimed in the concluding portion of the specification. The de-epitoped high molecular weight (HMW) glutenin, methods of production thereof, and uses thereof, however, both as to organization and method of operation, together with objects, features, and advantages thereof, may best be understood by reference to the following detailed description when read with the accompanying drawings in which:

FIGS. 1A-1I present the amino acid sequences of representative embodiments of wild-type non-de-epitoped HMW glutenin polypeptides as set forth in SEQ ID NOs: 43-48 and 152-154. Antigenic units of FIGS. 1A-1F are shown in bold lettering. The boxed antigenic unit is the literature described HMW glutenin epitope, set forth as SEQ ID NO: 1. The italicized amino acids at position 1 and 9, are known to sometimes undergo deamidation, when tissue transglutaminase converts them to glutamate in the small intestine. In certain embodiments, the mutations to a repeating antigenic unit includes mutations at positions 1 and 9, wherein the mutation may comprise a Glutamine (Q) to Glutamate (E) conversion. Underline indicates residues that differ from the literature described HMW glutenin epitope set forth in SEQ ID NO: 1.

FIG. 2 presents embodiments of 13-mer peptides that comprise mutations within a repeating antigenic unit. Bold indicates a mutant amino acid residue. Underlining indicates positions 1 and 9 of the antigenic unit (epitope core). Peptides identified as SEQ ID NOs: 37 and 39 bind to DQ7.5 (Data not shown).

FIG. 3 shows modifications to HMW-GS immunogenic peptide (modified peptides are SEQ ID NOs: 51 and 53) lead to abolishment of T-cell activation.

FIG. 4 shows modifications to HMW-GS immunogenic peptide (modified peptides are SEQ ID NOs: 52, 54, and 55) lead to abolishment of T-cell activation.

FIG. 5 shows modifications to HMW-GS immunogenic peptide (modified peptide is SEQ ID NO: 56) lead to abolishment of T-cell activation.

FIG. 6 shows modifications to HMW-GS immunogenic peptide (modified peptide is SEQ ID NO: 143) lead to abolishment of T-cell activation.

FIG. 7 shows modifications to HMW-GS immunogenic peptide (modified peptide is SEQ ID NO: 146) lead to abolishment of T-cell activation.

FIG. 8 shows modifications to HMW-GS immunogenic peptide (modified peptide is SEQ ID NO: 147) lead to abolishment of T-cell activation.

FIG. 9 shows modifications to HMW-GS immunogenic peptide (modified peptides are SEQ ID NOs: 174, 169, 145, 144, and 176) lead to abolishment of T-cell activation.

FIGS. 10A and 10B present the evaluation of specific IgE from wheat allergic patient sera to wheat extract and HMW Dy10 (SEQ ID NO:48 WT; SEQ ID NO: 103 and SEQ ID NO: 169 DE) and HMW Dx5 (SEQ ID NO: 47 WT; SEQ ID NOs: 128 and 102 DE) recombinant proteins: wild type [WT] and de-epitoped [DE]. Sera specific IgE binding was evaluated by a standard ELISA protocol. Recombinant HMW proteins or wheat extract were immobilized on a solid surface (microplate) and sixty-three clinically validated wheat allergy human patient sera were sampled in triplicates. Data is presented as a normalized binding signal: IgE signal of tested sera (O.D. 450) were divided by the IgE signal of the negative control sera (O.D. 450), on a logarithmic axis. The mean normalized binding for the recombinant HMW proteins and the wheat extract is marked by a black bar. For all groups, Dunnett's multiple comparisons test to the wheat extract group was performed. **** represent P≤0.0001, and NS stands for non-significant p-value.

### DETAILED DESCRIPTION

In the following detailed description, numerous specific details are set forth in order to provide a thorough understanding of the de-epitoped high molecular weight (HMW) glutenin polypeptides described herein, methods of producing these HMW glutenin polypeptides, methods of de-epitoping HMW glutenin, and uses of the de-epitoped HMW glutenin polypeptides. However, it will be understood by those skilled in the art that the methods of de-epitoping HMW glutenin, the de-epitoped HMW glutenins described, methods of producing de-epitoped HMW glutenins, and use thereof may be practiced without these specific details. In other instances, well-known methods, procedures, and components have not been described in detail so as not to obscure the description of the present disclosure.

Celiac disease (CD) is a long-term autoimmune disorder that primarily affects the small intestine. Classic symptoms include gastrointestinal problems such as chronic diarrhea, abdominal distention, malabsorption, loss of appetite and among children failure to grow normally. This often begins between six months and two years of age. Non-classic symptoms are more common, especially in people older than two years. There may be mild or absent gastrointestinal symptoms, a wide number of symptoms involving any part of the body or no obvious symptoms.

CD is caused by a reaction to gluten found in wheat and in other grains such as barley and rye. Gluten are proteins naturally found in some grains e.g., wheat, barley, and rye, and includes for example HMW glutenin. Upon exposure to gluten, an abnormal immune response may lead to the production of several different autoantibodies that can affect a number of different organs. In the small bowel, this causes an inflammatory reaction and may produce shortening of the villi lining the small intestine.

Diagnosis is typically made by a combination of blood antibody tests and intestinal biopsies, helped by specific genetic testing. While the disease is caused by a permanent intolerance to wheat proteins, it is not a form of wheat allergy.

The methods and uses of de-epitoped HMW glutenin as described herein also relate to other forms of gluten sensitivity. The term “CD”, in some embodiments, may encompass other forms of gluten sensitivity.

HMW glutenin proteins comprise repeating antigenic units comprising Celiac Disease (CD) reactive epitopes. Various ranges of repeating antigenic units are comprised in HMW glutenin proteins. In some embodiments, a HMW glutenin protein comprises 10-30 repeating antigenic subunits. In some embodiments, an HMW glutenin protein comprises more than 30 repeating antigenic subunits. In certain embodiments, a CD reactive epitope corresponds with or overlaps with an IgE allergenic epitope.

In some embodiments, de-epitoping HMW glutenin, comprises mutating antigenic units comprising CD relevant T-cell epitopes. In some embodiments, de-epitoping HMW glutenin provides HMW glutenin polypeptides having reduced allergenicity. In some embodiments, de-epitoping HMW glutenin provides HMW glutenin polypeptides having reduced immunogenicity. In some embodiments, de-epitoping HMW glutenin antigenic units comprising these CD relevant T-cell epitopes provides hypoallergenic HMW glutenin polypeptides. In some embodiments, these de-epitoped HMW glutenin polypeptides comprise a lower binding affinity to T-cells and show reduced activation of T-cells. Thus, these de-epitoped HMW glutenin polypeptides may be used to produce doughs and food products compatible with the diet of a human subject suffering from CD.

The name HMW-GS appears in the literature as the full name of the HMW-glutenin family of proteins, dividing glutenins into higher (HMW) and lower molecular weight (LMW) subunits, i.e. when you extract all glutenins from wheat, they can be divided into these two families, these two “subunits”.

As used herein the term “HMW glutenin protein”, “HMW glutenin polypeptide”, “HMW-GS”, and “HMW glutenin”, may in some embodiments be used interchangeably having all the same meanings and qualities. 20 [0050] A skilled artisan would appreciate that a “high molecular weight glutenin” (“HMW glutenin”) may encompass a grass gluten protein comprising a repeating motif (for example but not limited to PGQGQQ (SEQ ID NO: 100), GYYPTS[P/L]QQ (SEQ ID NO: 101 and SEQ ID NO: 180) and/or GQQ) and an extensive central elastomeric domain flanked by two terminal non-repetitive domains that form disulphide cross-links. The repeating motifs typically appear more than 10, more than 15, or even more than 20 times in the protein. Exemplary grasses from which the HMW glutenin protein may be derived include, but are not limited to wheat (e.g. einkorn wheat), spelt and barley. In some embodiments, the HMW glutenin is a wheat high molecular weight glutenin.

A skilled artisan would appreciate that the term “antigenic unit” encompasses the repeating antigenic units present in HMW glutenin comprising CD relevant T-cell epitopes. In some embodiments, the term “antigenic unit” encompasses a single antigenic unit comprising a CD relevant T-cell epitope. In some embodiments, the term “antigenic unit” encompasses multiple antigenic units each comprising a CD relevant T-cell epitope. In some embodiments, an “antigenic unit” comprises a HMW glutenin antigenic peptide subunit. In some embodiments, the term “antigenic unit” is used interchangeably with the terms “repeating antigenic unit”, and the like. In certain embodiments, an HMW glutenin comprises repeating antigenic units, wherein the amino acid sequence of the repeating units may have between 50%-100% identity. In some embodiments, the amino acid sequence of repeating antigenic units comprised within a HMW glutenin do not have 100% amino acid sequence identity. In some embodiments, a repeating antigenic unit comprises a repeating motif (for example but not limited to PGQGQQ (SEQ ID NO: 100), GYYPTS[P/L]QQ (SEQ ID NO: 101 and SEQ ID NO: 180) and/or GQQ).

A skilled artisan would appreciate that the term “epitope” may be used interchangeably with the term “antigenic determinant”, “CD relevant T-cell epitope”, and “CD epitope” and the like, having all the same meanings and qualities, and may encompass a site on an antigen to which a T-cell specifically binds.

T-cell epitopes are formed by contiguous amino acids in a protein or peptide. Epitopes formed from contiguous amino acids (linear epitopes) are typically retained on exposure to denaturing solvents. An epitope typically includes at least 3, 4, 5, 6, 7, 8, 9, 10, 11, 12, 13, 14 or 15 amino acids in a unique spatial conformation. In some embodiments, the epitope is as small as possible while still maintaining immunogenicity. Immunogenicity is indicated by the ability to elicit an immune response, as described herein, for example, by the ability to bind an MHC class II molecule and to induce a T cell response, e.g., as measured by T cell cytokine production. In some embodiments, an antigenic unit comprises an epitope core.

The molecules that transport and present peptides on the cell surface are referred to as proteins of the major histocompatibility complex (MHC). MHC proteins are classified into two types, referred to as MHC class I and MHC class II. The structures of the proteins of the two MHC classes are very similar; however, they have very different functions. Proteins of MHC class I are present on the surface of almost all cells of the body, including most tumor cells. MHC class I proteins are loaded with antigens that usually originate from endogenous proteins or from pathogens present inside cells, and are then presented to naive or cytotoxic T-lymphocytes (CTLs). MHC class II proteins are present on dendritic cells, B-lymphocytes, macrophages and other antigen-presenting cells. They mainly present peptides, which are processed from external antigen sources, i.e. outside of the cells, to T-helper (Th) cells. T-Cell receptors are capable of recognizing and binding peptides complexed with the molecules of MHC class I or II. Each T-lymphocyte expresses a specific T-cell receptor which is capable of binding specific MHC/peptide complexes. In some embodiments, a de-epitoped HMW glutenin or antigenic subunit thereof, as disclosed herein, demonstrates reduced binding to a MHC II protein.

Antigen presenting cells (APC) are cells which present peptide fragments of protein antigens in association with MHC molecules on their cell surface. Some APCs may activate antigen specific T cells. Examples of APCs include, but are not limited to dendritic cells, B-cells and macrophages.

According to some embodiments, an antigenic unit disclosed herein comprises a T-cell epitope. In some embodiments, an antigenic unit comprises the minimal required T-cell epitope. The minimal peptide length required but not always sufficient for binding MHC II is nine, as this is the number of amino-acids that interact directly with the MHC II binding cleft. A lack of binding to MHC II will result in the absence of a peptide-MHC II complex to be recognized by T-cell receptors, making this implicitly also the minimal length for T-cell activation. In some embodiments, the T-cell epitope is a celiac disease-associated epitope (CD-associated epitope)—i.e. the epitope is presented on antigen presenting cells (APCs) of a celiac patient. As used throughout, the terms “CD relevant T-cell epitope” and “CD-associated epitope”, and the like may be used interchangeably having all the same meanings and qualities.

Within a protein sequence, for example the sequences of a HMW glutenin polypeptide, there are continuous CD-relevant epitopes, which are linear sequences of amino acids that may be bound by an antigen presenting cell (APC).

A skilled artisan would appreciate that the terms “de-epitoped high molecular weight glutenin”, “de-epitoped HMW glutenin”, “modified HMW glutenin”, “modified de-epitoped high molecular weight glutenin”, and “modified de-epitoped HMW glutenin” and the like, may be used interchangeably having all the same qualities and meaning, and that de-epitoped HMW glutenin may in certain embodiments, encompass a modified HMW glutenin that has reduced or abolished binding with an APC, for example T-cells (as compared to a T-cell binding to its wild-type counterpart) due to mutation(s) at one or more epitopes recognized by T-cells.

In some embodiments, a de-epitoped HMW glutenin described herein, has reduced immunogenicity as compared to its wild-type counterpart. For example, celiac disease (CD) is mediated by T-cell epitopes that are modified in de-epitoped HMW glutenin in order to avoid HLA binding and presentation to T-cells.

In some embodiments, a digested de-epitoped HMW glutenin described herein has reduced or abolished binding with MHC class II molecules. In some embodiments, a de-epitoped HMW glutenin described herein has reduced immunogenicity as compared to its wild-type counterpart. In some embodiments, a de-epitoped HMW glutenin described herein is hypoallergenic as compared to its wild-type counterpart.

As used herein, an “allergen” may encompass a substance, protein or non-protein, capable of inducing allergy or specific hypersensitivity. In some embodiments, an allergen comprises a HMW glutenin polypeptide.

As used herein, “allergenicity” or “allergenic” encompasses the ability of an antigen or allergen to induce an abnormal immune response, which is an overreaction and different from a normal immune response in that it does not result in a protective/prophylaxis effect but instead causes physiological function disorder or tissue damage. In some embodiments, a HMW glutenin allergen induces an abnormal immune response causing diseases and or tissue damage, for example but not limited to wheat dependent exercise induced anaphylaxis (WDEIA).

As used herein, an “immunogen” may encompass a molecule that is capable of eliciting an immune response.

As used herein, “immunogenicity” may encompass the ability or the extent to which a substance is able to stimulate an immune response.

Thus, a skilled artisan would recognize that “antigenicity” is the ability to specifically combine with the final products of the immune response, for example, secreted antibodies and/or surface receptors on T cells, and “immunogenicity” is the ability to induce a humoral and/or cell-mediated immune response. Significantly, although all molecules that are immunogenic are also antigenic, the reverse is not true.

As used herein, “hypoallergenic” refers to a substance having little or reduced likelihood of causing an allergic response. In some embodiments, a de-epitoped HMW glutenin is hypoallergenic comprising a reduced or minimal allergic response.

In some embodiments, the present disclosure provides de-epitoped HMW glutenin that were mutated to diminish or abolish one or more CD relevant T-cell epitopes. In some embodiment, the mutation does not affect the biophysical and/or functional characteristics of the de-epitoped HMW glutenin, for example but not limited to de-epitoped HMW glutenin's ability to contribute to the elasticity of dough. The mutations in some embodiments comprise substitution or deletion mutations. A deletion, for example, may comprise the removal of a single amino acid that is crucial for an allergic and or a CD related response, or of a whole mapped epitope region.

Six general classes of amino acid side chains have been categorized and include: Class I (Cys); Class II (Ser, Thr, Ala, Gly); Class III (Asn, Asp, Gln, Glu); Class IV (His, Arg, Lys); Class V (Ile, Leu, Val, Met); and Class VI (Phe, Tyr, Trp). In addition, a Pro may be substituted in the variant structures. Conservative amino acid substitution refers to substitution of an amino acid in one class by an amino acid of the same class. For example, substitution of an Asp for another class III residue such as Asn, Gln, or Glu, is a conservative substitution. Non-conservative amino acid substitution refers to substitution of an amino acid in one class with an amino acid from another class; for example, substitution of an Ala, a class II residue, with a class III residue such as Asp, Asn, Glu, or Gln. Methods of substitution mutations at the nucleotide or amino acid sequence level are well-known in the art.

The term “modifying,” or “modification,” as used herein, refers to changing two or more amino acids in an antigenic unit. In some embodiments, 2, 3, 4, 5, 6, 7, 8, 9, or more amino acids are modified within an antigenic unit. The change can be produced by substituting or deleting an amino acid at one or more positions. The change can be produced using known techniques, such as PCR mutagenesis. In some embodiments, the modification of repeating antigenic units comprises identical substitutions or deletions of each antigenic unit. In other embodiments, the modification of repeating antigenic units comprises different substitutions or deletions of each antigenic unit.

### De-Epitoped High Molecular Weight (HMW) Glutenin

In some embodiments, disclosed herein is a de-epitoped high molecular weight (HMW) glutenin comprising a first mutation at position 1 and a second mutation at position 9 of a repeating antigenic unit. In some embodiments, disclosed herein is a de-epitoped high molecular weight (HMW) glutenin comprising a first mutation at position 1 and a second mutation at position 9 of a repeating antigenic unit, wherein the amino acid sequence of said repeating non-mutated antigenic unit is set forth in any of SEQ ID NOs: 1-33, 35, or 71-99.

In some embodiments, the grasses from which the HMW glutenin protein may be derived include, but are not limited to wheat (e.g., einkorn wheat), spelt, and barley. In one embodiment, the HMW glutenin is a wheat HMW glutenin. In one embodiment, the HMW glutenin is a spelt HMW glutenin. In one embodiment, the HMW glutenin is a barley HMW glutenin.

One skilled in the art would appreciate that HMW glutenins comprise repeating antigenic units, wherein de-epitoping may comprise mutations to individual antigenic units or may comprise mutations to multiple antigenic units of the HMW glutenin. In some embodiments, the mutations found in an antigenic unit are the same as the mutations present in another antigenic unit of the HMW glutenin. In some embodiments, the mutations found in an antigenic unit are different from the mutations present in another antigenic unit of the HMW glutenin. In some embodiments, the mutations found in an antigenic unit are different from the mutations present in at least one other antigenic unit of the HMW glutenin and are the same as the mutations present in at least one other antigenic unit of the HMW glutenin. In some embodiments, a de-epitoped HMW glutenin comprises antigenic units comprising different mutations. In some embodiments, a de-epitoped HMW glutenin comprises antigenic units comprising the same mutations. In some embodiments, a de-epitoped HMW glutenin comprises a mix of antigenic units comprising the same and different mutations.

In some embodiments, a single HMW glutenin comprises multiple repeating antigenic units, wherein the sequences of the antigenic units may be the identical. In some embodiments, a single HMW glutenin comprises multiple repeating antigenic units wherein the sequences of the antigenic units may be the different. In some embodiments, a single HMW glutenin comprises multiple repeating antigenic units wherein the sequences of the antigenic units include antigenic units with identical sequences and antigenic units with different sequences.

In some embodiments, repeating antigenic units comprise antigenic units that repeat between about 5-30 times. In some embodiments, repeating antigenic units comprise antigenic units that repeat between about 5-20 times. In some embodiments, repeating antigenic units comprise antigenic units that repeat between about 5-10 times. In some embodiments, repeating antigenic units comprise antigenic units that repeat between about 5-15 times. In some embodiments, repeating antigenic units comprise antigenic units that repeat between about 10-15 times. In some embodiments, repeating antigenic units comprise antigenic units that repeat between about 10-20 times. In some embodiments, repeating antigenic units comprise antigenic units that repeat between about 15-20 times. In some embodiments, repeating antigenic units comprise antigenic units that repeat between about 10-30 times. In some embodiments, repeating antigenic units comprise antigenic units that repeat between about 15-30 times. In some embodiments, repeating antigenic units comprise antigenic units that repeat between about 20-30 times. In some embodiments, repeating antigenic units comprise antigenic units that repeat more than 30 times.

In some embodiments, repeating antigenic units comprise antigenic units that repeat more than about 5-30 times. In some embodiments, repeating antigenic units comprise antigenic units that repeat more than about 5-20 times. In some embodiments, repeating antigenic units comprise antigenic units that repeat more than about 5-10 times. In some embodiments, repeating antigenic units comprise antigenic units that repeat more than about 5-15 times. In some embodiments, repeating antigenic units comprise antigenic units that repeat more than about 10-15 times. In some embodiments, repeating antigenic units comprise antigenic units that repeat more than about 10-20 times. In some embodiments, repeating antigenic units comprise antigenic units that repeat more than about 15-20 times. In some embodiments, repeating antigenic units comprise antigenic units that repeat between more than 10-30 times. In some embodiments, repeating antigenic units comprise antigenic units that repeat between more than 15-30 times. In some embodiments, repeating antigenic units comprise antigenic units that repeat between more than 20-30 times.

In some embodiments, a HMW glutenin comprises a grass gluten protein comprising a repeating motif for example but not limited to the sequences set forth as PGQGQQ (SEQ ID NO: 100), GYYPTS[P/L]QQ (SEQ ID NO: 101 and SEQ ID NO: 180), and/or GQQ) and an extensive central elastomeric domain flanked by two terminal non-repetitive domains that form disulphide cross-links. In some embodiments, a repeating antigenic unit of an HMW glutenin comprises a repeating motif selected from SEQ ID NO: 100, SEQ ID NO: 101, or GQQ.

In some embodiments, a repeating antigenic unit comprises a tripeptide wherein the amino acid sequence is GQQ. In some embodiments, a repeating antigenic unit comprises a hexapeptide, wherein the amino acid sequence is set forth in SEQ ID NO: 100. In some embodiments, a repeating antigenic unit comprises a nonapeptide, wherein the amino acid sequence is set forth in SEQ ID NO: 101. In some embodiments, an antigenic unit comprises a known amino acid sequence of an antigenic unit. In some embodiments, an antigenic unit comprises an amino acid sequence similar to that of a known sequence of an antigenic unit. In some embodiments, an antigenic unit comprises an amino acid sequence having 70-99.9% identity with that of a known sequence of a HMW glutenin repeating antigenic unit, determined using BlastP software of the National Center of Biotechnology Information (NCBI) using default parameters. In some embodiments, an antigenic unit comprises an amino acid sequence having 70%, 75%, 80%, 85%, 90%, 95%, 97%, or 99%, identity with that of a known sequence of a HMW glutenin repeating antigenic unit

In some embodiments, a repeating antigenic unit comprises a nonapeptide, wherein the amino acid sequence is selected from the sequences set forth in SEQ ID NOs: 1-33, 35, 71-75 and 77-99. In some embodiments, a repeating antigenic unit comprises a dodecapeptide, wherein the amino acid sequence is set forth in SEQ ID NOs: 76. In some embodiments, a repeating antigenic unit comprises an amino acid sequence selected from the sequences set forth in SEQ ID NOs: 1-33, 35, and 71-99.

In some embodiments, an antigenic unit comprises a CD-relevant epitope comprising a determinant that is recognized by lymphocytes. The CD-relevant epitope can be a peptide which is presented by a major histocompatibility complex (MHC) molecule and is capable of specifically binding to a T-cell receptor. In certain embodiments, a CD epitope is a region of a T cell immunogen that is specifically bound by a T-cell receptor. In certain embodiments, a CD epitope may include chemically active surface groupings of molecules such as amino acids, sugar side chains, phosphoryl, or sulfonyl groups. In certain embodiments, a CD epitope may have specific three-dimensional structural characteristics and/or specific charge characteristics.

The T cell CD-relevant epitope comprised within a repeating antigenic unit, may in some embodiments comprise a short peptide that is bound to a class II MHC molecule, thus forming a ternary complex that can be recognized by a T-cell bearing a matching T-cell receptor binding to the MHC/peptide complex with appropriate affinity. T-cell epitopes that bind to MHC class II molecules are typically about 12-30 amino acids in length, but can be longer. In the case of peptides that bind to MHC class II molecules, the same peptide and corresponding T cell epitope may share a common core segment, but differ in the overall length due to flanking sequences of differing lengths upstream of the amino-terminus of the epitope core sequence and downstream of its carboxy terminus, respectively. The T-cell epitope may be classified as an immunogen if it elicits an immune response. In certain embodiments, the T cell CD-relevant epitope of HMW glutenin is present in a repeating antigenic unit. In certain embodiments, the T cell CD-relevant epitope of HMW glutenin is present in a repeating antigenic unit comprising the amino acid sequence as set forth in any of SEQ ID NOs: 1-33, 35, or 71-99.

As used herein, the term “T cell receptor” or “TCR” refers to a complex of membrane proteins that participate in the activation of T cells in response to the presentation of antigen. The TCR is responsible for recognizing antigens bound to major histocompatibility complex molecules. TCR is composed of a heterodimer of an alpha (a) and beta (p) chain, although in some cells the TCR consists of gamma and delta chains. TCRs may exist in alpha/beta and gamma/delta forms, which are structurally similar but have distinct anatomical locations and functions. Each chain is composed of two extracellular domains, a variable and constant domain. TCRs in the present invention may exist in a variety of forms including different fragments of TCR.

The term “T cell immunogen” refers to an agent (for example a protein or a portion thereof) that is capable of eliciting a T cell mediated immune response. In some embodiments, a CD relevant T cell immunogen comprises at least one T cell CD epitope. In some embodiments, a T cell immunogen comprises a wheat protein or portions thereof, such as a gluten protein. In some embodiments, the T cell immunogen comprises HMW glutenin from wheat.

In certain embodiments, wherein a CD relevant T cell epitope is the same as or overlaps with an IgE binding wheat allergy epitope, an antigenic unit comprises a determinant that is recognized by IgE antibodies derived from serum obtained from a human subject suffering from a wheat allergy.

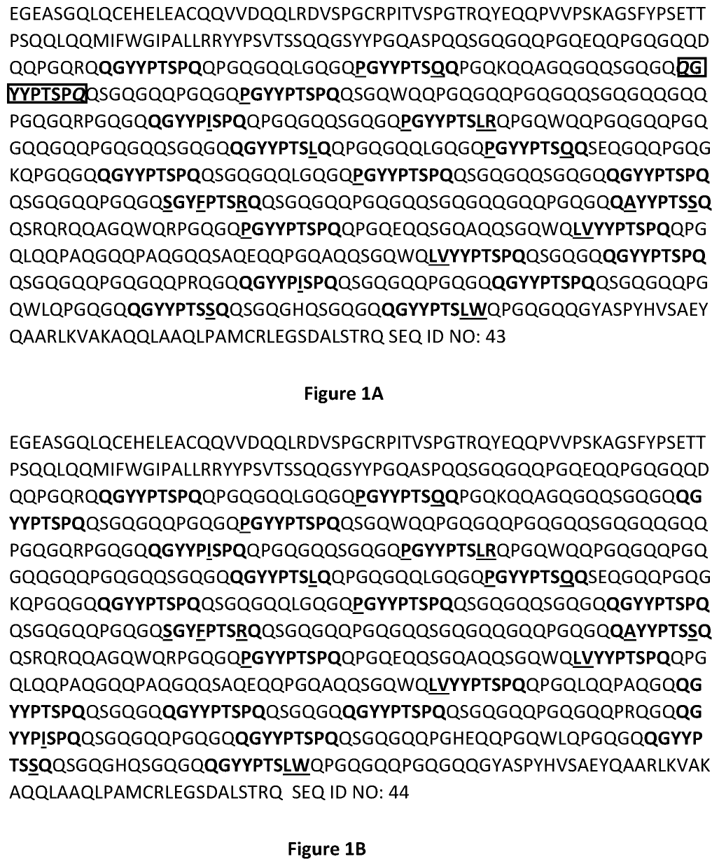

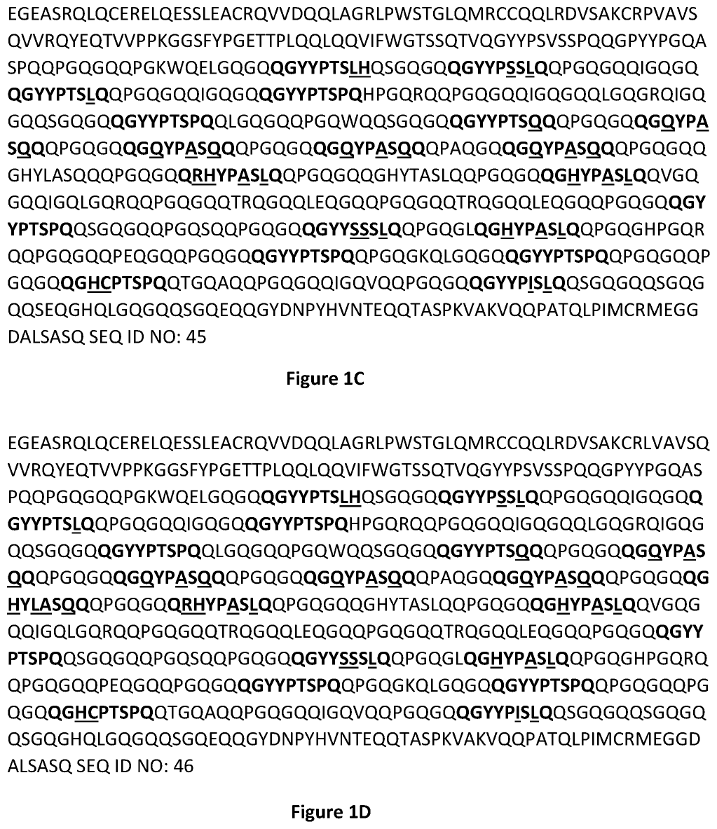

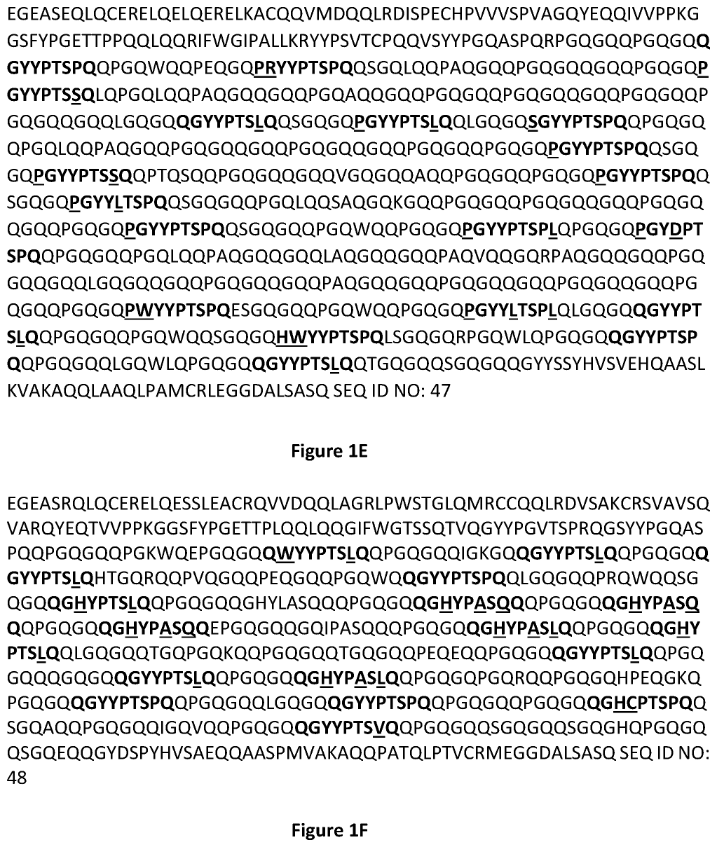

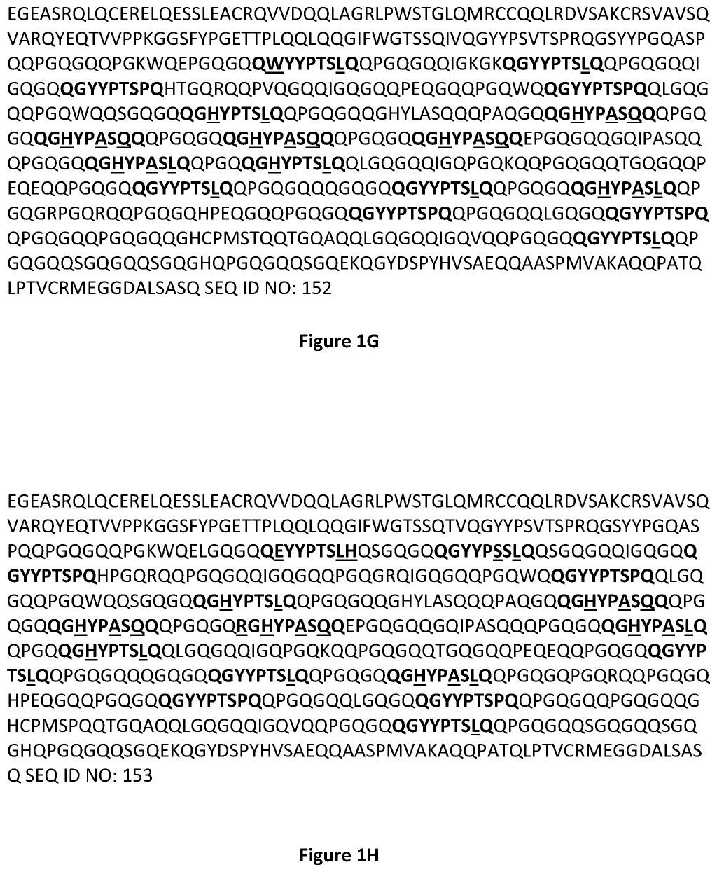

A skilled artisan would appreciate that an antibody (Ab) epitope can be formed both from contiguous amino acids or noncontiguous amino acids juxtaposed by tertiary folding of a protein. Epitopes formed from contiguous amino acids (linear epitopes) are typically retained on exposure to denaturing solvents, whereas epitopes formed by tertiary folding (conformational epitopes) are typically lost upon treatment with denaturing solvents. An epitope typically includes at least 3, 4, 5, 6, 7, S, 9, 10, 11, 12, 13, 14 or 15 amino acids in a unique spatial conformation. Allergenicity to an epitope is indicated by the capacity to induce allergen specific IgE antibodies in susceptible individuals (subject suffering from wheat allergies). In some embodiments, an antigenic unit comprises an epitope core. Within a protein sequence, for example the sequences of a HMW glutenin polypeptide, there are continuous epitopes, which are linear sequences of amino acids that may be bound by an antibody, or discontinuous epitopes that similarly may be bound by an antibody, but which exist only when the protein is folded into a particular conformation. In certain embodiments, the repeating antigenic unit comprise those set forth in any of the sequences SEQ ID NOs: 1-33 or 35. In certain embodiments, the repeating antigenic unit comprise those set forth in any of the sequences SEQ ID NOs: 71-99. In certain embodiments, the repeating antigenic unit comprise those set forth in any of the sequences SEQ ID NOs: 1-33, 35, or 71-99. In certain embodiments, the repeating antigenic unit comprise the amino acid sequence set forth in SEQ ID NO: 1. (FIG. 1A, boxed antigenic unit)

In some embodiments, a non-de-epitoped HMW glutenin comprises a wild-type (WT) HMW glutenin. In some embodiments, the amino acid sequence of a non-de-epitoped HMW glutenin is selected from the sequences set forth in SEQ ID NOs: 43-48 and 152-154. (FIGS. 1A-1I). In some embodiments, the amino acid sequence of a non-de-epitoped HMW glutenin is selected from the sequences having at least 50% identity with the amino acid sequences set forth in SEQ ID NOs: 43-48 and 152-154. In some embodiments, the amino acid sequence of a non-de-epitoped HMW glutenin is selected from the sequences having at least 50%-99% identity with the amino acid sequences set forth in SEQ ID NOs: 43-48 and 152-154. In some embodiments, the amino acid sequence of a non-de-epitoped HMW glutenin is selected from the sequences having at least 60%, 65%, 70%, 75%, 80%, 85%, 90% or 95%, or even 99% identity with any of the amino acid sequences set forth in SEQ ID NOs: 43-48 and 152-154. In some embodiments, the amino acid sequence of a non-de-epitoped HMW glutenin is selected from the sequences set forth in SEQ ID NOs: 43-48 and 152-154, or is selected from the amino acid sequences having at least 50% identity with the sequences set forth in any of SEQ ID NOs: 43, 44, 45, 46, 47, 48, 152, 153, and 154.

The “percent identity” of two amino acid sequences may be determined using the algorithm of Karlin and Altschul, Proc. Natl. Acad. Sci. USA 87:2264-68, 1990, modified as in Karlin and Altschul, Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, word length=3 to obtain amino acid sequences homologous to the protein molecules of interest. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used. BLAST nucleotide searches can be performed with the NBLAST nucleotide program parameters set, e.g., for score=100, wordlength=12 to obtain nucleotide sequences homologous to a nucleic acid molecule described herein. BLAST protein searches can be performed with the XBLAST program parameters set, e.g., to score 50, wordlength=3 to obtain amino acid sequences homologous to a protein molecule described herein. To obtain gapped alignments for comparison purposes, Gapped BLAST can be utilized as described in Altschul S F et al., (1997) Nuc Acids Res 25: 3389 3402. Alternatively, PSI BLAST or PHI BLAST can be used to perform an iterated search which detects distant relationships between molecules (Id.). When utilizing BLAST, Gapped BLAST, PSI Blast and PHI Blast programs, the default parameters of the respective programs (e.g., of XBLAST and NBLAST) can be used (see, e.g., National Center for Biotechnology Information (NCBI) on the worldwide web, ncbi(dot)nlm(dot)nih(dot)gov). Another specific, non-limiting example of a mathematical algorithm utilized for the comparison of sequences is the algorithm of Myers and Miller, 1988, CABIOS 4:11 17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps, such that any software for protein sequence alignment can be used. In calculating percent identity, typically only exact matches are counted.

In some embodiments, the non-de-epitoped HMW glutenin polypeptide comprises an amino acid sequence that is at least 50%, at least 55%, at least 60%, at least 65%, at least 70%, at least 75%, or at least 80%, at least 85%, at least 90%, or at least 95%, or at least 99% identical to a polypeptide set forth in any of SEQ ID NOs: 43-48 and 152-154, as determined using BlastP software of the National Center of Biotechnology Information (NCBI) using default parameters.

In certain embodiments, the modified de-epitoped HMW glutenins disclosed herein have a sequence that is at least 50%, 55%, 60%, 65%, 70%, 75%, 80%, 85%, 90%, 91%, 92%, 93%, 94%, 95%, 96%, 97%, 98%, 99% identical to any one of the sequences as set forth in SEQ ID NOs: 43-48 and 152-154.

One skilled in the art would appreciate that the term “modified” as used herein to describe a HMW glutenin encompasses a de-epitoped HMW glutenin as disclosed herein. In some embodiments, the terms “modified HMW glutenin”, “modified de-epitoped glutenin”, and “de-epitoped glutenin” and the like, may all be used interchangeable having the same meanings and qualities.

In some embodiments, a de-epitoped HMW glutenin as disclosed herein, comprises a protein comprising at least 2 mutations within at least one repeating antigenic unit that has been identified to comprise a CD relevant T-cell epitope, wherein a de-epitoped HMW glutenin protein binds with less affinity to its relevant MHC protein than a wild-type counterpart, and/or wherein the de-epitoped HMW glutenin protein activates T-cells to a lesser extent than its wild-type counterpart, as further described herein below. In some embodiments, a de-epitoped HMW glutenin as disclosed herein, comprises a protein comprising at least 2 amino acid deletion mutations within at least one repeating antigenic unit that has been identified to comprise a CD relevant T-cell epitope, wherein a de-epitoped HMW glutenin protein binds with less affinity to its relevant MHC protein than a wild-type counterpart, and/or wherein the de-epitoped HMW glutenin protein activates T-cells to a lesser extent than its wild-type counterpart, as further described herein below. In some embodiments, a de-epitoped HMW glutenin as disclosed herein, comprises a protein comprising at least 2 substitution mutations within at least one repeating antigenic unit that has been identified to comprise a CD relevant T-cell epitope, wherein a de-epitoped HMW glutenin protein binds with less affinity to its relevant MHC protein than a wild-type counterpart, and/or wherein the de-epitoped HMW glutenin protein activates T-cells to a lesser extent than its wild-type counterpart, as further described herein below.

In certain embodiment, the protein comprises a mutation in at least two repeating antigenic units, at least 3, 4, 5, 6, 7, 8, 9, 10, 11, 12, 13, 14, 15, 16, 17, 18, 19, 20, 21, 22, 23, 24, 25, 26, 27, 28, 29, 30 antigenic units. In some embodiment, the protein comprises a mutation in more than two repeating antigenic units, more than 3, 4, 5, 6, 7, 8, 9, 10, 11, 12, 13, 14, 15, 16, 17, 18, 19, 20, 21, 22, 23, 24, 25, 26, 27, 28, 29, or 30 antigenic units. In some embodiment, the protein comprises a mutation in two repeating antigenic units, 3, 4, 5, 6, 7, 8, 9, 10, 11, 12, 13, 14, 15, 16, 17, 18, 19, 20, 21, 22, 23, 24, 25, 26, 27, 28, 29, or 30 antigenic units. In some embodiment, the protein comprises a mutation in all repeating antigenic units. In some embodiment, the protein comprises a deletion of all repeating antigenic units.

In some embodiment, the protein comprises a mutation in 1-30 antigenic units. In some embodiment, the protein comprises a mutation in 1-20 antigenic units. In some embodiment, the protein comprises a mutation in 1-5 antigenic units. In some embodiment, the protein comprises a mutation in 1-10 antigenic units. In some embodiment, the protein comprises a mutation in 1-15 antigenic units. In some embodiment, the protein comprises a mutation in 10-15 antigenic units. In some embodiment, the protein comprises a mutation in 5-20 antigenic units. In some embodiment, the protein comprises a mutation in 5-10 antigenic units. In some embodiment, the protein comprises a mutation in 5-15 antigenic units. In some embodiment, the protein comprises a mutation in 10-20 antigenic units. In some embodiment, the protein comprises a mutation in 15-20 antigenic units. In some embodiment, the protein comprises a mutation in more than 20 antigenic units. In some embodiment, the protein comprises a mutation in more than 5-30 times. In some embodiment, the protein comprises a mutation in more than 10-30 times. In some embodiment, the protein comprises a mutation in more than 15-30 times. In some embodiment, the protein comprises a mutation in more than 20-30 times. In some embodiment, the protein comprises a mutation in more than 5-30 times.

In some embodiments, at least 10%, at least 20%, at least 30%, at least 40%, at least 50%, at least 60%, at least 70%, at least 80%, at least 90%, or all of the antigenic units present in the de-epitoped HMW glutenin protein are mutated (i.e. de-epitoped). In some embodiments, 10%, 20% 30%, 40%, 50%, 60%, 70%, 80%, 90%, or all of the antigenic units present in the de-epitoped HMW glutenin protein are mutated.

In some embodiments, the de-epitoped HMW glutenin proteins described herein comprise at least one essential physical property that is also present in its wild-type counterpart. Thus, for example in the case of HMW glutenin, de-epitoped high molecular weight glutenin has a beneficial property to contribute to the elasticity of bread dough. Other examples of beneficial properties include dough rising promoting ability of HMW glutenin, and dough strengthening ability of HMW glutenin.

In some embodiments, a de-epitoped HMW glutenin comprises mutations within a repeating antigenic unit. In some embodiments, a de-epitoped HMW glutenin comprises a mutation of one or more amino acid residues of a wheat HMW glutenin polypeptide, where said mutation is within one or more identified CD-relevant epitope or within one or more proposed CD-relevant epitope.

In some embodiments, a HMW glutenin comprises 2, 3, 4, 5, 6, 7, 8, 9, 10, 11, 12, 13, 14, 15, 16, 17, 18, 19, 20, 21, 22, 23, 24, or 25 or more mutations of amino acid residues with the repeating antigenic units of the HMW glutenin polypeptide. In some embodiments, the one or more mutations destroy one or more (or all) of the identified T cell CD-relevant epitopes on the polypeptide (i.e. the mutations cause the T cell CD-relevant epitopes to bind with less affinity to its relevant MHC protein than its wild-type counterpart and/or activates T cells to a lesser extent than its wild-type counterpart).

In some embodiments, a de-epitoped HMW glutenin comprises a first mutation at position 1 and a second mutation at position 9 of a repeating antigenic unit. In some embodiments, a de-epitoped HMW glutenin comprises a first mutation and a second mutation as any of positions 1-9 of a repeating antigenic unit.

Methods for making polypeptides comprising one or more mutations are well known to one of ordinary skill in the art. In some embodiments, the one or more mutations are conservative mutations. In some embodiments, the one or more mutations are non-conservative mutations. In some embodiments, the one or more mutations are a mixture of conservative and non-conservative mutations.

In some embodiments, the one or more mutations comprise a substitution, a deletion, or an insertion, or a combination thereof. In some embodiments, a mutation within an antigenic unit comprises a substitution. In some embodiments, a mutation within an antigenic unit comprises a deletion. In some embodiments, a mutation within an antigenic unit comprises an insertion. One skilled in the art would appreciate that one antigenic unit may comprise certain mutations, while another antigenic unit may comprise different mutations.

In some embodiments, a deletion mutation comprises deleting at least one amino acid of the repeating antigenic unit, two amino acids of the repeating antigenic unit, three amino acids of the repeating antigenic unit, four amino acids of the repeating antigenic unit, five amino acids of the repeating antigenic unit, six amino acids of the repeating antigenic unit, seven amino acids of the repeating antigenic unit, eight amino acids of the repeating antigenic unit, or all the amino acids of the repeating unit. In some embodiments, a deletion mutation comprises deleting a repeat antigenic unit.

In some embodiments, at least 20% of the repeating antigenic units are fully deleted, at least 30% of the repeating antigenic units are fully deleted, at least 40% of the repeating antigenic units are fully deleted, at least 50% of the repeating antigenic units are fully deleted, at least 60% of the repeating antigenic units are fully deleted, at least 70% of the repeating antigenic units are fully deleted, at least 80% of the repeating antigenic units are fully deleted, or at least 90% of the repeating antigenic units are fully deleted. In some embodiments, all of the repeating antigenic units are fully deleted.

In some embodiments, an antigenic unit of a de-epitoped HMW glutenin comprises a first mutation at position 1 and a second mutation at position 9, wherein the first mutation, or the second mutation, or both comprise a deletion mutation.

In some embodiments, a HMW glutenin comprises a deletion mutation of 1, 2, 3, 4, 5, 6, 7, 8, 9, 10, 11, 12, 13, 14, 15, or all of the amino acid residues within a repeating antigenic unit of the HMW glutenin polypeptide. In some embodiments, deletion destroys one or more (or all) of the identified T cell CD-relevant epitopes on the polypeptide (i.e. the mutations cause the T cell CD-relevant epitopes to bind with less affinity to its relevant MHC protein than its wild-type counterpart and/or activates T cells to a lesser extent than its wild-type counterpart).

In some embodiments, a de-epitoped HMW glutenin comprises the amino acid sequence set forth in any of SEQ ID NOs: 102-107, 167-168, or SEQ ID NOs: 170-172. In some embodiments, all of the repeating antigenic units of a de-epitoped HMW glutenin as set forth in any of SEQ ID NOs: 102-107, 167-168, or 170-172, are fully deleted. In some embodiments, the amino acid sequence of the de-epitoped high molecular weight glutenin is 90% identical, 91% identical, 92% identical, 93% identical, 94% identical, 95% identical, 96% identical, 97% identical, 98% identical, 99% identical, to any one of the sequences as set forth in SEQ ID NOs: 102-107, 167-168, or 170-172, wherein all the repeating units are fully deleted, and wherein identity is determined using BlastP software of the National Center of Biotechnology Information (NCBI) using default parameters or like software programs.

In some embodiments, an antigenic unit of a de-epitoped HMW glutenin comprises a first mutation at position 1 and a second mutation at position 9, wherein the first mutation, or the second mutation, or both comprise a substitution mutation.

In some embodiments, the ninth amino acid of at least one of the antigenic units is replaced with a positively charged or small amino acid. In some embodiments, the ninth amino acid of at least two of the antigenic units is replaced with a positively charged or small amino acid. In some embodiments, the ninth amino acid of at least three of the antigenic units is replaced with a positively charged or small amino acid. In some embodiments, the ninth amino acid of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 antigenic units is replaced with a positively charged or small amino acid. In some embodiments, the ninth amino acid of 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 antigenic units is replaced with a positively charged or small amino acid. In some embodiments, the ninth amino acid of more than 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 antigenic units is replaced with a positively charged or small amino acid.

In some embodiments, the ninth amino acid of at least 50%, 60%, 70%, 80%, 90%, or all of the antigenic units is replaced with a positively charged or small amino acid.

As used herein, the term “positively charged amino acids” encompasses histidine, lysine and arginine. In some embodiments, the positively charged amino acid is an arginine. In some embodiments, the positively charged amino acid is a histidine. In some embodiments, the positively charged amino acid is an arginine or a histidine. In some embodiments, the positively charged amino acid is a lysine.

As used herein, the term “small amino acids” encompasses serine, threonine, alanine, glycine, and valine. In some embodiments, the small amino acid is a serine. In some embodiments, the small amino acid is a threonine. In some embodiments, the small amino acid is an alanine. In some embodiments, the small amino acid is a glycine. In some embodiments, the small amino acid is a valine.

In some embodiments, the ninth amino acid of at least one of the antigenic units is replaced with a threonine, a serine, an arginine, a histidine, or a glutamate. In some embodiments, the ninth amino acid of at least two of the antigenic units is replaced with a threonine, a serine, an arginine, a histidine, or a glutamate. In some embodiments, the ninth amino acid of at least three of the antigenic units is replaced with a threonine, a serine, an arginine, a histidine, or a glutamate. In some embodiments, the ninth amino acid of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 antigenic units is replaced with a threonine, a serine, an arginine, a histidine, or a glutamate. In some embodiments, the ninth amino acid of 1, 2, 3, 4, 5, 6, 7, 8, 9, 10, 11, 12, 13, 14, 15, 16, 17, 18, 20, 21, 22, 23, 24, 25, 26, 27, 28, 29, or 30 antigenic units is replaced with a threonine, a serine, an arginine, a histidine, or a glutamate. In some embodiments, the ninth amino acid of more than 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 antigenic units is replaced with a threonine, a serine, an arginine, a histidine, or a glutamate.

In some embodiments, the ninth amino acid of at least one of the antigenic units is replaced with a threonine or a glutamate. In some embodiments, the ninth amino acid of at least two of the antigenic units is replaced with a threonine or a glutamate. In some embodiments, the ninth amino acid of at least three of the antigenic units is replaced with a threonine or a glutamate. In some embodiments, the ninth amino acid of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 antigenic units is replaced with a threonine or a glutamate. In some embodiments, the ninth amino acid of 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 antigenic units is replaced with a threonine or a glutamate. In some embodiments, the ninth amino acid of more than 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 antigenic units is replaced with a threonine or a glutamate.

In some embodiments, the ninth amino acid of at least 50%, 60%, 70%, 80%, 90%, or all of the antigenic units is replaced with a threonine or a glutamate. In some embodiments, the ninth amino acid of at least 50%, 60%, 70%, 80%, 90%, or all of the antigenic units is replaced with a threonine, a serine, an arginine, a histidine, or a glutamate.

In some embodiments, the ninth amino acid of at least one of the antigenic units is replaced with an arginine, a histidine, a threonine or a glutamate. In some embodiments, the ninth amino acid of at least two of the antigenic units is replaced with an arginine, a histidine, a threonine or a glutamate. In some embodiments, the ninth amino acid of at least three of the antigenic units is replaced with an arginine, a histidine, a threonine or a glutamate. In some embodiments, the ninth amino acid of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 antigenic units is replaced with an arginine, a histidine, a threonine or a glutamate. In some embodiments, the ninth amino acid of 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 antigenic units is replaced with an arginine, a histidine, a threonine or a glutamate. In some embodiments, the ninth amino acid of more than 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 antigenic units is replaced with an arginine, a histidine, a threonine or a glutamate.

In some embodiments, the ninth amino acid of at least 50%, 60%, 70%, 80%, 90%, or all of the antigenic units is replaced with an arginine, a histidine, a threonine or a glutamate.

In some embodiments, the ninth amino acid of at least one of the antigenic units is replaced with a histidine or an arginine. In some embodiments, the ninth amino acid of at least two of the antigenic units is replaced with a histidine or an arginine. In some embodiments, the ninth amino acid of at least three of the antigenic units is replaced with a histidine or an arginine. In some embodiments, the ninth amino acid of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 antigenic units is replaced with a histidine or an arginine. In some embodiments, the ninth amino acid of 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 antigenic units is replaced with a histidine or an arginine. In some embodiments, the ninth amino acid of more than 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 antigenic units is replaced with a histidine or an arginine.

In some embodiments, the ninth amino acid of at least 50%, 60%, 70%, 80%, 90%, or all of the antigenic units is replaced with a histidine or an arginine.

In some embodiments, the ninth amino acid of more than one of the antigenic units is replaced with an arginine, a histidine, a threonine or a glutamate. In some embodiments, the ninth amino acid of more than two of the antigenic units is replaced with an arginine, a histidine, a threonine or a glutamate. In some embodiments, the ninth amino acid of more than three of the antigenic units is replaced with an arginine, a histidine, a threonine or a glutamate. In some embodiments, the ninth amino acid of more than 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 antigenic units is replaced with an arginine, a histidine, a threonine or a glutamate. In some embodiments, the ninth amino acid of 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 antigenic units is replaced with an arginine, a histidine, a threonine or a glutamate.

In some embodiments, the ninth amino acid of more than 50%, 60%, 70%, 80%, 90%, or all of the antigenic units is replaced with an arginine, a histidine, a threonine or a glutamate.

In some embodiments, the ninth amino acid of at least one of the antigenic units is replaced with a serine. In some embodiments, the ninth amino acid of at least two of the antigenic units is replaced with a serine. In some embodiments, the ninth amino acid of at least three of the antigenic units is replaced with a serine. In some embodiments, the ninth amino acid of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 antigenic units is replaced with a serine. In some embodiments, the ninth amino acid of 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 antigenic units is replaced with a serine. In some embodiments, the ninth amino acid of more than 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 antigenic units is replaced with a serine.

In some embodiments, the ninth amino acid of at least 50%, 60%, 70%, 80%, 90%, or all of the antigenic units is replaced with a serine.

In some embodiments of a de-epitoped HMW glutenin disclosed herein, the second mutation (position 9) comprises a substation mutation replacing the amino acid residue at position 9 of said repeating antigenic unit with a positively charged amino acid or a small amino acid; or a glutamate; or a combination of thereof when more than one repeating antigenic unit is mutated.

In some embodiments of a de-epitoped HMW glutenin disclosed herein, the second mutation (position 9) comprises a substitution mutation comprising replacement with an arginine or histidine; or a serine or threonine; or a combination thereof when more than one repeating antigenic unit is mutated.

In some embodiments of a de-epitoped HMW glutenin disclosed herein, the first mutation (position 1) comprises a substitution mutation wherein the first amino acid of at least one of the repeating antigenic units is replaced with a histidine, a proline, a serine, an alanine, a lysine, or a glutamate. In some embodiments, the first amino acid of at least one of the repeating antigenic units is replaced with a histidine. In some embodiments, the first amino acid of at least one of the repeating antigenic units is replaced with a proline. In some embodiments, the first amino acid of at least one of the repeating antigenic units is replaced with a serine. In some embodiments, the first amino acid of at least one of the repeating antigenic units is replaced with an alanine. In some embodiments, the first amino acid of at least one of the repeating antigenic units is replaced with a lysine. In some embodiments, the first amino acid of at least one of the repeating antigenic units is replaced with a glutamate.

In some embodiments of a de-epitoped HMW glutenin disclosed herein, the first mutation comprises a substitution mutation wherein the first amino acid of at least two of the repeating antigenic units is replaced with a histidine, a proline, a serine, an alanine, a lysine, or a glutamate. In some embodiments, the first amino acid of at least two of the repeating antigenic units is replaced with a histidine. In some embodiments, the first amino acid of at least two of the repeating antigenic units is replaced with a proline. In some embodiments, the first amino acid of at least two of the repeating antigenic units is replaced with a serine. In some embodiments, the first amino acid of at least two of the repeating antigenic units is replaced with an alanine. In some embodiments, the first amino acid of at least two of the repeating antigenic units is replaced with a lysine. In some embodiments, the first amino acid of at least two of the repeating antigenic units is replaced with a glutamate.

In some embodiments of a de-epitoped HMW glutenin disclosed herein, the first mutation comprises a substitution mutation wherein the first amino acid of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 of the repeating antigenic units is replaced with a histidine, a proline, a serine, an alanine, a lysine, or a glutamate. In some embodiments, the first amino acid of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 of the repeating antigenic units is replaced with a histidine. In some embodiments, the first amino acid of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 of the repeating antigenic units is replaced with a proline. In some embodiments, the first amino acid of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 of the repeating antigenic units is replaced with a serine. In some embodiments, the first amino acid of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 of the repeating antigenic units is replaced with an alanine. In some embodiments, the first amino acid of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 of the repeating antigenic units is replaced with a lysine. In some embodiments, the first amino acid of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 of the repeating antigenic units is replaced with a glutamate.

In some embodiments of substitution mutations of a repeating antigenic unit comprised in a de-epitoped HMW glutenin, replacements comprise
(i) substituting at position 1 of the antigenic unit with histidine, and substituting at position 9 of the antigenic unit with histidine;
(ii) substituting at position 1 of the antigenic unit with proline, and substituting at position 9 of the antigenic unit with arginine;
(iii) substituting at position 1 of the antigenic unit with alanine, and substituting at position 9 of the antigenic unit with arginine;
(iv) substituting at position 1 of the antigenic unit with serine, and substituting at position 9 of the antigenic unit with threonine;
(v) substituting at position 1 of the antigenic unit with glutamate, and substituting at position 9 of the antigenic unit with glutamate;
(vi) substituting at position 1 of the antigenic unit with proline, and substituting at position 9 of the antigenic unit with threonine;
(vii) said substituting at position 1 of said antigenic unit is a replacement with a glutamate, and said substituting at position 9 of said antigenic unit is a replacement with lysine or an arginine; or
(viii) said substituting at position 1 of said antigenic unit is a replacement with a glutamate or a lysine, and said substituting at position 9 of said antigenic unit is a replacement with histidine; or
(ix) any combination of (i)-(viii) when more than one repeating antigenic unit is mutated.

In some embodiments, a de-epitoped HMW glutenin disclosed herein, comprises a mutation at position 1 of a repeating antigenic unit, the mutation comprising a substitution mutation that replaces the amino acid residue at position 1 of the repeating antigenic unit with a histidine, a proline, a serine, an alanine, a lysine, or a glutamate.

In some embodiments, a de-epitoped HMW glutenin disclosed herein, comprises mutations a position 1 and position 9 of a repeating antigenic unit, wherein
said substituting at position 1 of said antigenic unit is a replacement with a histidine, and said substituting at position 9 of said antigenic unit is a replacement with histidine;
said substituting at position 1 of said antigenic unit is a replacement with a proline, and said substituting at position 9 of said antigenic unit is a replacement with arginine;
said substituting at position 1 of said antigenic unit is a replacement with an alanine, and said substituting at position 9 of said antigenic unit is a replacement with arginine;
said substituting at position 1 of said antigenic unit is a replacement with a serine, and said substituting at position 9 of said antigenic unit is a replacement with threonine;
said substituting at position 1 of said antigenic unit is a replacement with a glutamate, and said substituting at position 9 of said antigenic unit is a replacement with glutamate; or
said substituting at position 1 of said antigenic unit is a replacement with a proline, and said substituting at position 9 of said antigenic unit is a replacement with threonine;
said substituting at position 1 of said antigenic unit is a replacement with a glutamate, and said substituting at position 9 of said antigenic unit is a replacement with lysine or an arginine; or
   said substituting at position 1 of said antigenic unit is a replacement with a glutamate or a lysine, and said substituting at position 9 of said antigenic unit is a replacement with histidine; or
   any combination of (i)-(viii) when more than one repeating antigenic unit is mutated.

In some embodiments, a de-epitoped HMW glutenin disclosed herein, comprises mutations in position 1 and position 9 of a repeating antigenic unit, wherein the substituting at position 1 of said antigenic unit is a replacement with a histidine, and said substituting at position 9 of said antigenic unit is a replacement with histidine. In some embodiments, a de-epitoped HMW glutenin disclosed herein, comprises mutations a position 1 and position 9 of a repeating antigenic unit, wherein the substituting at position 1 of said antigenic unit is a replacement with a proline, and said substituting at position 9 of said antigenic unit is a replacement with arginine. In some embodiments, a de-epitoped HMW glutenin disclosed herein, comprises mutations a position 1 and position 9 of a repeating antigenic unit, wherein the substituting at position 1 of said antigenic unit is a replacement with an alanine, and said substituting at position 9 of said antigenic unit is a replacement with arginine. In some embodiments, a de-epitoped HMW glutenin disclosed herein, comprises mutations a position 1 and position 9 of a repeating antigenic unit, wherein the substituting at position 1 of said antigenic unit is a replacement with a serine, and said substituting at position 9 of said antigenic unit is a replacement with threonine. In some embodiments, a de-epitoped HMW glutenin disclosed herein, comprises mutations a position 1 and position 9 of a repeating antigenic unit, wherein the substituting at position 1 of said antigenic unit is a replacement with a glutamate, and said substituting at position 9 of said antigenic unit is a replacement with glutamate. In some embodiments, a de-epitoped HMW glutenin disclosed herein, comprises mutations a position 1 and position 9 of a repeating antigenic unit, wherein the substituting at position 1 of said antigenic unit is a replacement with a proline, and said substituting at position 9 of said antigenic unit is a replacement with threonine. In some embodiments, a de-epitoped HMW glutenin disclosed herein, comprises mutations in position 1 and position 9 of a repeating antigenic unit, wherein the substituting at position 1 of said antigenic unit is a replacement with a glutamate, and said substituting at position 9 of said antigenic unit is a replacement with lysine or an arginine. In some embodiments, a de-epitoped HMW glutenin disclosed herein, comprises mutations a position 1 and position 9 of a repeating antigenic unit, wherein the substituting at position 1 of said antigenic unit is a replacement with a glutamate or a lysine, and said substituting at position 9 of said antigenic unit is a replacement with histidine.

In some embodiments, an antigenic unit comprises a first mutation at position 1 and a second mutation at position 9, wherein the first mutation, or the second mutation, or both comprise an insertion mutation.

In some embodiments a de-epitoped HMW glutenin disclosed herein having a mutation at positions 1 and 9, further comprises additional mutations within a repeating antigenic unit, wherein the positions at which further substitutions may be made comprise positions, 3, 4, and 7. In some embodiments a de-epitoped HMW glutenin having a mutation at positions 1 and 9, further comprises at least two additional mutations within a repeating antigenic unit, wherein the positions at which the at least two substitutions may be made comprise positions, 3, 4, and 7. In some embodiments a de-epitoped HMW glutenin having a mutation at positions 1 and 9, further comprises three additional mutations within a repeating antigenic unit, wherein the positions at which the three substitutions may be made comprise positions, 3, 4, and 7.

In some embodiments, the third amino acid of at least one of the antigenic units may be replaced with a positively charged or polar amino acid. In some embodiments, the third amino acid of at least 2, 3, 4, 5, 6, 7, 8 9, 10, 11, 12, 13, 14, 15, 16, 17, 18, 19, 20, 21, 22, 23, 24, 25, 26, 27, 28, 29, or 30 of the antigenic units may be replaced with a positively charged or polar amino acid. In some embodiments, the third amino acid of at least 50%, 60%, 70%, 80%, 90% or all of the antigenic units may be replaced with a positively charged or polar amino acid.

In some embodiments, the third amino acid of at least one of the antigenic units may be replaced with a positively charged amino acid. In some embodiments, the third amino acid of at least 2, 3, 4, 5, 6, 7, 8 9, 10, 11, 12, 13, 14, 15, 16, 17, 18, 19, 20, 21, 22, 23, 24, 25, 26, 27, 28, 29, or 30 of the antigenic units may be replaced with a positively charged amino acid. In some embodiments, the third amino acid of at least 50%, 60%, 70%, 80%, 90% or all of the antigenic units may be replaced with a positively charged amino acid.

In some embodiments, the third amino acid of at least one of the antigenic units may be replaced with a polar amino acid. In some embodiments, the third amino acid of at least 2, 3, 4, 5, 6, 7, 8 9, 10, 11, 12, 13, 14, 15, 16, 17, 18, 19, 20, 21, 22, 23, 24, 25, 26, 27, 28, 29, or 30 of the antigenic units may be replaced with a polar amino acid. In some embodiments, the third amino acid of at least 50%, 60%, 70%, 80%, 90% or all of the antigenic units may be replaced with a polar amino acid.

In some embodiments of a de-epitoped HMW glutenin, an amino acid at a fourth position of at least one of the antigenic units may be replaced with a negatively charged amino acid or a glycine. In some embodiments of a de-epitoped HMW glutenin, an amino acid at a fourth position of at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 of the antigenic units may be replaced with a negatively charged amino acid or a glycine. In some embodiments of a de-epitoped HMW glutenin, an amino acid at a fourth position of at least 50%, 60%, 70%, 80%, 90% or all of the antigenic units may be replaced with a negatively charged amino acid or a glycine.

In some embodiments of a de-epitoped HMW glutenin, an amino acid at a fourth position of at least one of the antigenic units may be replaced with a negatively charged amino acid. In some embodiments of a de-epitoped HMW glutenin, an amino acid at a fourth position of at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 of the antigenic units may be replaced with a negatively charged amino acid. In some embodiments of a de-epitoped HMW glutenin, an amino acid at a fourth position of at least 50%, 60%, 70%, 80%, 90% or all of the antigenic units may be replaced with a negatively charged amino acid.

In some embodiments of a de-epitoped HMW glutenin, an amino acid at a fourth position of at least one of the antigenic units may be replaced with a glycine. In some embodiments of a de-epitoped HMW glutenin, an amino acid at a fourth position of at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 of the antigenic units may be replaced with a glycine. In some embodiments of a de-epitoped HMW glutenin, an amino acid at a fourth position of at least 50%, 60%, 70%, 80%, 90% or all of the antigenic units may be replaced with a glycine.

In some embodiments of a de-epitoped HMW glutenin, the seventh amino acid of at least one of the antigenic units may be replaced with glycine. In some embodiments of a de-epitoped HMW glutenin, the seventh amino acid of at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 of the antigenic units may be replaced with glycine. In some embodiments of a de-epitoped HMW glutenin, the seventh amino acid of at least 50%, 60 00 70%, 80%, 90% or all of the antigenic units is replaced with a glycine.

In some embodiments of a de-epitoped HMW glutenin, the seventh amino acid of more than one of the antigenic units may be replaced with glycine. In some embodiments of a de-epitoped HMW glutenin, the seventh amino acid of more than 2, 3, 4, 5, 6, 7, 8, 9, 10, 11, 12, 13, 14, 15, 16, 17, 18, 19, 20, 21, 22, 23, 24, 25, 26, 27, 28, 29, or 30 of the antigenic units may be replaced with glycine. In some embodiments of a de-epitoped HMW glutenin, the seventh amino acid of more than 50%, 60%, 70%, 80%, 90% or all of the antigenic units is replaced with a glycine.

In some embodiments of a de-epitoped HMW glutenin, the seventh amino acid of one of the antigenic units may be replaced with glycine. In some embodiments of a de-epitoped HMW glutenin, the seventh amino acid of 2, 3, 4, 5, 6, 7, 8, 9, 10, 11, 12, 13, 14, 15, 16, 17, 18, 19, 20, 21, 22, 23, 24, 25, 26, 27, 28, 29, or 30 of the antigenic units may be replaced with glycine. In some embodiments of a de-epitoped HMW glutenin, the seventh amino acid of 50%, 60%, 70%, 80 00 90% or all of the antigenic units is replaced with a glycine.

As used herein, the term “polar amino acids” may encompass amino acids serine, threonine, tyrosine, asparagine, and glutamine. In some embodiments, a polar amino acid comprises a serine. In some embodiments, a polar amino acid comprises a threonine. In some embodiments, a polar amino acid comprises an asparagine. In some embodiments, a polar amino acid comprises a tyrosine.

As used herein, the term “negatively charged” encompasses, aspartic acid and glutamic acid. In some embodiments, the negatively charged amino acid is an aspartic acid. In some embodiments, the negatively charged amino acid is a glutamic acid.

In some embodiments, a de-epitoped HMW glutenin disclosed herein, comprises at least two additional substitution mutations at any of positions 3, 4, and 7 of the repeating antigenic unit. In some embodiments, a de-epitoped HMW glutenin disclosed herein, comprises three additional substitution mutations at positions 3, 4, and 7 of the repeating antigenic unit.

In some embodiments, a de-epitoped HMW glutenin disclosed herein comprises at least two additional substitution mutations at any of positions 3, 4, and 7, comprises a substitution at position 3 that is a polar or positively charged amino acid; or a substitution at position 4 that is a negatively charged amino acid or glycine; or a substitution at position 7 that is a glycine; or any combination of thereof. In some embodiments, a de-epitoped HMW glutenin disclosed herein comprises three additional substitution mutations at positions 3, 4, and 7, comprising a substitution at position 3 that is a polar or positively charged amino acid; a substitution at position 4 that is a negatively charged amino acid or glycine; and a substitution at position 7 that is a glycine.

In some embodiments a de-epitoped HMW glutenin disclosed herein comprises additional mutations within a repeating antigenic unit, wherein the positions at which further substitutions may be made comprise positions 2, 3, and 8.

In some embodiments, the second amino acid of at least one of the antigenic units may be replaced with an aromatic amino acid or a positively charged amino acid. In some embodiments, the second amino acid of at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 of the antigenic units may be replaced with an aromatic amino acid or a positively charged amino acid. In some embodiments, the second amino acid of at least 50%, 60 00 70%, 80%, 90% or all of the antigenic units may be replaced as described above.

In some embodiments, the second amino acid of more than one of the antigenic units may be replaced with an aromatic amino acid or a positively charged amino acid. In some embodiments, the second amino acid of more than 2, 3, 4, 5, 6, 7, 8, 9, 10, 11, 12, 13, 14, 15, 16, 17, 18, 19, 20, 21, 22, 23, 24, 25, 26, 27, 28, 29, or 30 of the antigenic units may be replaced with an aromatic amino acid or a positively charged amino acid. In some embodiments, the second amino acid of more than 50%, 60%, 70%, 80%, 90% or all of the antigenic units may be replaced as described above.

In some embodiments, the second amino acid of one of the antigenic units may be replaced with an aromatic amino acid or a positively charged amino acid. In some embodiments, the second amino acid of 2, 3, 4, 5, 6, 7, 8, 9, 10, 11, 12, 13, 14, 15, 16, 17, 18, 19, 20, 21, 22, 23, 24, 25, 26, 27, 28, 29, or 30 of the antigenic units may be replaced with an aromatic amino acid or a positively charged amino acid. In some embodiments, the second amino acid of 50%, 60%, 70%, 80%, 90% or all of the antigenic units may be replaced as described above.

In some embodiments, the third amino acid of at least one of the antigenic units may be replaced with a polar amino acid or a positively charged amino acid. In some embodiments, the third amino acid of at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 of the antigenic units may be replaced with a polar amino acid or a positively charged amino acid. In some embodiments, the third amino acid of at least 50%, 60%, 70%, 80 00 90% or all of the antigenic units may be replaced as described above.

In some embodiments, the third amino acid of more than one of the antigenic units may be replaced with a polar amino acid or a positively charged amino acid. In some embodiments, the third amino acid of more than 2, 3, 4, 5, 6, 7, 8, 9, 10, 11, 12, 13, 14, 15, 16, 17, 18, 19, 20, 21, 22, 23, 24, 25, 26, 27, 28, 29, or 30 of the antigenic units may be replaced with a polar amino acid or a positively charged amino acid. In some embodiments, the third amino acid of more than 50%, 60%, 70%, 80%, 90% or all of the antigenic units may be replaced as described above.

In some embodiments, the third amino acid of one of the antigenic units may be replaced with a polar amino acid or a positively charged amino acid. In some embodiments, the third amino acid of 2, 3, 4, 5, 6, 7, 8, 9, 10, 11, 12, 13, 14, 15, 16, 17, 18, 19, 20, 21, 22, 23, 24, 25, 26, 27, 28, 29, or 30 of the antigenic units may be replaced with a polar amino acid or a positively charged amino acid. In some embodiments, the third amino acid of 50%, 60%, 70%, 80%, 90% or all of the antigenic units may be replaced as described above.

In some embodiments, the eighth amino acid of at least one of the antigenic units may be replaced with a small or aliphatic amino acid. In some embodiments, the eighth amino acid of at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 of the antigenic units may be replaced with a small or aliphatic amino acid. In some embodiments, the eighth amino acid of at least 50%, 60%, 70%, 80%, 90% or all of the antigenic units may be replaced as described above.

In some embodiments, the eighth amino acid of more than one of the antigenic units may be replaced with a small or aliphatic amino acid. In some embodiments, the eighth amino acid of more than 2, 3, 4, 5, 6, 7, 8, 9, 10, 11, 12, 13, 14, 15, 16, 17, 18, 19, 20, 21, 22, 23, 24, 25, 26, 27, 28, 29, or 30 of the antigenic units may be replaced with a small or aliphatic amino acid. In some embodiments, the eighth amino acid of more than 50%, 60%, 70%, 80%, 90% or all of the antigenic units may be replaced as described herein.

In some embodiments, the eighth amino acid of one of the antigenic units may be replaced with a small or aliphatic amino acid. In some embodiments, the eighth amino acid of 2, 3, 4, 5, 6, 7, 8, 9, 10, 11, 12, 13, 14, 15, 16, 17, 18, 19, 20, 21, 22, 23, 24, 25, 26, 27, 28, 29, or 30 of the antigenic units may be replaced with a small or aliphatic amino acid. In some embodiments, the eighth amino acid of 50%, 60%, 70%, 80%, 90% or all of the antigenic units may be replaced as described above.

In some embodiments, the amino acid at position 2 is replaced with a tryptophan and the amino acid at position 3 is replaced with a histidine.

As used herein, the term “aliphatic amino acid” may encompass amino acids methionine, valine, leucine, isoleucine and alanine. In some embodiments, an aliphatic amino acid comprises a methionine. In some embodiments, an aliphatic amino acid comprises a valine. In some embodiments, an aliphatic amino acid comprises a leucine. In some embodiments, an aliphatic amino acid comprises an isoleucine. In some embodiments, an aliphatic amino acid comprises an alanine.

As used herein, the term “aromatic amino acid” may encompass amino acids phenylalanine, tryptophan and tyrosine. In some embodiments, an aromatic amino acid comprises a phenylalanine. In some embodiments, an aromatic amino acid comprises a tryptophan. In some embodiments, an aromatic amino acid comprises a tyrosine.

In some embodiments, a de-epitoped HMW glutenin as disclosed herein having a mutation at positions 1 and 9, further comprises at least two additional substitution mutations at any of positions 2, 3, and 8 of the repeating antigenic unit. In some embodiments, wherein a de-epitoped HMW glutenin having a mutation at positions 1 and 9, further comprises at least two additional substitution mutations at any of positions 2, 3, and 8, the substitution at position 2 is an aromatic amino acid or positively charged amino acid; or the substitution at position 3 is a polar or positively charged amino acid; or the substitution at position 8 is to a small or aliphatic amino acid; or any combination thereof. In some embodiments, wherein a de-epitoped HMW glutenin comprises at least two additional substitution mutations at any of positions 2, 3, and 8, the substitution at position 2 is tryptophan and the substitution at position 3 is histidine.

In some embodiments, a de-epitoped HMW glutenin as disclosed herein having a mutation at positions 1 and 9, comprises three additional substitution mutations at positions 2, 3, and 8, of the repeating antigenic unit. In some embodiments, a de-epitoped HMW glutenin having a mutation at positions 1 and 9, comprises three additional substitution mutations at positions 2, 3, and 8, wherein the substitution at position 2 is an aromatic amino acid or positively charged amino acid; or wherein the substitution at position 3 is a polar or positively charged amino acid; or wherein the substitution at position 8 is to a small or aliphatic amino acid; or any combination thereof. In some embodiments, a de-epitoped HMW glutenin comprising a mutation at positions 1 and 9, comprises at least three additional substitution mutations at positions 2, 3, and 8, wherein the substitution at position 2 is tryptophan and the substitution at position 3 is histidine.

In some embodiments a de-epitoped HMW glutenin disclosed herein comprises additional mutations within a repeating antigenic unit, wherein the positions at which further substitutions may be made comprise positions 3, 5, and 8.

In some embodiments, the third amino acid of at least one of the antigenic units may be replaced with a polar or a positively charged amino acid. In some embodiments, the third amino acid of at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 of the antigenic units may be replaced with polar amino acid or a positively charged amino acid. In some embodiments, the third amino acid of at least 50%, 60%, 70%, 80%, 90% or all of the antigenic units may be replaced as described above.

In some embodiments, the third amino acid of more than one of the antigenic units may be replaced with polar amino acid or a positively charged amino acid. In some embodiments, the third amino acid of more than 2, 3, 4, 5, 6, 7, 8, 9, 10, 11, 12, 13, 14, 15, 16, 17, 18, 19, 20, 21, 22, 23, 24, 25, 26, 27, 28, 29, or 30 of the antigenic units may be replaced with polar amino acid or a positively charged amino acid. In some embodiments, the third amino acid of more than 50%, 60%, 70%, 80%, 90% or all of the antigenic units may be replaced as described above.

In some embodiments, the third amino acid of one of the antigenic units may be replaced with polar amino acid or a positively charged amino acid. In some embodiments, the third amino acid of 2, 3, 4, 5, 6, 7, 8, 9, 10, 11, 12, 13, 14, 15, 16, 17, 18, 19, 20, 21, 22, 23, 24, 25, 26, 27, 28, 29, or 30 of the antigenic units may be replaced with polar amino acid or a positively charged amino acid. In some embodiments, the third amino acid of 50%, 60%, 70%, 80%, 90% or all of the antigenic units may be replaced as described above.

In some embodiments, the fifth amino acid of at least one of the antigenic units may be replaced with a hydrophobic amino acid. In some embodiments, the fifth amino acid of at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 of the antigenic units may be replaced with a hydrophobic amino acid. In some embodiments, the fifth amino acid of at least 50%, 60%, 70%, 80%, 90% or all of the antigenic units may be replaced as described above.

In some embodiments, the fifth amino acid of more than one of the antigenic units may be replaced with a hydrophobic amino acid. In some embodiments, the fifth amino acid of more than 2, 3, 4, 5, 6, 7, 8, 9, 10, 11, 12, 13, 14, 15, 16, 17, 18, 19, 20, 21, 22, 23, 24, 25, 26, 27, 28, 29, or 30 of the antigenic units may be replaced with a hydrophobic amino acid. In some embodiments, the fifth amino acid of more than 50%, 60%, 70%, 80%, 90% or all of the antigenic units may be replaced as described above.

In some embodiments, the fifth amino acid of one of the antigenic units may be replaced with a hydrophobic amino acid. In some embodiments, the fifth amino acid of 2, 3, 4, 5, 6, 7, 8, 9, 10, 11, 12, 13, 14, 15, 16, 17, 18, 19, 20, 21, 22, 23, 24, 25, 26, 27, 28, 29, or 30 of the antigenic units may be replaced with a hydrophobic amino acid. In some embodiments, the fifth amino acid of 50%, 60%, 70%, 80%, 90% or all of the antigenic units may be replaced as described above.

In some embodiments, the eighth amino acid of at least one of the antigenic units may be replaced with a small or aliphatic amino acid. In some embodiments, the eighth amino acid of at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 of the antigenic units may be replaced with a small or aliphatic amino acid. In some embodiments, the eighth amino acid of at least 50%, 60%, 70%, 80%, 90% or all of the antigenic units may be replaced as described above.

In some embodiments, the eighth amino acid of more than one of the antigenic units may be replaced with a small or aliphatic amino acid. In some embodiments, the eighth amino acid of more than 2, 3, 4, 5, 6, 7, 8, 9, 10, 11, 12, 13, 14, 15, 16, 17, 18, 19, 20, 21, 22, 23, 24, 25, 26, 27, 28, 29, or 30 of the antigenic units may be replaced with a small or aliphatic amino acid. In some embodiments, the eighth amino acid of more than 50%, 60%, 70%, 80%, 90% or all of the antigenic units may be replaced as described herein.

In some embodiments, the eighth amino acid of one of the antigenic units may be replaced with a small or aliphatic amino acid. In some embodiments, the eighth amino acid of 2, 3, 4, 5, 6, 7, 8, 9, 10, 11, 12, 13, 14, 15, 16, 17, 18, 19, 20, 21, 22, 23, 24, 25, 26, 27, 28, 29, or 30 of the antigenic units may be replaced with a small or aliphatic amino acid. In some embodiments, the eighth amino acid of 50%, 60%, 70%, 80%, 90% or all of the antigenic units may be replaced as described above.

In some embodiments, a de-epitoped HMW glutenin as disclosed herein having a mutation at positions 1 and 9, further comprises at least two additional substitution mutations at any of positions 3, 5, and 8 of the repeating antigenic unit. In some embodiments, wherein a de-epitoped HMW glutenin having a mutation at positions 1 and 9, further comprises at least two additional substitution mutations at any of positions 3, 5, and 8, the substitution at position 3 is a polar amino acid or positively charged amino acid; or the substitution at position 5 is a hydrophobic amino acid; or the substitution at position 8 is to a small or aliphatic amino acid; or any combination thereof. In some embodiments, wherein a de-epitoped HMW glutenin comprises at least two additional substitution mutations at any of positions 3, 5, and 8, the substitution at position 3 is an arginine and the substitution at position 5 is leucine.

In some embodiments, a de-epitoped HMW glutenin as disclosed herein having a mutation at positions 1 and 9, comprises three additional substitution mutations at positions 3, 5 and 8, of the repeating antigenic unit. In some embodiments, a de-epitoped HMW glutenin having a mutation at positions 1 and 9, comprises three additional substitution mutations at positions 3, 5, and 8, wherein the substitution at position 3 is an arginine; or wherein the substitution at position 5 is a leucine; or wherein the substitution at position 8 is to a small or aliphatic amino acid; or any combination thereof. In some embodiments, a de-epitoped HMW glutenin comprising a mutation at positions 1 and 9, comprises at least three additional substitution mutations at positions 3, 5, and 8, wherein the substitution at position 3 is arginine and the substitution at position 5 is leucine.

In some embodiments, a de-epitoped HMW glutenin comprises a substitution mutation at position 9 of the repeating antigenic unit comprising a replacement of the amino acid residue at position 9 with an arginine, histidine, threonine, or a glutamate, or a combination thereof when more than one repeating antigenic unit is mutated. In some embodiments, a de-epitoped HMW glutenin comprises a substitution mutation at position 1 of the repeating antigenic unit comprising a replacement of the amino acid residue at position 1 with a histidine, a proline, a serine, an alanine, a lysine, or a glutamate, or a combination thereof when more than one repeating antigenic unit is mutated.

In some embodiments, the amino acid sequence of a de-epitoped HMW glutenin comprises the sequence set forth in any of SEQ ID NOs: 52, 102-107, 109-112, 114-116, 118-119, 126-134, 137-138, 140-142, 148-151, 155-168, and 170-172. In some embodiments, the amino acid sequence of a de-epitoped HMW glutenin comprising mutations within T-cell epitopes, comprises the sequence set forth in any of SEQ ID NOs: 52, 102-107, 109-112, 114-116, 118-119, 126-134, 137-138, 140-142, 148-151, 155-168, and 170-172. In some embodiments, the amino acid sequence of a de-epitoped HMW glutenin comprising deletions of T-cell epitopes, comprises the sequence set forth in any of SEQ ID NOs: 52, 102-107, 167-168, and 170-172. In some embodiments, the amino acid sequence of a de-epitoped HMW glutenin comprising substitution mutations within T-cell epitopes, comprises the sequence set forth in any of SEQ ID NOs: 52, 109-112, 114-116, 118-119, 126-134, 137-138, 140-142, 148-151, 155-166.

In some embodiments, the amino acid sequence of a de-epitoped HMW glutenin comprising mutations of repeating antigenic units or variations thereof, comprises the sequence set forth in any of SEQ ID NOs: 52, 102-107, 109-112, 114-116, 118-119, 126-134, 137-138, 140-142, 148-151, 155-168, and 170-172. In some embodiments, the amino acid sequence of a de-epitoped HMW glutenin comprising deletions of repeating antigenic units or variations thereof, comprises the sequence set forth in any of SEQ ID NOs: 52, 102-107, 167-168, and 170-172. In some embodiments, the amino acid sequence of a de-epitoped HMW glutenin comprising substitution mutations within of repeating antigenic units or variations thereof, comprises the sequence set forth in any of SEQ ID NOs: 109-112, 114-116, 118-119, 126-134, 137-138, 140-142, 148-151, and 155-166.

In some embodiments, the amino acid sequence of a de-epitoped HMW glutenin comprising substitution mutations comprises the sequence set forth in any of SEQ ID NOs: 38, 40-42, or 108-125. In some embodiments, the amino acid sequence of a de-epitoped HMW glutenin comprises the sequence set forth in any of SEQ ID NOs: 38, 40, or 41. In certain embodiments, the de-epitoped high molecular weight glutenin comprises at least one of the amino acid sequences set forth in SEQ ID NOs: 108-119. In certain embodiments, the de-epitoped high molecular weight glutenin comprises at least one of the amino acid sequences set forth in SEQ ID NOs: 109, 111, 112, 115, 116, 117, 118 or 119. In certain embodiments, the de-epitoped high molecular weight glutenin comprises at least one of the amino acid sequences set forth in SEQ ID NOs: 109, 111, 115, 116, 118 or 119. In certain embodiments, the de-epitoped high molecular weight glutenin comprises at least one of the amino acid sequences set forth in SEQ ID NOs: 109, 111 or 119. In certain embodiments, the amino acid sequences of de-epitoped HMW glutenins in which all of the repeating units are substituted are set forth in SEQ ID NOs: 126-142, 148-151, or 155-166. In some embodiments, the amino acid sequence of a de-epitoped HMW glutenin comprising substitution mutations comprises the sequence set forth in any of SEQ ID NOs: 126-142, 148-151, or 155-166. In some embodiments, the amino acid sequence of a de-epitoped HMW glutenin comprising deletion mutations comprises the sequence set forth in any of SEQ ID NOs: 52, 102-107, 167-168 or 170-172.

In some embodiments, a mutated antigenic unit comprises the sequence set forth in any of 52, 109-112, 114-116, or 118-119.

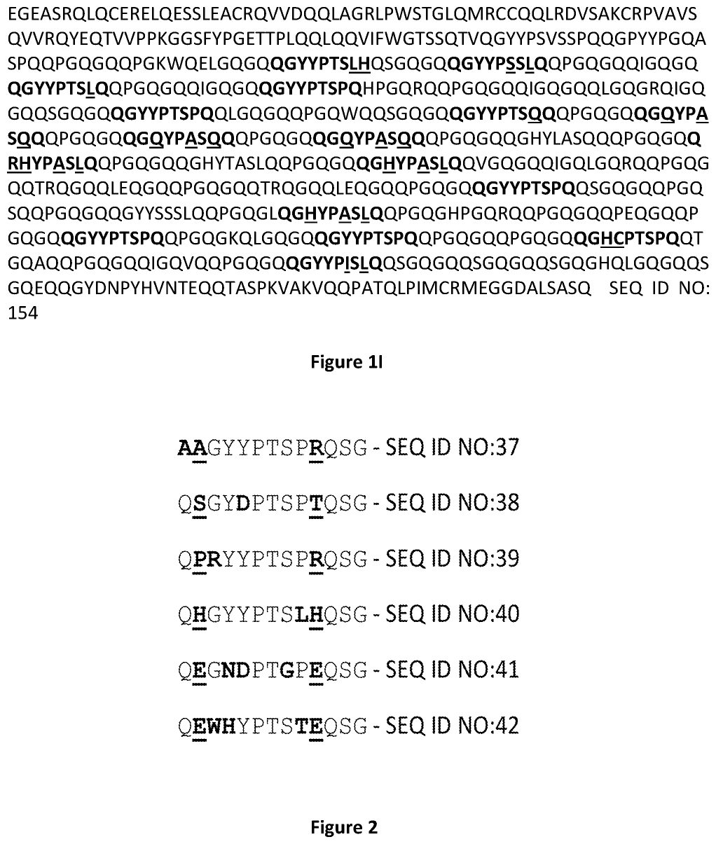

A non-limiting example of mutated 15-mer peptides comprising antigenic units is provided in FIG. 2, wherein position 1 and position 9 are mutated for each mutant antigenic unit shown.

A skilled artisan would appreciate that a de-epitoped HMW glutenin comprising mutated repeating antigenic units, may be characterized by the mutated repeating antigenic units, which in some embodiments comprise 9-mer to 15-mer peptides, or are a deletion of the repeating antigenic units. In some embodiments, a de-epitoped HMW glutenin comprising mutated repeating antigenic units, may be characterized by de-epitoped HMW glutenin partial sequences. Table 1 below sets forth de-epitoped HMW glutenin partial amino sequences and the corresponding mutated repeating antigenic units or deletion thereof, comprised within these de-epitoped HMW glutenin partial amino sequences.

TABLE 1
Sequences of a Subset of De-Epitoped HMW Glutenin
Partial Amino Acid Sequences and the Mutated
Repeating Units comprised Therein.

| SEQ ID NO: De-epitoped HMW | SEQ ID NO: of Mutated Repeating |
|---|---|
| Glutenin Partial Sequence | Antigenic Unit Comprised Therein |
| 102 | deletion of repeating antigenic units |
| 103 | deletion of repeating antigenic units |
| 104 | deletion of repeating antigenic units |
| 105 | deletion of repeating antigenic units |
| 106 | deletion of repeating antigenic units |
| 107 | deletion of repeating antigenic units |
| 126 | 111 |
| 127 | 119 |
| 128 | 111, 54 |
| 129 | 109, 52 |
| 130 | 52 |
| 131 | 109 |
| 132 | 118 |
| 133 | 115 |
| 134 | 118, 169 |
| 137 | 116 |
| 138 | 116, 145 |
| 140 | 115, 144 |
| 141 | 114 |
| 142 | 114, 143 |
| 148 | 52 |
| 149 | N/A |
| 150 | N/A |
| 151 | N/A |
| 155 | 109 |
| 156 | 109 |
| 158 | 111 |
| 159 | 52 |
| 160 | 52 |
| 161 | 52 |
| 162 | 52 |
| 163 | N/A |
| 164 | N/A |
| 165 | N/A |
| 166 | N/A |
| 167 | deletion of repeating antigenic units |
| 168 | deletion of repeating antigenic units |
| 170 | deletion of repeating antigenic units |
| 171 | deletion of repeating antigenic units |
| 172 | deletion of repeating antigenic units |

N/A—not applicable

In some embodiments, the amino acid sequence of a de-epitoped HMW glutenin comprises the sequence set forth in SEQ ID NO: 38. In some embodiments, the amino acid sequence of a de-epitoped HMW glutenin comprises the sequence set forth in SEQ ID NO: 40. In some embodiments, the amino acid sequence of a de-epitoped HMW glutenin comprises the sequence set forth in SEQ ID NO: 41. In some embodiments, the amino acid sequence of a de-epitoped HMW glutenin comprises the sequence set forth in SEQ ID NO: 42. In some embodiments, the amino acid sequence of a de-epitoped HMW glutenin comprises the sequence set forth in SEQ ID NO: 51. In some embodiments, the amino acid sequence of a de-epitoped HMW glutenin comprises the sequence set forth in SEQ ID NO: 52. In some embodiments, the amino acid sequence of a de-epitoped HMW glutenin comprises the sequence set forth in SEQ ID NO: 53. In some embodiments, the amino acid sequence of a de-epitoped HMW glutenin comprises the sequence set forth in SEQ ID NO: 54. In some embodiments, the amino acid sequence of a de-epitoped HMW glutenin comprises the sequence set forth in SEQ ID NO: 55. In some embodiments, the amino acid sequence of a de-epitoped HMW glutenin comprises the sequence set forth in SEQ ID NO: 56. In some embodiments, the amino acid sequence of a de-epitoped HMW glutenin comprises the sequence set forth in SEQ ID NO: 102. In some embodiments, the amino acid sequence of a de-epitoped HMW glutenin comprises the sequence set forth in SEQ ID NO: 103. In some embodiments, the amino acid sequence of a de-epitoped HMW glutenin comprises the sequence set forth in SEQ ID NO: 104. In some embodiments, the amino acid sequence of a de-epitoped HMW glutenin comprises the sequence set forth in SEQ ID NO: 105. In some embodiments, the amino acid sequence of a de-epitoped HMW glutenin comprises the sequence set forth in SEQ ID NO: 106. In some embodiments, the amino acid sequence of a de-epitoped HMW glutenin comprises the sequence set forth in SEQ ID NO: 107. In some embodiments, the amino acid sequence of a de-epitoped HMW glutenin comprises the sequence set forth in SEQ ID NO: 108. In some embodiments, the amino acid sequence of a de-epitoped HMW glutenin comprises the sequence set forth in SEQ ID NO: 109. In some embodiments, the amino acid sequence of a de-epitoped HMW glutenin comprises the sequence set forth in SEQ ID NO: 110. In some embodiments, the amino acid sequence of a de-epitoped HMW glutenin comprises the sequence set forth in SEQ ID NO: 111. In some embodiments, the amino acid sequence of a de-epitoped HMW glutenin comprises the sequence set forth in SEQ ID NO: 112. In some embodiments, the amino acid sequence of a de-epitoped HMW glutenin comprises the sequence set forth in SEQ ID NO: 113. In some embodiments, the amino acid sequence of a de-epitoped HMW glutenin comprises the sequence set forth in SEQ ID NO: 114. In some embodiments, the amino acid sequence of a de-epitoped HMW glutenin comprises the sequence set forth in SEQ ID NO: 115. In some embodiments, the amino acid sequence of a de-epitoped HMW glutenin comprises the sequence set forth in SEQ ID NO: 116. In some embodiments, the amino acid sequence of a de-epitoped HMW glutenin comprises the sequence set forth in SEQ ID NO: 117. In some embodiments, the amino acid sequence of a de-epitoped HMW glutenin comprises the sequence set forth in SEQ ID NO: 118. In some embodiments, the amino acid sequence of a de-epitoped HMW glutenin comprises the sequence set forth in SEQ ID NO: 119. In some embodiments, the amino acid sequence of a de-epitoped HMW glutenin comprises the sequence set forth in SEQ ID NO: 120. In some embodiments, the amino acid sequence of a de-epitoped HMW glutenin comprises the sequence set forth in SEQ ID NO: 121. In some embodiments, the amino acid sequence of a de-epitoped HMW glutenin comprises the sequence set forth in SEQ ID NO: 122. In some embodiments, the amino acid sequence of a de-epitoped HMW glutenin comprises the sequence set forth in SEQ ID NO: 123. In some embodiments, the amino acid sequence of a de-epitoped HMW glutenin comprises the sequence set forth in SEQ ID NO: 124. In some embodiments, the amino acid sequence of a de-epitoped HMW glutenin comprises the sequence set forth in SEQ ID NO: 125. In some embodiments, the amino acid sequence of a de-epitoped HMW glutenin comprises the sequence set forth in SEQ ID NO: 126. In some embodiments, the amino acid sequence of a de-epitoped HMW glutenin comprises the sequence set forth in SEQ ID NO: 127. In some embodiments, the amino acid sequence of a de-epitoped HMW glutenin comprises the sequence set forth in SEQ ID NO: 128. In some embodiments, the amino acid sequence of a de-epitoped HMW glutenin comprises the sequence set forth in SEQ ID NO: 129. In some embodiments, the amino acid sequence of a de-epitoped HMW glutenin comprises the sequence set forth in SEQ ID NO: 130. In some embodiments, the amino acid sequence of a de-epitoped HMW glutenin comprises the sequence set forth in SEQ ID NO: 131. In some embodiments, the amino acid sequence of a de-epitoped HMW glutenin comprises the sequence set forth in SEQ ID NO: 132. In some embodiments, the amino acid sequence of a de-epitoped HMW glutenin comprises the sequence set forth in SEQ ID NO: 133. In some embodiments, the amino acid sequence of a de-epitoped HMW glutenin comprises the sequence set forth in SEQ ID NO: 134. In some embodiments, the amino acid sequence of a de-epitoped HMW glutenin comprises the sequence set forth in SEQ ID NO: 135. In some embodiments, the amino acid sequence of a de-epitoped HMW glutenin comprises the sequence set forth in SEQ ID NO: 136. In some embodiments, the amino acid sequence of a de-epitoped HMW glutenin comprises the sequence set forth in SEQ ID NO: 137. In some embodiments, the amino acid sequence of a de-epitoped HMW glutenin comprises the sequence set forth in SEQ ID NO: 138. In some embodiments, the amino acid sequence of a de-epitoped HMW glutenin comprises the sequence set forth in SEQ ID NO: 140. In some embodiments, the amino acid sequence of a de-epitoped HMW glutenin comprises the sequence set forth in SEQ ID NO: 141. In some embodiments, the amino acid sequence of a de-epitoped HMW glutenin comprises the sequence set forth in SEQ ID NO: 142. In some embodiments, the amino acid sequence of a de-epitoped HMW glutenin comprises the sequence set forth in SEQ ID NO: 143. In some embodiments, the amino acid sequence of a de-epitoped HMW glutenin comprises the sequence set forth in SEQ ID NO: 144. In some embodiments, the amino acid sequence of a de-epitoped HMW glutenin comprises the sequence set forth in SEQ ID NO: 145. In some embodiments, the amino acid sequence of a de-epitoped HMW glutenin comprises the sequence set forth in SEQ ID NO: 146. In some embodiments, the amino acid sequence of a de-epitoped HMW glutenin comprises the sequence set forth in SEQ ID NO: 147. In some embodiments, the amino acid sequence of a de-epitoped HMW glutenin comprises the sequence set forth in SEQ ID NO: 148. In some embodiments, the amino acid sequence of a de-epitoped HMW glutenin comprises the sequence set forth in SEQ ID NO: 149. In some embodiments, the amino acid sequence of a de-epitoped HMW glutenin comprises the sequence set forth in SEQ ID NO: 150. In some embodiments, the amino acid sequence of a de-epitoped HMW glutenin comprises the sequence set forth in SEQ ID NO: 151. In some embodiments, the amino acid sequence of a de-epitoped HMW glutenin comprises the sequence set forth in SEQ ID NO: 155. In some embodiments, the amino acid sequence of a de-epitoped HMW glutenin comprises the sequence set forth in SEQ ID NO: 156. In some embodiments, the amino acid sequence of a de-epitoped HMW glutenin comprises the sequence set forth in SEQ ID NO: 157. In some embodiments, the amino acid sequence of a de-epitoped HMW glutenin comprises the sequence set forth in SEQ ID NO: 158. In some embodiments, the amino acid sequence of a de-epitoped HMW glutenin comprises the sequence set forth in SEQ ID NO: 159. In some embodiments, the amino acid sequence of a de-epitoped HMW glutenin comprises the sequence set forth in SEQ ID NO: 160. In some embodiments, the amino acid sequence of a de-epitoped HMW glutenin comprises the sequence set forth in SEQ ID NO: 161. In some embodiments, the amino acid sequence of a de-epitoped HMW glutenin comprises the sequence set forth in SEQ ID NO: 162. In some embodiments, the amino acid sequence of a de-epitoped HMW glutenin comprises the sequence set forth in SEQ ID NO: 163. In some embodiments, the amino acid sequence of a de-epitoped HMW glutenin comprises the sequence set forth in SEQ ID NO: 164. In some embodiments, the amino acid sequence of a de-epitoped HMW glutenin comprises the sequence set forth in SEQ ID NO: 165. In some embodiments, the amino acid sequence of a de-epitoped HMW glutenin comprises the sequence set forth in SEQ ID NO: 166. In some embodiments, the amino acid sequence of a de-epitoped HMW glutenin comprises the sequence set forth in SEQ ID NO: 167. In some embodiments, the amino acid sequence of a de-epitoped HMW glutenin comprises the sequence set forth in SEQ ID NO: 168. In some embodiments, the amino acid sequence of a de-epitoped HMW glutenin comprises the sequence set forth in SEQ ID NO: 169. In some embodiments, the amino acid sequence of a de-epitoped HMW glutenin comprises the sequence set forth in SEQ ID NO: 170. In some embodiments, the amino acid sequence of a de-epitoped HMW glutenin comprises the sequence set forth in SEQ ID NO: 171. In some embodiments, the amino acid sequence of a de-epitoped HMW glutenin comprises the sequence set forth in SEQ ID NO: 172. In some embodiments, the amino acid sequence of a de-epitoped HMW glutenin comprises the sequence set forth in SEQ ID NO: 174.

One skilled in the art would appreciate that a de-epitoped HMW glutenin disclosed herein may comprise different combinations of the above disclosed sequences, for example different combinations of mutated repeating antigenic units. Thus, for example, a single de-epitoped high molecular weight glutenin may in some embodiments, comprise particular combinations of SEQ ID NO: 38, 40-42 and 108-119, and/or various copy numbers of any one of the above disclosed sequences.

In other embodiments, each of the repeating antigenic units comprising nine amino acids in a modified HMW glutenin is replaced with mutated antigenic units comprising identical mutations, which may be substitution mutations within the antigenic unit or deletion mutations of the antigenic unit.

According to some embodiments, a de-epitoped HMW glutenin does not comprise any of the sequences as set forth in SEQ ID NOs: 57-69.

In some embodiments, the mutations present in a de-epitoped HMW glutenin described herein, do not negatively affect the function of the polypeptide (e.g. for a modified wheat HMW glutenin, the mutations do not affect the ability of the wheat polypeptide to generate a dough with elastic properties).

In one embodiment, the term “mutating” may encompass expressing a recombinant polypeptide that has a mutation with respect to the wild-type (WT) protein, for example a WT HMW glutenin. In some embodiments, a de-epitoped HMW glutenin polypeptide disclosed herein is a recombinant polypeptide.

In some embodiments, a de-epitoped HMW glutenin disclosed herein, does not comprise an antigenic unit that binds to MHC class DQ2 or DQ8 with an IC50 of less than 30 PM. In some embodiments, a de-epitoped HMW glutenin disclosed herein, does not comprise a repeating motif that binds to MHC class DQ2 or DQ8 with an IC50 of less than 30 μm. In some embodiments, a de-epitoped HMW glutenin disclosed herein, does not comprise a CD-relevant epitope that binds to MHC class DQ2 or DQ8 with an IC50 of less than 30 μM. In some embodiments, peptides derived from a de-epitoped HMW glutenin disclosed herein, do not bind to MHC class DQ2 or DQ8 with an IC50 of less than 30 μM.

In some embodiments, a de-epitoped HMW glutenin disclosed herein, does not comprise an antigenic unit that binds to MHC class DQ2 or DQ8 with an IC50 of less than 251M. In some embodiments, a de-epitoped HMW glutenin disclosed herein, does not comprise a repeating motif that binds to MHC class DQ2 or DQ8 with an IC50 of less than 25 μm. In some embodiments, a de-epitoped HMW glutenin disclosed herein, does not comprise a CD-relevant epitope that binds to MHC class DQ2 or DQ8 with an IC50 of less than 25 μM. In some embodiments, peptides derived from a de-epitoped HMW glutenin disclosed herein, do not bind to MHC class DQ2 or DQ8 with an IC50 of less than 25 μM.

In some embodiments, a de-epitoped HMW glutenin disclosed herein, does not comprise an antigenic unit that binds to MHC class DQ2 or DQ8 with an IC50 of less than 20 PM. In some embodiments, a de-epitoped HMW glutenin disclosed herein, does not comprise a repeating motif that binds to MHC class DQ2 or DQ8 with an IC50 of less than 20 μm. In some embodiments, a de-epitoped HMW glutenin disclosed herein, does not comprise a CD-relevant epitope that binds to MHC class DQ2 or DQ8 with an IC50 of less than 20 μM. In some embodiments, peptides derived from a de-epitoped HMW glutenin disclosed herein, do not bind to MHC class DQ2 or DQ8 with an IC50 of less than 20 μM.

### Nucleotides, Vectors, and Host Cells

Disclosed herein are isolated polynucleotides, which encode any of the above described de-epitoped HMW glutenin polypeptides. Such polynucleotides may be used to express the above described de-epitoped HMW glutenin polypeptides in host cells (e.g. bacteria, plants, yeast, or mammalian cell hosts).

In some embodiments, isolated polynucleotides encoding the de-epitoped HMW glutenin are disclosed herein.

The term “nucleotide”, “nucleotide sequence”, “polynucleotide”, “polynucleotide sequence”, or “nucleic acid molecule” or the like, as used herein may encompass DNA molecules and RNA molecules or modified RNA molecules. A nucleic acid molecule may be single-stranded or double-stranded. In some embodiments, a nucleotide comprises a modified nucleotide. In some embodiments, a nucleotide comprises an mRNA. In some embodiments, a nucleotide comprises a modified mRNA. In some embodiments, a nucleotide comprises a modified mRNA, wherein the modified mRNA comprises a 5′-capped mRNA. In some embodiments, a modified mRNA comprises a molecule in which some of the nucleosides have been replaced by either naturally modified or synthetic nucleosides. In some embodiments, a modified nucleotide comprises a modified mRNA comprising a 5′-capped mRNA and wherein some of the nucleosides have been replaced by either naturally modified or synthetic nucleosides.

A skilled artisan would appreciate that the terms “polynucleotide”, “nucleic acid sequence”, “nucleic acid”, and variations thereof may be generic encompassing polydeoxyribonucleotides (containing 2-deoxy-D-ribose), to polyribonucleotides (containing D-ribose), to any other type of polynucleotide that is an N-glycoside of a purine or pyrimidine base, and to other polymers containing non-nucleotidic backbones, provided that the polymers contain nucleobases in a configuration that allows for base pairing and base stacking, as found in DNA and RNA. Thus, these terms include known types of nucleic acid sequence modifications, for example, substitution of one or more of the naturally occurring nucleotides with an analog, and inter-nucleotide modifications.

In some embodiments, an isolated polynucleotide encoding a HMW glutenin comprise a nucleotide sequence which is essentially free of other genomic nucleotide sequences that naturally flank the nucleic acid in genomic DNA.

Disclosed herein, in some embodiments, are nucleotide or nucleic acid sequences encoding any of the HMW glutenin molecules disclosed herein.

Methods of introducing nucleic acid alterations to a gene of interest are well known in the art [see for example Menke D. Genesis (2013) 51: —618; Capecchi, Science (1989) 244:1288-1292; Santiago et al. Proc Natl Acad Sci USA (2008) 105:5809-5814; International Patent Application Nos. WO 2014085593, WO 2009071334 and WO 2011146121; U.S. Pat. Nos. 8,771,945, 8,586,526, 6,774,279 and UP Patent Application Publication Nos. 20030232410, 20050026157, US20060014264; the contents of which are incorporated by reference in their entireties] and include targeted homologous recombination, site specific recombinases, PB transposases and genome editing by engineered nucleases. Agents for introducing nucleic acid alterations to a gene of interest can be designed by publicly available sources or obtained commercially from Transposagen, Addgene and Sangamo Biosciences. In some embodiments, the generation of the alterations in the sequences of the genes may be achieved by screening sequences of existing plants in search of an existing variant of the desired sequence. Then, this existing sequence can be introduced into the genome of the target genome by crossbreeding, or by gene editing. In other embodiments the desired variations will be introduced by introducing random mutagenesis, followed by screening for variants where the desired mutations occurred, followed by crossbreeding.

Following is a description of various exemplary methods used to introduce nucleic acid alterations to a gene of interest and agents for implementing same that can be used according to specific embodiments disclosed herein.

Genome editing using engineered endonucleases—this approach refers to a reverse genetics method using artificially engineered nucleases to cut and create specific double-stranded breaks at a desired location(s) in the genome, which are then repaired by cellular endogenous processes such as, homology directed repair (HDS) and non-homologous end-joining (NHEJ). NHEJ directly joins the DNA ends in a double-stranded break, while HDR utilizes a homologous sequence as a template for regenerating the missing DNA sequence at the break point. In order to introduce specific nucleotide modifications to the genomic DNA, a DNA repair template containing the desired sequence must be present during HDR. Genome editing cannot be performed using traditional restriction endonucleases since most restriction enzymes recognize a few base pairs on the DNA as their target and the probability is very high that the recognized base pair combination will be found in many locations across the genome resulting in multiple cuts not limited to a desired location. To overcome this challenge and create site-specific single- or double-stranded breaks, several distinct classes of nucleases have been discovered and bioengineered to date. These include the meganucleases, Zinc finger nucleases (ZFNs), transcription-activator like effector nucleases (TALENs) and CRISPR/Cas system.

Meganucleases—Meganucleases are commonly grouped into four families: the LAGLIDADG (SEQ ID NO: 36) family, the GIY-YIG (SEQ ID NO: 70) family, the His-Cys box family and the HNH family. These families are characterized by structural motifs, which affect catalytic activity and recognition sequence. For instance, members of the LAGLIDADG (SEQ ID NO: 36) family are characterized by having either one or two copies of the conserved LAGLIDADG (SEQ ID NO: 36) motif. The four families of meganucleases are widely separated from one another with respect to conserved structural elements and, consequently, DNA recognition sequence specificity and catalytic activity. Meganucleases are found commonly in microbial species and have the unique property of having very long recognition sequences (>14 bp) thus making them naturally very specific for cutting at a desired location. This can be exploited to make site-specific double-stranded breaks in genome editing. One of skill in the art can use these naturally occurring meganucleases, however the number of such naturally occurring meganucleases is limited. To overcome this challenge, mutagenesis and high throughput screening methods have been used to create meganuclease variants that recognize unique sequences. For example, various meganucleases have been fused to create hybrid enzymes that recognize a new sequence. Alternatively, DNA interacting amino acids of the meganuclease can be altered to design sequence specific meganucleases (see e.g., U.S. Pat. No. 8,021,867). Meganucleases can be designed using the methods described in e.g., Certo, M T et al. Nature Methods (2012) 9:073-975; U.S. Pat. Nos. 8,304,222; 8,021,867; 8,119,381; 8, 124,369; 8, 129,134; 8,133,697; 8,143,015; 8,143,016; 8, 148,098; or 8, 163,514, the contents of each are incorporated herein by reference in their entirety. Alternatively, meganucleases with site specific cutting characteristics can be obtained using commercially available technologies e.g., Precision Biosciences' Directed Nuclease Editor™ genome editing technology.

ZFNs and TALENs—Two distinct classes of engineered nucleases, zinc-finger nucleases (ZFNs) and transcription activator-like effector nucleases (TALENs), have both proven to be effective at producing targeted double-stranded breaks (Christian et al., 2010; Kim et al., 1996; Li et al., 2011; Mahfouz et al., 2011; Miller et al., 2010).

Basically, ZFNs and TALENs restriction endonuclease technology utilizes a non-specific DNA cutting enzyme which is linked to a specific DNA binding domain (either a series of zinc finger domains or TALE repeats, respectively). Typically, a restriction enzyme whose DNA recognition site and cleaving site are separate from each other is selected. The cleaving portion is separated and then linked to a DNA binding domain, thereby yielding an endonuclease with very high specificity for a desired sequence. An exemplary restriction enzyme with such properties is Fokl. Additionally, Fokl has the advantage of requiring dimerization to have nuclease activity and this means the specificity increases dramatically as each nuclease partner recognizes a unique DNA sequence. To enhance this effect, Fokl nucleases have been engineered that can only function as heterodimers and have increased catalytic activity. The heterodimer functioning nucleases avoid the possibility of unwanted homodimer activity and thus increase specificity of the double-stranded break.

Thus, for example to target a specific site, ZFNs and TALENs are constructed as nuclease pairs, with each member of the pair designed to bind adjacent sequences at the targeted site. Upon transient expression in cells, the nucleases bind to their target sites and the FokI domains heterodimerize to create a double-stranded break. Repair of these double-stranded breaks through the nonhomologous end-joining (NHEJ) pathway most often results in small deletions or small sequence insertions. Since each repair made by NHEJ is unique, the use of a single nuclease pair can produce an allelic series with a range of different deletions at the target site. The deletions typically range anywhere from a few base pairs to a few hundred base pairs in length, but larger deletions have successfully been generated in cell culture by using two pairs of nucleases simultaneously (Carlson et al., 2012; Lee et al., 2010). In addition, when a fragment of DNA with homology to the targeted region is introduced in conjunction with the nuclease pair, the double-stranded break can be repaired via homology directed repair to generate specific modifications (Li et al., 2011; Miller et al., 2010; Urnov et al., 2005).

Although the nuclease portions of both ZFNs and TALENs have similar properties, the difference between these engineered nucleases is in their DNA recognition peptide. ZFNs rely on Cys2—His2 zinc fingers and TALENs on TALEs. Both of these DNA recognizing peptide domains have the characteristic that they are naturally found in combinations in their proteins. Cys2-His2 Zinc fingers are typically found in repeats that are 3 bp apart and are found in diverse combinations in a variety of nucleic acid interacting proteins. TALEs on the other hand are found in repeats with a one-to-one recognition ratio between the amino acids and the recognized nucleotide pairs. Because both zinc fingers and TALEs happen in repeated patterns, different combinations can be tried to create a wide variety of sequence specificities. Approaches for making site-specific zinc finger endonucleases include, e.g., modular assembly (where Zinc fingers correlated with a triplet sequence are attached in a row to cover the required sequence), OPEN (low-stringency selection of peptide domains vs. triplet nucleotides followed by high-stringency selections of peptide combination vs. the final target in bacterial systems), and bacterial one-hybrid screening of zinc finger libraries, among others. ZFNs can also be designed and obtained commercially from e.g., Sangamo Biosciences™ (Richmond, CA).

Method for designing and obtaining TALENs are described in e.g. Reyon et al. Nature Biotechnology 2012 May; 30(5):460-5; Miller et al. Nat Biotechnol. (2011) 29: 143-148; Cermak et al. Nucleic Acids Research (2011) 39 (12): e82 and Zhang et al. Nature Biotechnology (2011) 29 (2): 149-53. A recently developed web-based program named Mojo Hand was introduced by Mayo Clinic for designing TAL and TALEN constructs for genome editing applications (can be accessed through www(dot)talendesign(dot)org). TALEN can also be designed and obtained commercially from e.g., Sangamo Biosciences™ (Richmond, CA).

CRISPR-Cas system—Many bacteria and archea contain endogenous RNA-based adaptive immune systems that can degrade nucleic acids of invading phages and plasmids. These systems consist of clustered regularly interspaced short palindromic repeat (CRISPR) genes that produce RNA components and CRISPR associated (Cas) genes that encode protein components. The CRISPR RNAs (crRNAs) contain short stretches of homology to specific viruses and plasmids and act as guides to direct Cas nucleases to degrade the complementary nucleic acids of the corresponding pathogen. Studies of the type II CRISPR/Cas system of Streptococcus pyogenes have shown that three components form an RNA/protein complex and together are sufficient for sequence-specific nuclease activity: the Cas9 nuclease, a crRNA containing 20 base pairs of homology to the target sequence, and a trans-activating crRNA (tracrRNA) (Jinek et al. Science (2012) 337: 816-821.). It was further demonstrated that a synthetic chimeric guide RNA (gRNA) composed of a fusion between crRNA and tracrRNA could direct Cas9 to cleave DNA targets that are complementary to the crRNA in vitro. It was also demonstrated that transient expression of Cas9 in conjunction with synthetic gRNAs can be used to produce targeted double-stranded brakes in a variety of different species (Cho et al., 2013; Cong et al., 2013; DiCarlo et al., 2013; Hwang et al., 2013a,b; Jinek et al., 2013; Mali et al., 2013).

The CRIPSR/Cas system for genome editing contains two distinct components: a gRNA and an endonuclease e.g. Cas9.

The gRNA is typically a 20 nucleotide sequence encoding a combination of the target homologous sequence (crRNA) and the endogenous bacterial RNA that links the crRNA to the Cas9 nuclease (tracrRNA) in a single chimeric transcript. The gRNA/Cas9 complex is recruited to the target sequence by the base-pairing between the gRNA sequence and the complement genomic DNA. For successful binding of Cas9, the genomic target sequence must also contain the correct Protospacer Adjacent Motif (PAM) sequence immediately following the target sequence. The binding of the gRNA/Cas9 complex localizes the Cas9 to the genomic target sequence so that the Cas9 can cut both strands of the DNA causing a double-strand break. Just as with ZFNs and TALENs, the double-stranded brakes produced by CRISPR/Cas can undergo homologous recombination or NHEJ.

The Cas9 nuclease has two functional domains: RuvC and HNH, each cutting a different DNA strand. When both of these domains are active, the Cas9 causes double strand breaks in the genomic DNA.

A significant advantage of CRISPR/Cas is that the high efficiency of this system coupled with the ability to easily create synthetic gRNAs enables multiple genes to be targeted simultaneously. In addition, the majority of cells carrying the mutation present biallelic mutations in the targeted genes.

However, apparent flexibility in the base-pairing interactions between the gRNA sequence and the genomic DNA target sequence allows imperfect matches to the target sequence to be cut by Cas9.

Modified versions of the Cas9 enzyme containing a single inactive catalytic domain, either RuvC- or HNH-, are called ‘nickases’. With only one active nuclease domain, the Cas9 nickase cuts only one strand of the target DNA, creating a single-strand break or ‘nick’. A single-strand break, or nick, is normally quickly repaired through the HDR pathway, using the intact complementary DNA strand as the template. However, two proximal, opposite strand nicks introduced by a Cas9 nickase are treated as a double-strand break, in what is often referred to as a ‘double nick’ CRISPR system. A double-nick can be repaired by either NHEJ or HDR depending on the desired effect on the gene target. Thus, if specificity and reduced off-target effects are crucial, using the Cas9 nickase to create a double-nick by designing two gRNAs with target sequences in close proximity and on opposite strands of the genomic DNA would decrease off-target effect as either gRNA alone will result in nicks that will not change the genomic DNA.

Modified versions of the Cas9 enzyme containing two inactive catalytic domains (dead Cas9, or dCas9) have no nuclease activity while still able to bind to DNA based on gRNA specificity. The dCas9 can be utilized as a platform for DNA transcriptional regulators to activate or repress gene expression by fusing the inactive enzyme to known regulatory domains. For example, the binding of dCas9 alone to a target sequence in genomic DNA can interfere with gene transcription.

There are a number of publicly available tools available to help choose and/or design target sequences as well as lists of bioinformatically determined unique gRNAs for different genes in different species such as the Feng Zhang lab's Target Finder, the Michael Boutros lab's Target Finder (E-CRISP), the RGEN Tools: Cas-OFFinder, the CasFinder: Flexible algorithm for identifying specific Cas9 targets in genomes and the CRISPR Optimal Target Finder.

In order to use the CRISPR system, both gRNA and Cas9 should be expressed in a target cell. The insertion vector can contain both cassettes on a single plasmid or the cassettes are expressed from two separate plasmids. CRISPR plasmids are commercially available such as the px330 plasmid from Addgene.

“Hit and run” or “in-out”—involves a two-step recombination procedure. In the first step, an insertion-type vector containing a dual positive/negative selectable marker cassette is used to introduce the desired sequence alteration. The insertion vector contains a single continuous region of homology to the targeted locus and is modified to carry the mutation of interest. This targeting construct is linearized with a restriction enzyme at one site within the region of homology, electroporated into the cells, and positive selection is performed to isolate homologous recombinants. These homologous recombinants contain a local duplication that is separated by intervening vector sequence, including the selection cassette. In the second step, targeted clones are subjected to negative selection to identify cells that have lost the selection cassette via intrachromosomal recombination between the duplicated sequences. The local recombination event removes the duplication and, depending on the site of recombination, the allele either retains the introduced mutation or reverts to wild type. The end result is the introduction of the desired modification without the retention of any exogenous sequences.

The “double-replacement” or “tag and exchange” strategy—involves a two-step selection procedure similar to the hit and run approach, but requires the use of two different targeting constructs. In the first step, a standard targeting vector with 3′ and 5′ homology arms is used to insert a dual positive/negative selectable cassette near the location where the mutation is to be introduced. After electroporation and positive selection, homologously targeted clones are identified. Next, a second targeting vector that contains a region of homology with the desired mutation is electroporated into targeted clones, and negative selection is applied to remove the selection cassette and introduce the mutation. The final allele contains the desired mutation while eliminating unwanted exogenous sequences.

Site-Specific Recombinases—The Cre recombinase derived from the P1 bacteriophage and Flp recombinase derived from the yeast Saccharomyces cerevisiae are site-specific DNA recombinases each recognizing a unique 34 base pair DNA sequence (termed “Lox” and “FRT”, respectively) and sequences that are flanked with either Lox sites or FRT sites can be readily removed via site-specific recombination upon expression of Cre or Flp recombinase, respectively. For example, the Lox sequence is composed of an asymmetric eight base pair spacer region flanked by 13 base pair inverted repeats. Cre recombines the 34 base pair lox DNA sequence by binding to the 13 base pair inverted repeats and catalyzing strand cleavage and religation within the spacer region. The staggered DNA cuts made by Cre in the spacer region are separated by 6 base pairs to give an overlap region that acts as a homology sensor to ensure that only recombination sites having the same overlap region recombine.

Basically, the site specific recombinase system offers means for the removal of selection cassettes after homologous recombination. This system also allows for the generation of conditional altered alleles that can be inactivated or activated in a temporal or tissue-specific manner. Of note, the Cre and Flp recombinases leave behind a Lox or FRT “scar” of 34 base pairs. The Lox or FRT sites that remain are typically left behind in an intron or 3′ UTR of the modified locus, and current evidence suggests that these sites usually do not interfere significantly with gene function.

Thus, Cre/Lox and Flp/FRT recombination involves introduction of a targeting vector with 3′ and 5′ homology arms containing the mutation of interest, two Lox or FRT sequences and typically a selectable cassette placed between the two Lox or FRT sequences. Positive selection is applied and homologous recombinants that contain targeted mutation are identified. Transient expression of Cre or Flp in conjunction with negative selection results in the excision of the selection cassette and selects for cells where the cassette has been lost. The final targeted allele contains the Lox or FRT scar of exogenous sequences.

Transposases—As used herein, the term “transposase” refers to an enzyme that binds to the ends of a transposon and catalyzes the movement of the transposon to another part of the genome.

As used herein the term “transposon” refers to a mobile genetic element comprising a nucleotide sequence which can move around to different positions within the genome of a single cell. In the process the transposon can cause mutations and/or change the amount of a DNA in the genome of the cell.

A number of transposon systems that are able to also transpose in cells e.g. vertebrates have been isolated or designed, such as Sleeping Beauty [Izsvák and Ivics Molecular Therapy (2004) 9, 147-156], piggyBac [Wilson et al. Molecular Therapy (2007) 15, 139-145], Tol2 [Kawakami et al. PNAS (2000) 97 (21): 11403-11408] or Frog Prince [Miskey et al. Nucleic Acids Res. Dec. 1, (2003) 31(23): 6873-6881]. Generally, DNA transposons translocate from one DNA site to another in a simple, cut-and-paste manner. Each of these elements has their own advantages, for example, Sleeping Beauty is particularly useful in region-specific mutagenesis, whereas Tol2 has the highest tendency to integrate into expressed genes. Hyperactive systems are available for Sleeping Beauty and piggyBac. Most importantly, these transposons have distinct target site preferences, and can therefore introduce sequence alterations in overlapping, but distinct sets of genes. Therefore, to achieve the best possible coverage of genes, the use of more than one element is particularly preferred. The basic mechanism is shared between the different transposases, therefore we will describe piggyBac (PB) as an example.

PB is a 2.5 kb insect transposon originally isolated from the cabbage looper moth, Trichoplusia ni. The PB transposon consists of asymmetric terminal repeat sequences that flank a transposase, PBase. PBase recognizes the terminal repeats and induces transposition via a “cut-and-paste” based mechanism, and preferentially transposes into the host genome at the tetranucleotide sequence TTAA. Upon insertion, the TTAA target site is duplicated such that the PB transposon is flanked by this tetranucleotide sequence. When mobilized, PB typically excises itself precisely to reestablish a single TTAA site, thereby restoring the host sequence to its pretransposon state. After excision, PB can transpose into a new location or be permanently lost from the genome.

Typically, the transposase system offers an alternative means for the removal of selection cassettes after homologous recombination quite similar to the use Cre/Lox or Flp/FRT. Thus, for example, the PB transposase system involves introduction of a targeting vector with 3′ and 5′ homology arms containing the mutation of interest, two PB terminal repeat sequences at the site of an endogenous TTAA sequence and a selection cassette placed between PB terminal repeat sequences. Positive selection is applied and homologous recombinants that contain targeted mutation are identified. Transient expression of PBase removes in conjunction with negative selection results in the excision of the selection cassette and selects for cells where the cassette has been lost. The final targeted allele contains the introduced mutation with no exogenous sequences.

For PB to be useful for the introduction of sequence alterations, there must be a native TTAA site in relatively close proximity to the location where a particular mutation is to be inserted.

Genome editing using recombinant adeno-associated virus (rAAV) platform—this genome-editing platform is based on rAAV vectors which enable insertion, deletion or substitution of DNA sequences in the genomes of live mammalian cells. The rAAV genome is a single-stranded deoxyribonucleic acid (ssDNA) molecule, either positive- or negative-sensed, which is about 4.7 kb long. These single-stranded DNA viral vectors have high transduction rates and have a unique property of stimulating endogenous homologous recombination in the absence of double-strand DNA breaks in the genome. One of skill in the art can design a rAAV vector to target a desired genomic locus and perform both gross and/or subtle endogenous gene alterations in a cell. rAAV genome editing has the advantage in that it targets a single allele and does not result in any off-target genomic alterations. rAAV genome editing technology is commercially available, for example, the rAAV GENESIS™ system from Horizon™ (Cambridge, UK).

Methods for qualifying efficacy and detecting sequence alteration are well known in the art and include, but not limited to, DNA sequencing, electrophoresis, an enzyme-based mismatch detection assay and a hybridization assay such as PCR, RT-PCR, RNase protection, in-situ hybridization, primer extension, Southern blot, Northern Blot and dot blot analysis.

Sequence alterations in a specific gene can also be determined at the protein level using e.g. chromatography, electrophoretic methods, immunodetection assays such as ELISA and western blot analysis and immunohistochemistry.

In addition, one ordinarily skilled in the art can readily design a knock-in/knock-out construct including positive and/or negative selection markers for efficiently selecting transformed cells that underwent a homologous recombination event with the construct. Positive selection provides a means to enrich the population of clones that have taken up foreign DNA. Non-limiting examples of such positive markers include glutamine synthetase, dihydrofolate reductase (DHFR), markers that confer antibiotic resistance, such as neomycin, hygromycin, puromycin, and blasticidin S resistance cassettes. Negative selection markers are necessary to select against random integrations and/or elimination of a marker sequence (e.g. positive marker). Non-limiting examples of such negative markers include the herpes simplex-thymidine kinase (HSV-TK) which converts ganciclovir (GCV) into a cytotoxic nucleoside analog, hypoxanthine phosphoribosyltransferase (HPRT) and adenine phosphoribosytransferase (ARPT).

In some embodiments, disclosed herein is an expression vector comprising the isolated polynucleotide comprising any of the de-epitoped HMW glutenin modified proteins disclosed herein, operatively linked to a transcriptional regulatory sequence so as to allow expression of said de-epitoped HMW glutenin in a plant cell. In some embodiments, disclosed herein are host cells comprising the expression vector comprising the isolated polynucleotide comprising any of the de-epitoped HMW glutenin modified proteins disclosed herein. In some embodiments, the host cell comprises a plant cell. In some embodiments, a plant cell comprises a wheat cell. In some embodiments, a plant cell comprises a corn cell. In some embodiments, a plant cell comprises a tobacco cell. In some embodiments, the host cell comprises a yeast cell. In some embodiments, the host cell comprises a bacterial cell. In some embodiments, the host cell comprises a mammalian cell.

In some embodiments, a plant cell comprises a de-epitoped HMW glutenin. In some embodiments, a plant cell comprises a de-epitoped HMW glutenin, wherein the de-epitoped HMW glutenin is from the same species of plant compared with the plant cell. In some embodiments, a plant cell comprises a de-epitoped HMW glutenin, wherein the de-epitoped HMW glutenin is from a heterologous species of plant compared with the plant cell.

In some embodiments, a plant cell comprises a de-epitoped HMW glutenin. In some embodiments, a bacterial cell comprises a de-epitoped HMW glutenin. In some embodiments, a yeast cell comprises a de-epitoped HMW glutenin. In some embodiments, a mammalian cell comprises a de-epitoped HMW glutenin.

In some embodiments, a bacterial cell comprises a polynucleotide encoding a de-epitoped HMW glutenin. In some embodiments, a bacterial cell comprises a polynucleotide encoding a de-epitoped HMW glutenin. In some embodiments, a yeast cell comprises a polynucleotide encoding a de-epitoped HMW glutenin. In some embodiments, a mammalian cell comprises a polynucleotide encoding a de-epitoped HMW glutenin.

In some embodiments, a bacterial cell comprises an expression vector comprising a nucleotide encoding a de-epitoped HMW glutenin. In some embodiments, a bacterial cell comprises an expression vector comprising a polynucleotide encoding a de-epitoped HMW glutenin. In some embodiments, a yeast cell comprises an expression vector comprising a polynucleotide encoding a de-epitoped HMW glutenin. In some embodiments, a mammalian cell comprises an expression vector comprising a polynucleotide encoding a de-epitoped HMW glutenin.

As used herein, the term “vector” refers to discrete elements that are used to introduce heterologous nucleic acids into cells for either expression or replication thereof. An expression vector includes vectors capable of expressing nucleic acids that are operatively linked with regulatory sequences, such as promoter regions, that are capable of affecting expression of such nucleic acids. Thus, an expression vector may refer to a DNA or RNA construct, such as a plasmid, a phage, recombinant virus or other vector that, upon introduction into an appropriate host cell, results in expression of the nucleic acids. Appropriate expression vectors are well known to those of skill in the art and include those that are replicable in prokaryotic cells and/or eukaryotic cells, and those that remain episomal or those which integrate into the host cell genome.

Disclosed herein, in some embodiments is an expression vector comprising the nucleic acid construct encoding any of the HMW glutenin modified proteins disclosed herein.

The term “recombinant host cell” (or simply “host cell”) as used herein refers to a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

Commonly used expression systems for heterologous protein production include bacterial cells (e.g. E. coli), fungal cells (e.g. S. cerevisiae cells), plant cells (e.g. wheat, tobacco, maize), insect cells (lepidopteran cells), and mammalian cells (for example but not limited to Chinese Hamster Ovary cells).

Expressing the exogenous polynucleotide of the present invention within a host cell for example but not limited to a plant cell, can be effected by transforming one or more cells of the host with the exogenous polynucleotide.

Disclosed herein, in some embodiments is a host cell comprising an expression vector carrying the nucleic acid construct encoding any of the HMW glutenin modified proteins disclosed herein. In one embodiment, the cell or host cell is a prokaryotic cell or a eukaryotic cell. In one embodiment, the eukaryotic cell is a yeast cell, an algae cell, a plant cell, or a mammalian cell. In some embodiments, a plant cell comprises a wheat cell, a maize cell, or a tobacco cell.

Various methods can be used to introduce the expression vector of some embodiments of the invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

Preferably, the transformation is effected by introducing to the host cell a nucleic acid construct which includes the exogenous polynucleotide encoding a de-epitoped HMW glutenin and at least one promoter capable of directing transcription of the exogenous polynucleotide in the host cell. Further details of suitable transformation approaches are provided herein below.

As used herein, the term “promoter” refers to a region of DNA which lies upstream of the transcriptional initiation site of a gene to which RNA polymerase binds to initiate transcription of RNA. The promoter controls where (e.g., which portion of a plant, which organ within an animal, etc.) and/or when (e.g., which stage or condition in the lifetime of an organism) the gene is expressed.

Any suitable promoter sequence can be used by the nucleic acid construct encoding the de-epitoped HMW glutenin. In some embodiments, the promoter is a constitutive promoter, a tissue-specific promoter or a plant-specific promoter, for example but not limited to a wheat promoter).

Suitable constitutive promoters include, for example, CaMV 35S promoter (Odell et al., Nature 313:810-812, 1985); maize Ubi 1 (Christensen et al., Plant Sol. Biol. 18:675-689, 1992); rice actin (McElroy et al., Plant Cell 2:163-171, 1990); rice glutelin (Qu, Le Qing et al. J Exp Bot 59:9, 2417-2424, 2008); pEMU (Last et al., Theor. Appl. Genet. 81:581-588, 1991); and Synthetic Super MAS (Ni et al., The Plant Journal 7: 661-76, 1995). Other constitutive promoters include those in U.S. Pat. Nos. 5,659,026, 5,608,149; 5.608,144; 5,604,121; 5.569,597: 5.466,785; 5,399,680; 5,268,463; and 5,608,142.

According to some embodiments, the promoter is a maize promoter (zein) as described in Joshi, J. Beslin, et al. Physiology and Molecular Biology of Plants 21: 35-42, 2015.

Suitable tissue-specific promoters include, but are not limited to, leaf-specific promoters such as described, for example, by Yamamoto et al., Plant J. 12:255-265, 1997; Kwon et al., Plant Physiol. 105:357-67, 1994; Yamamoto et al., Plant Cell Physiol. 35:773-778, 1994; Gotor et al., Plant J. 3:509-18, 1993; Orozco et al., Plant Mol. Biol. 23:1129-1138, 1993; and Matsuoka et al., Proc. Natl. Acad. Sci. USA 90:9586-9590, 1993.

Suitable wheat specific promoters include, but are not limited to those described in Smirnova, O. G. and Kochetov, A. V. Russ J Genet Appl Res (2012) 2: 434. www(dot)doi(dot)org/10(dot)1134/S2079059712060123.

The nucleic acid construct encoding the de-epitoped HMW glutenin, may in some embodiments, further include an appropriate selectable marker and/or an origin of replication. In some embodiments, the nucleic acid construct utilized is a shuttle vector, which can propagate both in E. coli (wherein the construct comprises an appropriate selectable marker and origin of replication) and be compatible for propagation in cells. The construct comprising a polynucleotide encoding a de-epitoped HMW glutenin can be, for example, a plasmid, a bacmid, a phagemid, a cosmid, a phage, a virus or an artificial chromosome.

The nucleic acid construct disclosed herein can be utilized to stably or transiently transform plant cells. In stable transformation, the exogenous polynucleotide disclosed herein is integrated into the plant genome and as such it represents a stable and inherited trait. In transient transformation, the exogenous polynucleotide is expressed by the cell transformed but it is not integrated into the genome and as such it represents a transient trait.

There are various methods of introducing foreign genes into both monocotyledonous and dicotyledonous plants (Potrykus, I., Annu. Rev. Plant. Physiol., Plant. Mol. Biol. (1991) 42:205-225; Shimamoto et al., Nature (1989) 338:274-276).

The principle methods of causing stable integration of exogenous DNA into plant genomic DNA include two main approaches: (i)Agrobacterium-mediated gene transfer: Klee et al. (1987) Annu. Rev. Plant Physiol. 38:467-486; Klee and Rogers in Cell Culture and Somatic Cell Genetics of Plants, Vol. 6, Molecular Biology of Plant Nuclear Genes, eds. Schell, J., and Vasil, L. K., Academic Publishers, San Diego, Calif (1989) p. 2-25; Gatenby, in Plant Biotechnology, eds. Kung, S. and Arntzen, C. J., Butterworth Publishers, Boston, Mass. (1989) p. 93-112.

(ii) Direct DNA uptake: Paszkowski et al., in Cell Culture and Somatic Cell Genetics of Plants, Vol. 6, Molecular Biology of Plant Nuclear Genes eds. Schell, J., and Vasil, L. K., Academic Publishers, San Diego, Calif (1989) p. 52-68; including methods for direct uptake of DNA into protoplasts, Toriyama, K. et al. (1988) Bio/Technology 6:1072-1074. DNA uptake induced by brief electric shock of plant cells: Zhang et al. Plant Cell Rep. (1988) 7:379-384. Fromm et al. Nature (1986) 319:791-793. DNA injection into plant cells or tissues by particle bombardment, Klein et al. Bio/Technology (1988) 6:559-563; McCabe et al. Bio/Technology (1988) 6:923-926; Sanford, Physiol. Plant. (1990) 79:206-209; by the use of micropipette systems: Neuhaus et al., Theor. Appl. Genet. (1987) 75:30-36; Neuhaus and Spangenberg, Physiol. Plant. (1990) 79:213-217; glass fibers or silicon carbide whisker transformation of cell cultures, embryos or callus tissue, U.S. Pat. No. 5,464,765 or by the direct incubation of DNA with germinating pollen, DeWet et al. in Experimental Manipulation of Ovule Tissue, eds. Chapman, G. P. and Mantell, S. H. and Daniels, W. Longman, London, (1985) p. 197-209; and Ohta, Proc. Natl. Acad. Sci. USA (1986) 83:715-719.

The Agrobacterium system includes the use of plasmid vectors that contain defined DNA segments that integrate into the plant genomic DNA. Methods of inoculation of the plant tissue vary depending upon the plant species and the Agrobacterium delivery system. A widely used approach is the leaf disc procedure which can be performed with any tissue explant that provides a good source for initiation of whole plant differentiation. Horsch et al. in Plant Molecular Biology Manual A5, Kluwer Academic Publishers, Dordrecht (1988) p. 1-9. A supplementary approach employs the Agrobacterium delivery system in combination with vacuum infiltration. The Agrobacterium system is especially viable in the creation of transgenic dicotyledonous plants.

There are various methods of direct DNA transfer into plant cells. In electroporation, the protoplasts are briefly exposed to a strong electric field. In microinjection, the DNA is mechanically injected directly into the cells using very small micropipettes. In microparticle bombardment, the DNA is adsorbed on microprojectiles such as magnesium sulfate crystals or tungsten particles, and the microprojectiles are physically accelerated into cells or plant tissues.

Following stable transformation plant propagation is exercised. The most common method of plant propagation is by seed. Regeneration by seed propagation, however, has the deficiency that due to heterozygosity there is a lack of uniformity in the crop, since seeds are produced by plants according to the genetic variances governed by Mendelian rules. Basically, each seed is genetically different and each will grow with its own specific traits. Therefore, it is preferred that the transformed plant be produced such that the regenerated plant has the identical traits and characteristics of the parent transgenic plant. Therefore, it is preferred that the transformed plant be regenerated by micropropagation which provides a rapid, consistent reproduction of the transformed plants.

Micropropagation is a process of growing new generation plants from a single piece of tissue that has been excised from a selected parent plant or cultivar. This process permits the mass reproduction of plants having the preferred tissue expressing the fusion protein. The new generation plants which are produced are genetically identical to, and have all of the characteristics of, the original plant. Micropropagation allows mass production of quality plant material in a short period of time and offers a rapid multiplication of selected cultivars in the preservation of the characteristics of the original transgenic or transformed plant. The advantages of cloning plants are the speed of plant multiplication and the quality and uniformity of plants produced.

Micropropagation is a multi-stage procedure that requires alteration of culture medium or growth conditions between stages. Thus, the micropropagation process involves four basic stages: Stage one, initial tissue culturing; stage two, tissue culture multiplication; stage three, differentiation and plant formation; and stage four, greenhouse culturing and hardening. During stage one, initial tissue culturing, the tissue culture is established and certified contaminant-free. During stage two, the initial tissue culture is multiplied until a sufficient number of tissue samples are produced to meet production goals. During stage three, the tissue samples grown in stage two are divided and grown into individual plantlets. At stage four, the transformed plantlets are transferred to a greenhouse for hardening where the plants' tolerance to light is gradually increased so that it can be grown in the natural environment.

Although stable transformation is presently preferred, transient transformation of leaf cells, meristematic cells or the whole plant is also envisaged by the present invention.

Transient transformation can be affected by any of the direct DNA transfer methods described above or by viral infection using modified plant viruses.

Viruses that have been shown to be useful for the transformation of plant hosts include CaMV, TMV and BV. Transformation of plants using plant viruses is described in U.S. Pat. No. 4,855,237 (BGV), EP-A 67,553 (TMV), Japanese Published Application No. 63-14693 (TMV), EPA 194,809 (BV), EPA 278,667 (BV); and Gluzman, Y. et al., Communications in Molecular Biology: Viral Vectors, Cold Spring Harbor Laboratory, New York, pp. 172-189 (1988). Pseudovirus particles for use in expressing foreign DNA in many hosts, including plants, is described in WO 87/06261.

Preferably, the virus disclosed herein is avirulent and thus is incapable of causing severe symptoms such as reduced growth rate, mosaic, ring spots, leaf roll, yellowing, streaking, pox formation, tumor formation and pitting. A suitable avirulent virus may be a naturally occurring avirulent virus or an artificially attenuated virus. Virus attenuation may be effected by using methods well known in the art including, but not limited to, sub-lethal heating, chemical treatment or by directed mutagenesis techniques such as described, for example, by Kurihara and Watanabe (Molecular Plant Pathology 4:259-269, 2003), Gal-on et al. (1992), Atreya et al. (1992) and Huet et al. (1994).

Suitable virus strains can be obtained from available sources such as, for example, the American Type culture Collection (ATCC) or by isolation from infected plants. Isolation of viruses from infected plant tissues can be effected by techniques well known in the art such as described, for example by Foster and Tatlor, Eds. “Plant Virology Protocols: From Virus Isolation to Transgenic Resistance (Methods in Molecular Biology (Humana Pr), Vol 81)”, Humana Press, 1998. Briefly, tissues of an infected plant believed to contain a high concentration of a suitable virus, preferably young leaves and flower petals, are ground in a buffer solution (e.g., phosphate buffer solution) to produce a virus infected sap which can be used in subsequent inoculations. Construction of plant RNA viruses for the introduction and expression of non-viral exogenous polynucleotide sequences in plants is demonstrated by the above references as well as by Dawson, W. O. et al., Virology (1989) 172:285-292; Takamatsu et al. EMBO J. (1987) 6:307-311; French et al. Science (1986) 231:1294-1297; and Takamatsu et al. FEBS Letters (1990) 269:73-76.

When the virus is a DNA virus, suitable modifications can be made to the virus itself. Alternatively, the virus can first be cloned into a bacterial plasmid for ease of constructing the desired viral vector with the foreign DNA. The virus can then be excised from the plasmid. If the virus is a DNA virus, a bacterial origin of replication can be attached to the viral DNA, which is then replicated by the bacteria. Transcription and translation of this DNA will produce the coat protein which will encapsidate the viral DNA. If the virus is an RNA virus, the virus is generally cloned as a cDNA and inserted into a plasmid. The plasmid is then used to make all of the constructions. The RNA virus is then produced by transcribing the viral sequence of the plasmid and translation of the viral genes to produce the coat protein(s) which encapsidate the viral RNA.

Construction of plant RNA viruses for the introduction and expression in plants of non-viral exogenous polynucleotide sequences such as those included in the construct disclosed herein is demonstrated by the above references as well as in U.S. Pat. No. 5,316,931.

Techniques for inoculation of viruses to plants may be found in Foster and Taylor, eds. “Plant Virology Protocols: From Virus Isolation to Transgenic Resistance (Methods in Molecular Biology (Humana Pr), Vol 81)”, Humana Press, 1998; Maramorosh and Koprowski, eds. “Methods in Virology” 7 vols, Academic Press, New York 1967-1984; Hill, S. A. “Methods in Plant Virology”, Blackwell, Oxford, 1984; Walkey, D. G. A. “Applied Plant Virology”, Wiley, New York, 1985; and Kado and Agrawa, eds. “Principles and Techniques in Plant Virology”, Van Nostrand-Reinhold, New York.

Mature plants generated from the transformed cells may then be cultivated under conditions suitable for expressing the exogenous polynucleotide within the mature plant.

In one embodiment, the plant host cell in which the expression construct is transfected does not naturally express gluten polypeptides (i.e. derived from a non-gluten plant). Thus, in one embodiment, the host cell is selected from the group consisting of amaranth, buckwheat, rice (brown, white, wild), corn millet, quinoa, sorghum, Montina, Job's tears and teff

In another embodiment, the plant host cell in which the expression construct is transfected expresses wild-type gluten polypeptides. Such host cells include but are not limited to wheat varieties such as spelt, kamut, farro and durum, bulger, semolina, barley, rye, triticale, Triticum (wheat cultivars—fielder, spelling, bobwhite, Cheyenne, chinse spring and Mjolnir) and oats. It will be appreciated that in host cells that naturally express gluten polypeptides, the present inventors further contemplate down-regulating expression of the wild-type gluten polypeptides. Methods of down-regulating expression of wild-type gluten polypeptides are known in the art and include for example the use of RNA silencing agent and DNA editing agents. Examples of RNA silencing agents include, but are not limited to siRNA, miRNA, antisense molecules, DNAzyme, RNAzyme. One method of downregulating expression of gluten polypeptides has been described in Sanchez-Le6n, Susana et al. “Low-gluten, Nontransgenic Wheat Engineered with CRISPR/Cas9.” Plant Biotechnology Journal 16.4 (2018): 902-910. PMC, the contents of which are incorporated herein by reference.

### Methods of Producing a De-Epitoped High Molecular Weight Glutenin

Disclosed herein is a method of producing a de-epitoped HMW glutenin comprising (a) culturing cells that comprise an expression vector comprising a polynucleotide encoding any of the de-epitoped HMW glutenin modified proteins disclosed herein, wherein the culturing is under conditions allowing for expression of the de-epitoped HMW glutenin in said cells; and (b) expressing the de-epitoped HMW glutenin. Disclosed herein is a method of producing a de-epitoped HMW glutenin comprising (a) culturing cells that comprise an expression vector comprising a polynucleotide encoding any of the de-epitoped HMW glutenin modified proteins disclosed herein, wherein the culturing is under conditions allowing for expression of the de-epitoped HMW glutenin in said cells; (b) expressing the de-epitoped HMW glutenin; and (c) collecting the expressed de-epitoped HMW glutenin. In some embodiments, the cell comprises a plant cell. In some embodiments, the cell comprises a wheat cell.

In some embodiments, a method of producing a de-epitoped HMW glutenin produces a de-epitoped HMW glutenin comprising substitution mutations, or deletion mutations, or a combination thereof in the repeating allergic unit of the HMW glutenin.

In some embodiments, a method of producing a de-epitoped HMW glutenin produces a de-epitoped HMW glutenin comprising substitution mutations in a repeating antigenic unit of the de-epitoped HMW glutenin. In some embodiments, a method of producing a de-epitoped HMW glutenin produces a de-epitoped HMW glutenin comprising deletion mutations of at least one repeating antigenic unit of the de-epitoped HMW glutenin.

In some embodiments, a method of producing a de-epitoped HMW glutenin produces a de-epitoped HMW glutenin comprising substitution mutations in at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 repeating antigenic units of the de-epitoped HMW glutenin. In some embodiments, a method of producing a de-epitoped HMW glutenin produces a de-epitoped HMW glutenin comprising deletion mutations of at least at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 repeating antigenic units of the de-epitoped HMW glutenin.

In some embodiments, a method of producing a de-epitoped HMW glutenin produces a de-epitoped HMW glutenin comprising substitution mutations in at least 50%, 60%, 70%, 80%, 90% or all of the antigenic units of the de-epitoped HMW glutenin. In some embodiments, a method of producing a de-epitoped HMW glutenin produces a de-epitoped HMW glutenin comprising deletion mutations of at least at least 50%, 60%, 70%, 80%, 90%; or all of the antigenic units may be replaced of the de-epitoped HMW glutenin.

De-epitoped HMW glutenin modified proteins have been described in detail in the section above. In some embodiments, a method of producing a de-epitoped HMW glutenin produces any of the de-epitoped HMW glutenin modified proteins disclosed herein.

In some embodiments, a method of producing a de-epitoped HMW glutenin produces a de-epitoped HMW glutenin comprising a mutation in at least position 1 and position 9 of a repeating antigenic unit of said HMW glutenin. In some embodiments, a method of producing a de-epitoped HMW glutenin produces a de-epitoped HMW glutenin comprising a mutation in position 1 and position 9 of a repeating antigenic unit of said HMW glutenin, and further comprising at least two additional mutations in any of positions 3, 4, or 7 of said repeating antigenic unit. In some embodiments, a method of producing a de-epitoped HMW glutenin produces a de-epitoped HMW glutenin comprising a mutation in position 1 and position 9 of a repeating antigenic unit of said HMW glutenin, and further comprising at least two additional mutations in any of positions 2, 3, or 8 of said repeating antigenic unit.

In some embodiments, a method of producing a de-epitoped HMW glutenin produces a de-epitoped HMW glutenin comprising the amino acid sequence set forth in any of SEQ ID NO: 38, 40-42 or 108-125. In some embodiments, a method of producing a de-epitoped HMW glutenin produces a de-epitoped HMW glutenin comprising the amino acid sequence set forth in any of SEQ ID NO: 109-112, 114-116, or 118-119. In some embodiments, a method of producing a de-epitoped HMW glutenin produces a de-epitoped HMW glutenin comprising the amino acid sequence set forth in any of SEQ ID NO: 126-142, 148-151, or 155-168, 170-172. In some embodiments, a method of producing a de-epitoped HMW glutenin produces a de-epitoped HMW glutenin comprising the amino acid sequence set forth in any of SEQ ID NO: 126-134, 137-138, 140-142, 148-151,155-166. In some embodiments, a method of producing a de-epitoped HMW glutenin produces a de-epitoped HMW glutenin comprising an amino acid sequence set forth in any of SEQ ID NO: 102, 103, 104, 105, 106, 107, 167, 168, 170, 171, or 172. In some embodiments, a method of producing a de-epitoped HMW glutenin produces a de-epitoped HMW glutenin comprising an amino acid sequence set forth in any of SEQ ID NO: 38, 40-42, 102-142, 148-151, or 155-172. In some embodiments, a method of producing a de-epitoped HMW glutenin produces a de-epitoped HMW glutenin comprising the amino acid sequence set forth in any of SEQ ID NO: 102-107, 109-112, 114-116, 118-119, 126-134, 137-138, 140-142, 148-151,155-168, or 170-172.

For generation of recombinant de-epitoped HMW glutenin modified proteins, in some embodiments, production of the modified proteins comprises expression of nucleotide constructs that include sequences engineered to enhance stability, production, purification, or yield of the expressed proteins. For example, the expression of a fusion protein or a cleavable fusion protein comprising a modified glutenin protein of some embodiments of the invention and a heterologous protein can be engineered. Such a fusion protein can be designed so that the fusion protein can be readily isolated by affinity chromatography; e.g., by immobilization on a column specific for the heterologous protein. Where a cleavage site is engineered between a modified glutenin protein and the heterologous protein, a modified glutenin protein can be released from the chromatographic column by treatment with an appropriate enzyme or agent that disrupts the cleavage site [e.g., see Booth et al. (1988) Immunol. Lett. 19:65-70; and Gardella et al., (1990) J. Biol. Chem. 265:15854-15859].

Recovery of the recombinant polypeptide is effected following an appropriate time in culture. The phrase “recovering the recombinant polypeptide” encompasses collecting the whole culture medium, for example a fermentation medium containing the modified HMW glutenin polypeptide. In some embodiments, collecting comprises additional steps of separation or purification. In some embodiments, collecting does not comprises additional steps of separation or purification.

Notwithstanding the above, modified HMW glutenin polypeptides of some embodiments disclosed herein may be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential solubilization.

### Method of De-Epitoping HMW Glutenin

In some embodiments, disclosed herein is a method of de-epitoping HMW glutenin, comprising mutating the amino acid residue at position 1 and 9 of at least one repeating antigenic unit of the glutenin, said non-mutated repeating antigenic unit having an amino acid sequence selected from the group consisting of SEQ ID NOs: 1-33, 35, or 71-99, thereby generating a de-epitoped high molecular weight glutenin. In some embodiments, the method of de-epitoping a HMW glutenin, further comprises mutating at least two amino acids at positions 3, 4, or 7 of said repeating antigenic unit. In some embodiments, the method of de-epitoping a HMW glutenin, further comprises mutating at least two amino acids at positions 2, 3, and 8, of said repeating antigenic unit. In some embodiments, the method of de-epitoping a HMW glutenin, further comprises mutating at least two amino acids at positions 3, 5, and 8, of said repeating antigenic unit.

In some embodiments of a method of de-epitoping a HMW glutenin, the mutations comprise substitution mutations. In some embodiments, of a method of de-epitoping a HMW glutenin, the mutations comprise deletion mutations within at least one antigenic unit. In some embodiments, of a method of de-epitoping a HMW glutenin, the mutations comprise deletion mutations of at least one antigenic unit.

In some embodiments, the mutations to said repeating antigenic unit do not disrupt the function of the HMW glutenin polypeptide (for example but not limited to not disrupting the function of the modified HMW glutenin polypeptide relative to the function of the corresponding un-modified HMW glutenin polypeptide).

In some embodiments, the mutations to a repeating antigenic unit does not disrupt at least one of the following characteristics: (1) the dough strengthening ability of the HMW glutenin protein; (2) the dough elasticity promoting ability of the polypeptide; (3) the dough rising promoting ability of the polypeptide; (4) the growth of a plant comprising the modified HMW glutenin, for example but not limited to wheat, wherein the production of seeds, number of seeds, or size of seeds is not disrupted; (5) native protein-protein interactions of the de-epitoped HMW glutenin polypeptide (e.g., the modified HMW glutenin polypeptide retains the ability to form substantially the same protein-protein interactions as the corresponding un-mutated polypeptide); (6) the three-dimensional structure of the HMW glutenin polypeptide (e.g., the modified de-epitoped HMW glutenin polypeptide retains substantially the same three-dimensional structure as the corresponding un-modified HMW glutenin polypeptide); (7) the folding of the HMW glutenin polypeptide (e.g., the modified de-epitoped HMW glutenin polypeptide retains substantially the same protein folding as the corresponding un-modified HMW glutenin polypeptide); (8) the translation of the HMW glutenin polypeptide (e.g., the modified de-epitoped HMW glutenin polypeptide is translated with the same timing, at the same rate, to the same levels, etc. as the corresponding un-modified HMW glutenin polypeptide); (9) the normal cellular localization of the HMW glutenin polypeptide (e.g., the modified de-epitoped HMW glutenin polypeptide retains substantially the same cellular localization as the corresponding un-modified HMW glutenin polypeptide); (10) any post-translational modifications on the HMW glutenin polypeptide (e.g., the modified de-epitoped HMW glutenin polypeptide retains substantially the same post-translational modification profile as the corresponding un-modified HMW glutenin polypeptide); and (11) the allergenicity of a wheat HMW glutenin polypeptide (e.g., the modified de-epitoped HMW glutenin retains substantially the same IgE antibody binding affinity as the corresponding un-modified HMW glutenin polypeptide). In some embodiments, the mutations to a repeating antigenic unit disrupts the allergenicity of a HMW glutenin polypeptide (e.g., the modified de-epitoped HMW glutenin has reduced IgE antibody binding affinity compared with the binding affinity of a wheat extract).

In some embodiments, the mutations to a repeating antigenic unit for de-epitoping a HMW glutenin does not affect at least two, three, four, five or more of the parameters described herein above. In some embodiments, the mutations to a repeating antigenic unit for de-epitoping a HMW glutenin does not affect any of the parameters described herein above.

Methods for checking the protein structure/fold/biochemical-biophysical properties of the de-epitoped glutenin disclosed herein include hydrodynamic studies (see for example Field, J. M., Tatham, A. S. & Shewry, P. R. 1987. Biochem. J. 247, 215-221; Castellia, F. et al., 2000. Thermochimica Acta 346, 153-160); NMR spectroscopy (see for example Bekkers, A. C., et al. 1996, In Glutenin 96 Proc. 6th Int. Wheat Glutenin Workshop, Sydney, September 1996 pp. 190-194. North Melbourne, Australia: Royal Australian Chemical Institute; Eliezer, D., Biophysical characterization of intrinsically disordered proteins. Curr Opin Struct Biol. 2009; 19(1):23-30); Circular dichroism measurements (see for example Tatham, A. S., Shewry, P. R., 1985. J. Cereal Sci. 3, 104-113); Heterologous expression analysis (see for example Tatham, A. S., Shewry, P. R., 1985. J. Cereal Sci. 3, 104-113); Static and dynamic light scattering measurements (see for example Herrera, M.; Dodero, V. In Proceedings of the F. Bioact. Process. Qual. & Nutr., 10-12 Apr. 2013; Sciforum Electronic Conferences Series; T. A. Egorov, FEBS Letters, Volume 434, Issues 1-2, 1998, Pages 215-217); Small-angle X-ray scattering (see for example Neil H. Thomson Biochimica et Biophysica Acta (BBA)—Protein Structure and Molecular Enzymology, Volume 1430, Issue 2, 1999, Pages 359-366; Eliezer, D., Curr Opin Struct Biol. 2009; 19(1):23-30); very-small-angle Neutron scattering (see for example Mohsen Daheshet al., The Journal of Physical Chemistry B 2014 118 (38), 11065-11076. DOI: 10.1021/jp5047134; Gibbs, B. E. & Showalter, S. A. 2015, Biochemistry 54, 1314-1326; fluorescence correlation spectroscopy (FCS) (see for example Eliezer, D., Curr Opin Struct Biol. 2009; 19(1):23-3); and Single-Molecule FRET (smFRET) (see for example Gibbs, B. E. & Showalter, S. A. 2015, Biochemistry 54, 1314-1326). The contents of all the above-described references are incorporated herein by reference.

In some embodiments a method of de-epitoped HMW glutenin disclosed herein, produces a modified HMW glutenin that binds with a poorer affinity to celiac related MHCII proteins (e.g. HLA-DQ8; GenBank Accession numbers: DQ8 alpha chain—MK501646.1 (SEQ ID NO: 34), beta chain—OU241996.1(SEQ ID NO: 139),) or to T-cells derived from a celiac patient than a corresponding non-modified HMW glutenin binds to MHCII proteins or T cells derived from the same celiac patient. Furthermore, the de-epitoped HMW glutenin may bind with a poorer affinity to DQ7.5 MHCII proteins than a corresponding non-modified HMW glutenin binds to DQ7.5 MHCII proteins.

In some embodiments a method of de-epitoped HMW glutenin, produces a modified HMW glutenin that does not comprise an epitope that bind to MHC class DQ2 or DQ8 proteins with an IC50 less than 30 μM. In some embodiments, a de-epitoped high molecular weight glutenin does not comprise an antigenic unit that bind to MHC class DQ2 or DQ8 proteins with an IC50 less than 30 μM. In some embodiments, a de-epitoped high molecular weight glutenin does not comprise an antigenic unit comprising a 15-mer peptide that bind to MHC class DQ2 or DQ8 proteins with an IC50 less than 30 μM. In some embodiments, a de-epitoped high molecular weight glutenin does not comprise an antigenic unit comprising a 14-mer peptide that bind to MHC class DQ2 or DQ8 proteins with an IC50 less than 30 μM. In some embodiments, a de-epitoped high molecular weight glutenin does not comprise an antigenic unit comprising a 13, 12, 11, 10, or 9-mer, peptide that bind to MHC class DQ2 or DQ8 proteins with an IC50 less than 30 μM. In some embodiments, peptides derived from a de-epitoped high molecular weight glutenin do not bind to MHC class DQ2 or DQ8 proteins with an IC50 less than 30 μM. In some embodiments, a de-epitoped high molecular weight glutenin does not comprise an antigenic unit that binds to MHC class DQ2 or DQ8 proteins with an IC50 less than 30 μM.

In some embodiments a method of de-epitoped HMW glutenin, produces a modified HMW glutenin that does not comprise an epitope that bind to MHC class DQ2 or DQ8 proteins with an IC50 less than 25 μM. In some embodiments, a de-epitoped high molecular weight glutenin does not comprise an antigenic unit that bind to MHC class DQ2 or DQ8 proteins with an IC50 less than 25 μM. In some embodiments, a de-epitoped high molecular weight glutenin does not comprise an antigenic unit comprising a 15-mer peptide that bind to MHC class DQ2 or DQ8 proteins with an IC50 less than 25 μM. In some embodiments, a de-epitoped high molecular weight glutenin does not comprise an antigenic unit comprising a 14-mer peptide that bind to MHC class DQ2 or DQ8 proteins with an IC50 less than 25 μM. In some embodiments, a de-epitoped high molecular weight glutenin does not comprise an antigenic unit comprising a 13, 12, 11, 10, or 9-mer, peptide that bind to MHC class DQ2 or DQ8 proteins with an IC50 less than 25 μM. In some embodiments, peptides derived from a de-epitoped high molecular weight glutenin do not bind to MHC class DQ2 or DQ8 proteins with an IC50 less than 25 μM. In some embodiments, a de-epitoped high molecular weight glutenin does not comprise an antigenic unit that binds to MHC class DQ2 or DQ8 proteins with an IC50 less than 25 μM.

In some embodiments a method of de-epitoped HMW glutenin, produces a modified HMW glutenin that does not comprise an epitope that bind to MHC class DQ2 or DQ8 proteins with an IC50 less than 20 μM. In some embodiments, a de-epitoped high molecular weight glutenin does not comprise an antigenic unit that bind to MHC class DQ2 or DQ8 proteins with an IC50 less than 20 μM. In some embodiments, a de-epitoped high molecular weight glutenin does not comprise an antigenic unit comprising a 15-mer peptide that bind to MHC class DQ2 or DQ8 proteins with an IC50 less than 20 μM. In some embodiments, a de-epitoped high molecular weight glutenin does not comprise an antigenic unit comprising a 14-mer peptide that bind to MHC class DQ2 or DQ8 proteins with an IC50 less than 20 μM. In some embodiments, a de-epitoped high molecular weight glutenin does not comprise an antigenic unit comprising a 13, 12, 11, 10, or 9-mer, peptide that bind to MHC class DQ2 or DQ8 proteins with an IC50 less than 20 μM. In some embodiments, peptides derived from a de-epitoped high molecular weight glutenin do not bind to MHC class DQ2 or DQ8 proteins with an IC50 less than 20 μM. In some embodiments, a de-epitoped high molecular weight glutenin does not comprise an antigenic unit that binds to MHC class DQ2 or DQ8 proteins with an IC50 less than 20 μM.

Thus, the affinity value, measured in units of concentration, is at least 10%, 20%, 30 00 40%, 50%, 60%, 70% 80%, 90% or 100% higher for the de-epitoped HMW glutenin protein binding to celiac related MHCII proteins (e.g. HLA-DQ2 or HLA-DQ8) or to T-cells derived from a celiac patient than a corresponding non-modified HMW glutenin binds to T cells derived from the same celiac patient.

A skilled artisan would appreciate that a higher affinity value indicates weaker binding, in that a higher concentration is needed to reach the same number of bound receptors or in the case of IC50, to reduce by half the levels of the bound control peptide. In some embodiments, a de-epitoped HMW glutenin disclosed herein, has reduced binding affinity to celiac related MHCII proteins or to T-cells derived from a celiac patient than a non-de-epitoped HMW glutenin. In some embodiments, peptides derived from a de-epitoped HMW glutenin disclosed herein, have reduced binding affinity to T-cells derived from a celiac patient than peptides derived from a non-de-epitoped HMW glutenin. In some embodiments, repeating antigenic units derived from a de-epitoped HMW glutenin disclosed herein, have reduced binding affinity to celiac related MHCII proteins or to T-cells derived from a celiac patient than repeating antigenic units derived from a non-de-epitoped HMW glutenin.

In some embodiments, if HMW proteins are digested and then binding to HLA-DQ2.5/8 is tested, there are peptides that bind these MHCII proteins, but do not activate T-cells, due to non-binding to T-cells at the required affinity. In some embodiments, an antigenic unit or a de-epitoped HMW glutenin showing binding to HLA-DQ2.5/8 of greater than 30 uM indicates said antigenic unit or de-epitoped HMW glutenin has a low loading capacity to MHC II, and consequently cannot be effectively recognized by T-cells, thus leading to T-cell non-activation

In one embodiment, the binding of a modified de-epitoped HMW glutenin to celiac related MHCII proteins (e.g. HLA-DQ2 or HLA-DQ8) or to T cells, is abrogated. Methods of measuring the binding of HMW glutenin antigenic unit peptides/HMW glutenin polypeptides to Celiac related MHCII proteins (e.g. HLA-DQ2 or HLA-DQ8) or to T cells are known in the art and include for example: 1) detection of peptide/MHCII complexes using a combination of gel-filtration and competitive binding to a well-defined radio-labeled reference peptide (Sidney, John, et al. “Measurement of MHC/peptide interactions by gel filtration or monoclonal antibody capture.” Current protocols in immunology 100.1 (2013): 18-3); 2) Using MHCII tetramers with gluten peptides fusion to detect and quantify binding to gluten-specific CD4+ T cells by flow cytometer (Riki, Melinda, et al. “Tetramer visualization of gut-homing gluten-specific T cells in the peripheral blood of celiac disease patients.” Proceedings of the National Academy of Sciences 104.8 (2007): 2831-2836.); 3) ELISpot or ELISA assay to measure activation of gluten-specific CD4+ T cells by probing secretion of IFN-γ Anderson, R. P., et al. “T cells in peripheral blood after gluten challenge in coeliac disease.” Gut 54.9 (2005): 1217-1223.); 4) Proliferation assays of gluten-specific T cells in the presence of relevant APCs (e.g., HLA DQ8 or HLA DQ2.5 expressing cells) and gluten peptides (Kooy-Winkelaar, Yvonne, et al. “Gluten-specific T cells cross-react between HLA-DQ8 and the HLA-DQ2a/DQ80 transdimer.” The Journal of Immunology 187.10 (2011): 5123-5129.).

According to some embodiments, a method of de-epitoped HMW glutenin disclosed herein, produces a modified HMW glutenin that does not comprise 15 mer peptides present in the repeating antigenic unit that bind to MHC class DQ2 or DQ8 with an IC50 of less than 20 μM, less than 30 μM or even less than 40 μM—see Example 1 herein below. According to some embodiments, a method of de-epitoped HMW glutenin disclosed herein, produces a modified HMW glutenin that does not comprise a repeating motif present in the repeating antigenic unit that bind to MHC class DQ2 or DQ8 with an IC50 of less than 20 μM, less than 30 μM or even less than 40 μM. In some embodiments, a method of de-epitoped HMW glutenin disclosed herein, produces a modified HMW glutenin that does not comprise 9-15-mer peptides present in the repeating antigenic unit that bind to MHC class DQ2 or DQ8 with an IC50 of less than 20 μM, less than 30 μM or even less than 40 μM. In some embodiments, a method of de-epitoped HMW glutenin disclosed herein, produces a modified HMW glutenin that does not comprise repeating antigenic units that bind to MHC class DQ2 or DQ8 with an IC50 of less than 20 μM, less than 30 μM or even less than 40 μM.

In some embodiments of a method of de-epitoped HMW glutenin, the method produces a modified HMW glutenin that activates T-cells derived from a celiac patient to a lesser extent (e.g. by at least 10%, 20%, 30%, 40%, 50%, 60%, 70% 80%, 90% or 100%) than a corresponding non-mutated polypeptide activates T cells derived from the same celiac patient. An exemplary T cell activation assay includes the measurement of IFN-7 using ELISA.

As described throughout, the de-epitoping of a HMW glutenin is carried out in some embodiments, by substituting the first amino acid (i.e. position 1) and the ninth amino acid (i.e. position 9) of at least one repeating antigenic unit of the HMW glutenin. In certain embodiments, de-epitoping of a HMW glutenin is carried out by substituting the first amino acid (i.e. position 1) and the ninth amino acid (i.e. position 9), as well as substituting at least two additional amino acids within of at least one repeating antigenic unit of the HMW glutenin.

The substituting can be by conservative or non-conservative substitutions.

The phrase “non-conservative substitutions” may encompass replacement of the amino acid as present in the parent (WT) HMW glutenin sequence by another naturally or non-naturally occurring amino acid, having different electrochemical and/or steric properties. Thus, the side chain of the substituting amino acid can be significantly larger (or smaller) than the side chain of the native amino acid being substituted and/or can have functional groups with significantly different electronic properties than the amino acid being substituted. Examples of non-conservative substitutions of this type include the substitution of phenylalanine or cycohexylmethyl glycine for alanine, isoleucine for glycine, or NH CH[(CH2)5 COOH] CO for aspartic acid.

It will be appreciated that conservative substitutions may also be contemplated herein. Conservative substitution tables providing functionally similar amino acids are well known in the art. Guidance concerning which amino acid changes are likely to be phenotypically silent can also be found in Bowie et al., 1990, Science 247: 1306 1310. Such conservatively modified variants are in addition to and do not exclude polymorphic variants, interspecies homologs, and alleles. Typical conservative substitutions include but are not limited to: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Thomas E. Proteins: structures and molecular properties. Macmillan, 1993). Amino acids can be substituted based upon properties associated with side chains, for example, amino acids with polar side chains may be substituted, for example, Serine (S), Threonine (T), Tyrosine (Y), Aspartic Acid (D) and Glutamic Acid (E); amino acids based on the electrical charge of a side chain, for example positively or negatively charges amino acids, Arginine (R), Histidine (H), Lysine (K), Aspartic Acid (D), and Glutamic Acid (E); small amino acids Serine (S), threonine (T), alanine (A), glycine (G), and valine (V); aliphatic amino acids methionine (M), valine (V), leucine (L), isoleucine (I) and alanine (A); aromatic amino acids phenylalanine (F), tryptophan (W) and tyrosine (Y); and amino acids that have hydrophobic side chains, for example, Valine (V) Leucine (L), Methionine (M) and Isoleucine (I). As indicated, changes are typically of a minor nature, such as conservative amino acid substitutions that do not significantly affect the folding or activity of the protein.

In some embodiments of a method of de-epitoping a HMW glutenin, mutating comprises substituting the amino acid residues at position 9 of the antigenic unit, wherein the substituting comprises a replacement with a positively charged or small amino acid, or a glutamate. In some embodiments of a method of de-epitoping a HMW glutenin, a positively charged amino acid comprises an arginine, histidine or lysine; or the small amino acid comprises serine or threonine; or the substituting is a combination of wherein more than one antigenic unit is mutated. In other embodiments of a method of de-epitoping a HMW glutenin, the mutating comprises substituting at position 9 of the antigenic unit, wherein there is a replacement with an arginine, histidine, serine, threonine, or a glutamate.

In some embodiments of a method of de-epitoping a HMW glutenin, mutating comprises substituting at position 1 of the antigenic unit, the substituting comprises a replacement with a histidine, a proline, a serine, an alanine, lysine, or a glutamate. In some embodiments of a method of de-epitoping a HMW glutenin, (a) the substituting at position 1 of said antigenic unit is a replacement with histidine, and the substituting at position 9 of the antigenic unit is a replacement with histidine; (b) the substituting at position 1 of the antigenic unit is a replacement with proline, and the substituting at position 9 of the antigenic unit is a replacement with arginine; (c) the substituting at position 1 of the antigenic unit is a replacement with alanine, and the substituting at position 9 of the antigenic unit is a replacement with arginine; (d) the substituting at position 1 of the antigenic unit is a replacement with serine, and the substituting at position 9 of the antigenic unit is a replacement with threonine; (e) the substituting at position 1 of the antigenic unit is a replacement with glutamate, and the substituting at position 9 of the antigenic unit is a replacement with glutamate; or (f) the substituting at position 1 of the antigenic unit is a replacement with proline, and the substituting at position 9 of the antigenic unit is a replacement with threonine; (g) the substituting at position 1 of the antigenic unit is a replacement with glutamate, and the substituting at position 9 of the antigenic unit is a replacement with lysine or arginine; (h) the substituting at position 1 of the antigenic unit is a replacement with glutamate or a lysine, and the substituting at position 9 of the antigenic unit is a replacement with histidine; or (i) any combination of (a)-(h) when more than one repeating antigenic unit is mutated.

In some embodiments of a method of de-epitoping a HMW glutenin, the method further comprises mutating at least two amino acids at positions 3, 4, or 7 of the repeating antigenic unit. In some embodiments of the method, mutating comprises substituting amino acid residues at these positions. In some embodiments of the method, (a) the substituting at position 3 is replacing the amino acid with a polar or positive amino acid; or (b) the substituting at position 4 is replacing the amino acid with a negatively charged amino acid or glycine; or (c) the substituting at position 7 is replacing the amino acid with glycine; or the substituting is any combination of (a)-(c) when more than one repeating antigenic unit is mutated.

In some embodiments of a method of de-epitoping a HMW glutenin, the method further comprises mutating at least two amino acids at positions 2, 3, or 8 of the repeating antigenic unit. In some embodiments of the method, the mutating is substituting, wherein (a) the substituting at position 2 is replacing the amino acid with an aromatic amino acid or a positively charged amino acid; or (b) the substituting at position 3 is replacing the amino acid with a polar or a positively charged amino acid; or (c) the substituting at position 8 is replacing the amino acid with a small or aliphatic amino acid; or the substituting comprises any combination of (a)-(c) when more than one repeating antigenic unit is mutated. In some embodiments of a method of de-epitoping a HMW glutenin, the substituting at position 2 is replacing the amino acid with tryptophan and the substituting at position 3 is replacing the amino acid with histidine.

In some embodiments of a method of de-epitoping a HMW glutenin, the method further comprises mutating at least two amino acids at positions 3, 5 or 8 of the repeating antigenic unit. In some embodiments of the method, the mutating is substituting, wherein (a) the substituting at position 3 is replacing the amino acid with a polar amino acid or a positively charged amino acid; or (b) the substituting at position 5 is replacing the amino acid with a hydrophobic amino acid; or (c) the substituting at position 8 is replacing the amino acid with a small or aliphatic amino acid; or the substituting comprises any combination of (a)-(c) when more than one repeating antigenic unit is mutated. In some embodiments of a method of de-epitoping a HMW glutenin, the substituting at position 3 is replacing the amino acid with arginine and the substituting at position 5 is replacing the amino acid with leucine.

In some embodiments of a method of de-epitoping a HMW glutenin, the de-epitoped HMW glutenin comprises an amino acid sequence set forth in any of SEQ ID NOs: 38, 40-42, SEQ ID NO: 52, SEQ ID NOs: 102-107, SEQ ID NOs: 108-125, SEQ ID NOs: 126-142, SEQ ID NOs: 148-151, and SEQ ID NOs: 155-172. In some embodiments of a method of de-epitoping a HMW glutenin, the de-epitoped HMW glutenin comprises an amino acid sequence set forth in any of SEQ ID NOs: 52, 102-107, 109-112, 114-116, 118-119, 126-134, 137-138, 140-142, 148-151, 155-168, and 170-172. In some embodiments of a method of de-epitoping a HMW glutenin, the de-epitoped HMW glutenin comprises an amino acid sequence set forth in any of SEQ ID NOs: 38, 40-42. In some embodiments of a method of de-epitoping a HMW glutenin, the de-epitoped HMW glutenin comprises an amino acid sequence set forth in any of SEQ ID NOs: 52. In some embodiments of a method of de-epitoping a HMW glutenin, the de-epitoped HMW glutenin comprises an amino acid sequence set forth in any of SEQ ID NOs: 102-107. In some embodiments of a method of de-epitoping a HMW glutenin, the de-epitoped HMW glutenin comprises an amino acid sequence set forth in any of SEQ ID NOs: SEQ ID NOs: 109-112. In some embodiments of a method of de-epitoping a HMW glutenin, the de-epitoped HMW glutenin comprises an amino acid sequence set forth in any of SEQ ID NOs: 114-116. In some embodiments of a method of de-epitoping a HMW glutenin, the de-epitoped HMW glutenin comprises an amino acid sequence set forth in any of SEQ ID NOs: 118-119. In some embodiments of a method of de-epitoping a HMW glutenin, the de-epitoped HMW glutenin comprises an amino acid sequence set forth in any of SEQ ID NOs: 108-125. In some embodiments of a method of de-epitoping a HMW glutenin, the de-epitoped HMW glutenin comprises an amino acid sequence set forth in any of SEQ ID NOs: 126-142. In some embodiments of a method of de-epitoping a HMW glutenin, the de-epitoped HMW glutenin comprises an amino acid sequence set forth in any of SEQ ID NOs: 126-134. In some embodiments of a method of de-epitoping a HMW glutenin, the de-epitoped HMW glutenin comprises an amino acid sequence set forth in any of SEQ ID NOs: 137-138. In some embodiments of a method of de-epitoping a HMW glutenin, the de-epitoped HMW glutenin comprises an amino acid sequence set forth in any of SEQ ID NOs: 140-142. In some embodiments of a method of de-epitoping a HMW glutenin, the de-epitoped HMW glutenin comprises an amino acid sequence set forth in any of SEQ ID NOs: 148-151. In some embodiments of a method of de-epitoping a HMW glutenin, the de-epitoped HMW glutenin comprises an amino acid sequence set forth in any of SEQ ID NOs: 155-172. In some embodiments of a method of de-epitoping a HMW glutenin, the de-epitoped HMW glutenin comprises an amino acid sequence set forth in any of SEQ ID NOs: 155-168 In some embodiments of a method of de-epitoping a HMW glutenin, the de-epitoped HMW glutenin comprises an amino acid sequence set forth in any of SEQ ID NOs: 170-172. In some embodiments of a method of de-epitoping a HMW glutenin, the de-epitoped HMW glutenin comprises an amino acid sequence set forth in any of SEQ ID NOs: 126-134, 137, 138, 148, and 155-162. In some embodiments of a method of de-epitoping a HMW glutenin, the de-epitoped HMW glutenin comprises an amino acid sequence set forth in any of SEQ ID NOs: 109, 111, 115, 116, 118, and 119.

In some embodiments of a method of de-epitoping a HMW glutenin, the de-epitoped HMW glutenin comprises an amino acid sequence set forth in any of SEQ ID NOs: 102-107, 167-168, and 170-172, wherein the repeating antigenic units have been deleted. In some embodiments of a method of de-epitoping a HMW glutenin, the de-epitoped HMW glutenin comprises an amino acid sequence set forth in any of SEQ ID NOs: 109-112, 114-116, 118-119, 126-134, 137-138, 140-142, 148-151, 155-166, wherein the repeating antigenic units comprise substitution mutations.

In some embodiments of a method of de-epitoping a HMW glutenin, the method further comprises analyzing the binding of the de-epitoped high molecular weight glutenin to T-cells derived from a celiac patient; and identifying the de-epitoped high molecular weight glutenin as comprising reduced immunogenicity when the de-epitoped high molecular weight glutenin activates T-cells derived from a celiac patient to a lesser extent than a corresponding non-mutated high molecular weight glutenin activates T cells derived from the celiac patient, as measured using a HLA-DQ-peptide tetramer-based assay or by an interferon-7 ELISA assay; or any combination thereof.

In some embodiments of a method of de-epitoping a HMW glutenin, the mutating is carried out on at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 repeating antigenic units. In some embodiments of a method of de-epitoping a HMW glutenin, the mutating is carried out on more than 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 repeating antigenic units. In some embodiments of a method of de-epitoping a HMW glutenin, the mutating is carried out on 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30, or more than 30 repeating antigenic units.

In some embodiments of a method of de-epitoping a HMW glutenin, the mutating is carried out on 1-30 antigenic units. In some embodiments of a method of de-epitoping a HMW glutenin, the mutating is carried out on 1-20 antigenic units. In some embodiments of a method of de-epitoping a HMW glutenin, the mutating is carried out on 1-5 antigenic units. In some embodiments of a method of de-epitoping a HMW glutenin, the mutating is carried out on 1-10 antigenic units. In some embodiments of a method of de-epitoping a HMW glutenin, the mutating is carried out on 1-15 antigenic units. In some embodiments of a method of de-epitoping a HMW glutenin, the mutating is carried out on 1-20 antigenic units. In some embodiments of a method of de-epitoping a HMW glutenin, the mutating is carried out on 5-20 antigenic units. In some embodiments of a method of de-epitoping a HMW glutenin, the mutating is carried out on 5-10 antigenic units. In some embodiments of a method of de-epitoping a HMW glutenin, the mutating is carried out on 5-15 antigenic units. In some embodiments of a method of de-epitoping a HMW glutenin, the mutating is carried out on 10-20 antigenic units. In some embodiments of a method of de-epitoping a HMW glutenin, the mutating is carried out on 15-20 antigenic units. In some embodiments of a method of de-epitoping a HMW glutenin, the mutating is carried out on more than 20 antigenic units. In some embodiments of a method of de-epitoping a HMW glutenin, the mutating is carried out on 5-30 antigenic units. In some embodiments of a method of de-epitoping a HMW glutenin, the mutating is carried out on 10-30 antigenic units. In some embodiments of a method of de-epitoping a HMW glutenin, the mutating is carried out on 15-30 antigenic units. In some embodiments of a method of de-epitoping a HMW glutenin, the mutating is carried out on 20-30 antigenic units. In some embodiments of a method of de-epitoping a HMW glutenin, the mutating is carried out on 25-30 antigenic units. In some embodiments of a method of de-epitoping a HMW glutenin, the mutating is carried out on 10-25 antigenic units.

In some embodiments of a method of de-epitoping a HMW glutenin, at least 10%, at least 20%, at least 30%, at least 40%, at least 50%, at least 60%, at least 70%, at least 80%, at least 90%, or all of the antigenic units present in the HMW glutenin protein are mutated (i.e. de-epitoped).

In some embodiments of a method of de-epitoping a HMW glutenin, the mutating is carried out on at least five-ten of the repeating antigenic units.

### Food Products

Disclosed herein are uses of the modified de-epitoped HMW glutenin for producing food products particularly beneficial for subjects suffering from a gluten sensitivity, including a glutenin sensitivity, for example a subject suffering from CD or any related irritation of the intestine or bowels. These food products may also benefit a subject suffering from wheat allergies, for example but not limited to wheat dependent exercise induced anaphylaxis (WDEIA), or a subject suffering from non-celiac gluten sensitivity. In some embodiments, the de-epitoped HMW glutenin polypeptides described herein is for the preparation of foods suitable for consumption by a subject having celiac disease. Thus, the de-epitoped HMW glutenin may be used in the preparation of meat products, cheese, and vegetarian alternatives to meat products.

In certain embodiments, the de-epitoped gluten polypeptides can be used in the preparation of edible flour. In some embodiments, disclosed herein is a flour comprising any of the de-epitoped HMW glutenins disclosed herein. In some embodiments, a flour comprising any of the de-epitoped HMW glutenins disclosed herein is derived from a plant lacking WT HMW glutenin. In some embodiments, a flour comprising any of the de-epitoped HMW glutenins disclosed herein is derived from a plant lacking other gluten protein components. In some embodiments, a flour comprising any of the de-epitoped HMW glutenins disclosed herein is derived from a plant lacking at a reduced percent of gluten proteins. Non limiting examples of plants (e.g. grains) from which a flour comprising a de-epitoped HMW glutenin may be derived include but are not limited to amaranth, wheat, buckwheat, rice (brown, white, wild), corn millet, quinoa, sorghum, and teff. In some embodiments, a plant from which a flour comprising a de-epitoped HMW glutenin may be derived comprises a reduced quantity of gluten proteins. In some embodiments, a plant from which a flour comprising a de-epitoped HMW glutenin may be derived comprises about 75% reduction of gluten proteins.

In some embodiments, disclosed herein is a modified wheat expressing a modified de-epitoped HMW glutenin as disclosed herein, wherein at least one antigenic unit is mutated. In some embodiments, disclosed herein is a modified wheat expressing a modified de-epitoped HMW glutenin as disclosed herein, wherein multiple antigenic units are mutated. In some embodiments, disclosed herein is a modified wheat expressing a modified de-epitoped HMW glutenin as disclosed herein, wherein said modified HMW glutenin comprises antigenic unit mutations comprising substitutions mutations or deletions mutations, or a combination thereof, as described herein throughout for de-epitoped HMW glutenins disclosed herein. In some embodiments, disclosed herein is a modified wheat grass expressing a modified de-epitoped HMW glutenin as disclosed herein.

In some embodiments, disclosed herein is a flour comprising any of the de-epitoped HMW glutenins disclosed herein, wherein said flour is derived from a modified wheat grass.

In some embodiments, disclosed herein is a modified corn expressing a modified de-epitoped HMW glutenin as disclosed herein, wherein at least one antigenic unit is mutated. In some embodiments, disclosed herein is a modified corn expressing a modified de-epitoped HMW glutenin as disclosed herein, wherein multiple antigenic units are mutated. In some embodiments, disclosed herein is a modified corn expressing a modified de-epitoped HMW glutenin as disclosed herein, wherein said modified HMW glutenin comprises antigenic unit mutations comprising substitutions mutations or deletions mutations, or a combination thereof, as described herein throughout for de-epitoped HMW glutenins disclosed herein. In some embodiments, disclosed herein is a modified corn plant expressing a modified de-epitoped HMW glutenin as disclosed herein.

In some embodiments, disclosed herein is a flour comprising any of the de-epitoped HMW glutenins disclosed herein, wherein said flour is derived from a modified corn plant.

A skilled artisan would appreciate that the term “flour” may encompass a foodstuff which is a free-flowing powder, typically obtained by milling. Flour is most often used in bakery food products, such as breads, cakes, pastries etc., but also in other food products such as pasta, noodles, breakfast cereals and the like.

Examples of flours include bread flour, all-purpose flour, unbleached flour, self-rising flour, white flour, brown flour, and semolina flour. In some embodiments, there is provided a flour derived from a non-gluten plant, comprising at least one de-epitoped HMW glutenin polypeptide.

In some embodiments, the non-gluten plant is transformed with a polynucleotide encoding a de-epitoped HMW glutenin polypeptide disclosed herein, and a flour is generated therefrom (for example by grinding, mincing, milling etc.).

In some embodiments, a flour is generated from a non-gluten plant (for example by grinding, mincing, milling etc.) and at least one modified de-epitoped HMW glutenin polypeptide disclosed herein is added.

A skilled artisan would appreciate that a non-gluten plant comprises a reduced quantity of gluten proteins. In some embodiments, a non-gluten plant comprises no gluten proteins (level is undetectable). In some embodiments, a non-gluten plant comprises between a 75%-100% reduction of gluten proteins. In some embodiments, a non-gluten plant comprises a 75% reduction of gluten proteins. In some embodiments, a non-gluten plant comprises a 75%, 80%, 85%, 90%, 95%, or 99%, reduction of gluten proteins. In some embodiments, a non-gluten plant comprises at least a 75% reduction of gluten proteins. In some embodiments, a non-gluten plant comprises at least a 75%, 80%, 85%, 90%, 95%, or 99%, reduction of gluten proteins. In some embodiments, a non-gluten plant comprises more than a 75% reduction of gluten proteins. In some embodiments, a non-gluten plant comprises more than a 75%, 80%, 85%, 90%, 95%, or 99%, reduction of gluten proteins.

In some embodiments, wheat is genetically modified to express any of the de-epitoped HMW glutenin disclosed herein. In some embodiments, in wheat genetically modified to express a de-epitoped HMW glutenin, the expression of the corresponding non-mutated HMW glutenin polypeptide is down-regulated compared to a wild-type wheat. In certain embodiments, the genetically modified wheat comprises an RNA silencing agent directed towards the non-mutated polypeptide. In some embodiments, the genetically modified wheat is genetically modified by a DNA editing agent.

In some embodiments, a corn plant is genetically modified to express any of the de-epitoped HMW glutenin disclosed herein. In some embodiments, in a corn plant genetically modified to express a de-epitoped HMW glutenin, the expression of the corresponding non-mutated HMW glutenin polypeptide is down-regulated compared to a wild-type a corn plant. In certain embodiments, the genetically modified corn plant comprises an RNA silencing agent directed towards the non-mutated polypeptide. In some embodiments, the genetically modified corn plant is genetically modified by a DNA editing agent.

In some embodiments, disclosed herein is a flour generated from the wheat genetically modified to express a de-epitoped HMW glutenin. In some embodiments, disclosed herein is a flour generated from the wheat genetically modified to express at least one de-epitoped HMW glutenin. In some embodiments, disclosed herein is a flour generated from the wheat genetically modified to express multiple de-epitoped HMW glutenin polypeptides.

In some embodiments, disclosed herein is a dough generated from a wheat comprising a de-epitoped HMW glutenin. In some embodiments, disclosed herein is a dough generated from a wheat comprising at least one de-epitoped HMW glutenin. In some embodiments, disclosed herein is a dough generated from a wheat comprising multiple de-epitoped HMW glutenin polypeptides.

In certain embodiments, disclosed herein is a dough comprising a flour comprising a de-epitoped HMW glutenin. In certain embodiments, disclosed herein is a dough comprising a flour comprising a de-epitoped HMW glutenin and no WT HMW glutenin polypeptides.

The amount and variety of de-epitoped HMW glutenin polypeptides can be adjusted to change the quality of the flour or the dough generated therefrom. Thus, in some embodiments, use of any of a de-epitoped HMW glutenin polypeptides disclosed herein, or a combination thereof, improves a dough compared with a dough to which a de-epitoped HMW glutenin polypeptide has not been added. A skilled artisan would appreciate that improvement in a dough comprise generating a dough having more similar in properties (e.g. strength, elasticity) to a dough made from wheat flour relative to how a dough made from non-wheat flour behaves (e.g. almond flour, teff flour).

According to still another embodiment, a flour is generated from wheat which has been genetically modified to express at least one de-epitoped HMW glutenin polypeptide as disclosed herein. In some embodiments, the genetically modified wheat has been further manipulated such that expression of wild-type HMW glutenin polypeptides have been down-regulated or eliminated (as described herein). In some embodiments, the wheat of may be used to generate other edible products such as beer.

In certain embodiments, dough is generating from any of the flours described herein.

Accordingly, in some embodiments, the dough strengthening ability is not reduced by more than 50%, 60%, 70%, 80% as compared to the wild-type polypeptide. Bread generated from flour comprising a modified polypeptide is typically at least 50% more elastic than bread generated from flour in which there is no HMW glutenin.

A skilled artisan would appreciate that the term “dough” encompasses the commonly used meaning, namely, a composition comprising as minimal essential ingredients flour and a source of liquid, for example at least water that is subjected to kneading and shaping. The dough is characterized by its malleability.

A skilled artisan would appreciate that the term “malleable” with respect to a dough may encompass the capacity of the dough for adaptive changes without necessarily being easily broken and as such its pliability, elasticity and/or flexibility which thereby allows the subjecting of the dough to any one of the following processing steps: stretching, shaping, extending, sheeting, morphing, fitting, kneading, molding, modeling, or the like. The shaping of the dough may be by any instrument having predetermined shapes or by a rolling pin or by hand.

The dough of some embodiments may comprise additional components such as salt, plant starch, a flavoring agent, vegetable or vegetable part, oil, vitamins and olives.

The dough may further comprise a leavening agent, examples of which include unpasteurized beer, buttermilk, ginger beer, kefir, sourdough starter, yeast, whey protein concentrate, yogurt, biological leaveners, chemical leaveners, baking soda, baking powder, baker's ammonia, potassium bicarbonate and any combination thereof.

In some embodiments, a dough is combined with at least one additional food ingredient. In some embodiments, a dough is combined with at least one additional food ingredient comprising a flavoring agent, a vegetable, a vegetable part, a mix of vegetables, or a mix of vegetable parts, an oil, a plant starch, a vitamin, vitamins, or olives.

Processed products generated from the doughs comprising a de-epitoped glutenin described herein include, but are not limited to pan bread, a pizza bread crust, a pasta, a tortilla, a Panini bread, a pretzel, a pie and a sandwich bread product.

In some embodiments for a processed dough product prepared by processing a dough comprising a de-epitoped HMW glutenin, the processing step comprises combining the dough with a food ingredient, rising, kneading, extruding, molding, shaping, cooking, stewing, boiling, broiling, baking, frying and any combination of same.

In some embodiments for a processed dough product prepared by processing the dough comprising a de-epitoped HMW glutenin, the processing is in a form comprising a pan bread, a pizza bread crust, a pasta, a tortilla, a Panini bread, a pretzel, a pie and a sandwich bread product.

In some embodiments, disclosed herein is a method of producing flour comprising processing the wheat comprising a de-epitoped HMW glutenin as described herein, thereby producing the flour. In some embodiments, said processing comprises grinding or milling.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of de-epitoped HMW glutenin, methods producing same, and method of de-epitoping HMW glutenin are presented in a range format, for example the number of antigenic units mutated or the number of amino acids mutated within an antigenic unit. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

In some embodiments, the term “about”, refers to a deviance of between 0.0001-5% from the indicated number or range of numbers. In some embodiment, the term “about”, refers to a deviance of between 1-10% from the indicated number or range of numbers. In some embodiments, the term “about”, refers to a deviance of up to 25% from the indicated number or range of numbers.

### EXAMPLES

Reference is now made to the following examples, which together with the above descriptions illustrate some embodiments of the de-epitoped high molecular weight (HMW) glutenin peptides, in a non-limiting fashion.

Generally, the nomenclature used herein, and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Son s, Baltimore, Maryland (1989); Perbal, “A Practical Guide to Molecular Cloning”, John Wiley & Son s, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, C T (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Application s”, Academic Press, San Diego, C A (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Materials and Methods

Computational Design of Peptides for Reduced HL A-DQ8 Binding

Close and more distantly related sequence homologs of high molecular weight glutenin subunit (uniprot accessions X12929, SEQ ID NO: 48 and X12928, SEQ ID NO: 47) were identified by searches performed at the protein level, by either a blast search (Altschul, Stephen F., et al. “Gapped BLAST and PSI-BLAST: a new generation of protein database search programs.” Nucleic acids research 25.17 (1997): 3389-3402.or by HHblits, which employs a Hidden Markov Model (HMM)-based iterative sequence search (Remmert, Michael, et al. “HHblits: lightning-fast iterative protein sequence searching by HMM-HMM alignment.” Nature methods 9.2 (2012): 173-175). Sequences with an expected value greater than 10-1 were discarded. Next, sequences were aligned using Clustal Omega (Sievers, Fabian, et al. “Fast, scalable generation of high-quality protein multiple sequence alignments using Clustal Omega.” Molecular systems biology 7.1 (2011): 539), followed by identification and extraction of all sequence segments aligned to the epitopes of interest. The design of peptides predicted to bind HLA-DQ8 with reduced affinity was performed by in-silico structural modeling and energy calculations, via the Maestro graphical interface to the Schrodinger software suite (Schrodinger, L. L. C. “The Maestro suite of programs: A powerful, all-purpose molecular modeling environment.” New York: Schroedinger LLC (2005)). The solved crystal structure of HLA-DQ8 bound to alpha gliadin epitope HLA-DQ8-glia-al (PDB accession 4z7v) was used as a starting structure. The structure of the de-amidated epitope DQ8-glut-H1 (GQEGYYPTSPEQS; SEQ ID NO: 50) was modeled by simple sidechain mutating of the alpha-gliadin peptide structure, using the “mutate residues” module. Structural refinement was performed on the starting structures using the Protein Preparation Wizard. Next, using the Residue Scanning tool, monte-carlo sampling was performed in search of peptide sequences, where conserved mutations were combined and the predicted change in binding energy upon mutation, AAG, was calculated. Candidate peptide sequences were selected for experimental validation based on the predicted change in binding energy (where an increase of at least 10 relative energy units was required), followed by manual inspection of the generated interaction models.

Due to the nature of the assay, it was determined that 15-mer peptides were optimal for assaying affinity (IC50 binding). The peptides were synthesized as purified material on the 2 mg scale with free acid C-termini (A&A labs, USA). One skilled in the art would appreciate that the 15-mers used included shorter de-epitoped high molecular weight glutenin peptide sequences (mutated repeating antigenic units), as described throughout. For example, Table 2 provides a listing of 15-mers and the shorter mutated repeating antigenic units present therein.

TABLE 2
15-mers and the shorter mutated repeating antigenic units
comprised therein.

| 15-mer Peptide | repeating antigenic units |
|---|---|
| QGQQGYYPTSPQQSG- | QGYYPTSPQ- |
| SEQ ID NO: 49 | SEQ ID NO: 1 |
| QGQEGYYPTSPEQSG- | De-amidated version of |
| SEQ ID NO: 50 | SEQ ID NO: 49. |
| QGAAGYYPTSPRQSG- | AAGYYPTSPR- |
| SEQ ID NO: 51 | SEQ ID NO: 120 |
| QGQSGYDPTSPTQSG- | SGYDPTSPT- |
| SEQ ID NO: 52 | SEQ ID NO: 109 |
| QGQPRYYPTSPRQSG- | PRYYPTSPR- |
| SEQ ID NO: 53 | SEQ ID NO: 110 |
| QGQHGYYPTSLHQSG- | HGYYPTSLH- |
| SEQ ID NO: 54 | SEQ ID NO: 111 |
| QGQEGNDPTGPEQSG- | EGNDPTGPE- |
| SEQ ID NO: 55 | SEQ ID NO: 112 |
| QGQEWHYPTSTEQSG- | EWHYPTSTE- |
| SEQ ID NO: 56 | SEQ ID NO: 113 |
| QGQKGYYPTSPHQSG- | KGYYPTSPH- |
| SEQ ID NO: 143 | SEQ ID NO: 114 |
| QGQEGRGLTSPHQSG- | EGRGLTSPH- |
| SEQ ID NO: 144 | SEQ ID NO: 115 |
| QGQEGRGPASPRQSG- | EGRGPASPR- |
| SEQ ID NO: 145 | SEQ ID NO: 116 |
| QGQEGRYPTSPKQSG- | EGRYPTSPK- |
| SEQ ID NO: 146 | SEQ ID NO: 117 |
| QGQPGYYPTSLTQSG- | PGYYPTSLT- |
| SEQ ID NO: 147 | SEQ ID NO: 119 |
| QGQEGRDLTSPKQSG- | EGRDLTSPK- |
| SEQ ID NO: 169 | SEQ ID NO: 118 |
| QGQHGYDPTSLEQSG- | N/A |
| SEQ ID NO: 173 | |
| QGQEGHGPASPKQSG- | N/A |
| SEQ ID NO: 174 | |
| QGQEGHGPASPPQSG- | N/A |
| SEQ ID NO: 175 | |
| QGQEPYGPSSPEQSG- | N/A |
| SEQ ID NO: 176 | |
| QGQEGYGPTSGEQSG- | N/A |
| SEQ ID NO: 177 | |
| QGQPGYYPTSPHQSG- | N/A |
| SEQ ID NO: 178 | |
| QGQSGYYPTSPTQSG- | N/A |
| SEQ ID NO: 179 | |

N/A-Not applicable as they do not have a corresponding 9-mer.

Measurement of Major Histocompatibility Complex (MHC)/Peptide Interactions:

In-vitro testing of the designed modified gluten peptides was performed by a Major histocompatibility complex (MHC II) binding assay (Sidney, John, et al. “Measurement of MHC/peptide interactions by gel filtration or monoclonal antibody capture.” Current protocols in immunology 100.1 (2013): 18-3)

Briefly, competition assays using different concentrations of wild-type (WT) and modified glutenin peptides were conducted by diluting the peptides in NP40 buffer, and incubation for 2-4 days with purified MHC II and a radiolabeled known MHC binding peptide (peptide probe). MHC II molecules were purified by affinity chromatography, and peptides were radiolabeled using the chloramine T method. After an incubation period, the bound and unbound radiolabeled species were separated, and their relative amounts were determined by either size-exclusion gel-filtration chromatography or monoclonal antibody capture of MHC. The percent of bound radioactivity was then determined. For each modified peptide, IC50 values of WT and modified peptide were calculated. The known glutenin peptide epitopes (SEQ ID NOs: 49 and 50, which are known in the art) were analyzed for MHC binding as a positive control both in a deamidated form (SEQ ID NO: 50 deaminated native peptide) or a non-deamidated form (SEQ ID NO: 49). IC50 values greater than that of the native peptide (SEQ ID NO: 49) by over 4-5-fold signify that the binding of the engineered peptide sequence is compromised with respect to that of the native glutenin peptide. Non-binding was defined as IC50≥30,000 nM.

### Results

Table 3 shows the IC50 measured for several peptide variants that were predicted to have compromised binding to MHC II. Values greater than that of the deaminated peptide (SEQ ID NO: 50) mean that the binding of the engineered peptide chain is compromised with respect to the binding of peptides derived from native glutenin. For each peptide, the number of modifications with respect to the WT native peptide (SEQ ID NO: 49) is listed.

TABLE 3

| | | HLA- | HLA- | HLA- | HLA- | |
|---|---|---|---|---|---|---|
| | # of | DQ8 | DQ2.5 | DQ2.2 | DQ7.5 | |
| | modifi- | IC50 | IC50 | IC50 | IC50 | |
| Variant | cations | (nM) | (nM) | (nM) | (nM) | Sequence |
| P1a | native | 14204 | 29766 | NA** | NA | QGQQGYYPTSPQQSG- |
| | | | | | | SEQ ID NO: 49 |
| P1b | deamidated | 3755 | 3286 | NA | NA | QGQEGYYPTSPEQSG- |
| | | | | | | SEQ ID NO: 50 |
| P1c | 3 | >40000* | >40000 | NA | 19900 | QGAAGYYPTSPRQSG- |
| | | | | | | SEQ ID NO: 51 |
| P1d | 3 | >40000 | >40000 | >40000 | >40000 | QGQSGYDPTSPTQSG- |
| | | | | | | SEQ ID NO: 52 |
| P1e | 3 | 32509 | 33171 | NA | 19400 | QGQPRYYPTSPRQSG- |
| | | | | | | SEQ ID NO: 53 |
| P1f | 3 | >40000 | >40000 | >40000 | >40000 | QGQHGYYPTSLHQSG- |
| | | | | | | SEQ ID NO: 54 |
| P1g | 5 | >40000 | >40000 | >40000 | >40000 | QGQEGNDPTGPEQSG- |
| | | | | | | SEQ ID NO: 55 |
| P1h | 5 | >40000 | >40000 | 5724 | >40000 | QGQEWHYPTSTEQSG- |
| | | | | | | SEQ ID NO: 56 |

*>40000 signifies binding is undetectable by assay
**NA-binding was not measured.

These results demonstrate that introducing 3 or more mutations into the antigenic epitope reduces the binding affinity to MHC II up to a level where binding is indetectable (See for example, the results of SEQ TD NOs: 52, 54, 55, and 56). Variants Plc and Pie do show a significant reduction in binding affinity to MHC II, bringing it to levels that are similar to the non-deamidated peptide, to levels which may still activate T-cells, only to a lower extent than the deamidated peptide. Variant P1h displays HLA-DQ2.2 binding affinity at levels that are similar to those of a deamidated peptide.

Table 4 shows the (C50 measured for addition al variants that were predicted to have compromised binding to MHCII. IC50 values greater than that of the deaminated peptide by 4-5 fold, as shown in Table 2, mean that the binding of the engineered peptide chain is compromised with respect to binding of peptides derived from native glutenin. Underlined amino acids represent residues that have been modified (substituted) compared with the native peptide presented in SEQ TD NO: 49.

TABLE 4

| | HLA-DQ2.5 | HLA-DQ8 | HLA-DQ2.2 | HLA-DQ7.5 |
|---|---|---|---|---|
| Sequence | IC50 (nM) | IC50 (nM) | IC50 (nM) | IC50 (nM) |
| QGQKGYYPTSPHQSG- | 34445 | >40000* | NA ** | NA |
| SEQ ID NO: 143 | | | | |
| QGQEGRGLTSPHQSG- | >40000 | >40000 | >40000 | 39284 |
| SEQ ID NO: 144 | | | | |
| QGQEGRGPASPRQSG- | >40000 | >40000 | >40000 | >40000 |
| SEQ ID NO: 145 | | | | |
| QGQEGRYPTSPKQSG- | >40000 | >40000 | >40000 | >40000 |
| SEQ ID NO: 146 | | | | |
| QGQPGYYPTSLTQSG- | >40000 | >40000 | >40000 | >40000 |
| SEQ ID NO: 147 | | | | |
| QGQEGRDLTSPKQSG- | >40000 | >40000 | >40000 | >40000 |
| SEQ ID NO: 169 | | | | |
| QGQHGYDPTSLEQSG- | 15928 | >40000 | NA | NA |
| SEQ ID NO: 173 | | | | |
| QGQEGHGPASPKQSG- | >40000 | >40000 | >40000 | 16409 |
| SEQ ID NO: 174 | | | | |
| QGQEGHGPASPPQSG- | 22107 | >40000 | NA | NA |
| SEQ ID NO: 175 | | | | |
| QGQEPYGPSSPEQSG- | >40000 | >40000 | >40000 | 28615 |
| SEQ ID NO: 176 | | | | |
| QGQEGYGPTSGEQSG- | 25278 | >40000 | NA | NA |
| SEQ ID NO: 177 | | | | |
| QGQPGYYPTSPHQSG- | 29373 | >40000 | NA | NA |
| SEQ ID NO: 178 | | | | |
| QGQSGYYPTSPTQSG- | 29668 | >40000 | NA | NA |
| SEQ ID NO: 179 | | | | |

*IC50 value higher than assay detection
**NA-binding was not measured.

### Example 2

### Exemplary High Molecular Weight Glutenin Peptides and Polypeptides Showing Abolishment of T Cell Activation

Fractionation of Gluten Proteins from Wheat Flour

High molecular weight glutenins were prepared from wheat flour as described by (Schalk, Kathrin, et al. “Isolation and characterization of gluten protein types from wheat, rye, barley and oats for use as reference materials.” PloS one 12.2 (2017): e0172819.). Briefly, flour was defatted using n-pentane/ethanol solution. Defatted flour was extracted with salt solution. The sediment was extracted with ethanol/water and the resulting supernatant was concentrated and dialyzed against 0.01M acetic acid and lyophilized (proalmin fraction). The remaining sediment was extracted with 2-propanol/water (50/50, v/v)/0.1 mol/l Tris-HCl, pH 7.5, containing 2 mol/L (w/v)urea and 0.06 mol/L (w/v) dithiothreitol (DTT)) for 30 min at 60° C. Following centrifugation, the supernatant was collected, and acetone was added to a final concentration of 40% (V/V). The mixture was allowed to sit at room temperature for 10 minutes and then centrifuged to precipitate the High molecular weight glutenin (HMW) fraction.

Purification of Recombinant HMW from E. coli for T Cell Assay

Bacterial pellet expressing HMW protein was resuspended in buffer A (50 mM Tris pH 9, 50 mM NaCl) and cells were lysed using sonication. Cell lysate was centrifuged and pellet was washed with buffer A. Pellet was solubilized with extraction buffer (8M Urea, 10 mM DTT, 100 mM acetic acid) and incubated at 65° C. for 1 hour with vortex every 10 mins. Sample was centrifuged and supernatant was transferred into a new bottle. DDW was added to supernatant at 1:1 ratio and incubated for 15 minutes at room temperature. Sample was centrifuged and supernatant was transferred to a new bottle. Acetone was added up to 80% and incubated for 18 hours at 4° C. Approximately 80% of the supernatant was discarded and the remaining volume was centrifuged to form a pellet containing HMW protein

Pepsin Chemotrypsin Digestion of HMW for T Cell Assay

200 mg of protein were first incubated in 5% formic acid with 4 mg pepsin for 4 hours in 37° C. Then the sample was evaporated using speedVac. On the following day, the protein was incubated with 4 mg chymotrypsin in 20 mM ammonium-bicarbonate for 4 hours in 37° C., followed by speedVac evaporation. The concentration of the digest was then measured, and the digest was deamidated using transglutaminase, 2 U per 7 mg digest for 6 hours in 37° C. The samples were then cleaned using a C18 column, dried and resuspended in media at a concentration of 2 mg/ml.

Biopsy Processing for T-Cell Lines Generation

Gluten-reactive T-cell lines (TCLs) were generated based on a previously described method, with modifications (Gianfrani, Carmen, et al. “Transamidation of wheat flour inhibits the response to gliadin of intestinal T cells in celiac disease.” Gastroenterology 133.3 (2007): 780-789 Briefly, mucosal explants were digested with collagenase A and cells were seeded at 2-3×10⁵ cells/ml in complete medium X-Vivo15 (Lonza) supplemented with 5% AB-pooled human serum (Biotag) and antibiotics. Cells were stimulated with 1.5×10⁶ irradiated PBMC and TG2 (Sigma-Aldrich)-treated (deamidated) pepsin chemotrypsin (PCT-) digested HMW (50 μg/ml). IL-15 and IL-2 (Peprotech) were added after 24 h at 10 ng/ml and 20 units/ml respectively. Cytokines were supplemented every 3-4 days and cells were split according to need. The cells were restimulated approximately 2 weeks after the first stimulation.

T-Cell Activation Assay

TCLs from celiac disease patients' biopsies were assayed for responses to deamidated PCT-HMW proteins and HMW peptides by the detection of IFN-7 by enzyme-linked immunosorbent assay (ELISA), as previously described (Gianfrani, Carmen, et al. “Gliadin-specific type 1 regulatory T cells from the intestinal mucosa of treated celiac patients inhibit pathogenic T cells.” The journal of immunology 177.6 (2006): 4178-4186.). HLA-matched B-LCLs (Sigma-Aldrich) or orthologous PBMCs were used as antigen presenting cells (APCs). PCT-HMW proteins (100 μg/ml) or peptides (10 μM) (A&A labs) were added to APCs (1×10⁵) concomitantly with responder T cells (4×10⁴). The cells were seeded in 200 μl X-vivo15 medium in round-bottom 96 well plate (Corning) and incubated for 48 h. Each peptide/protein was tested in 4 replicates. DMSO serves as negative control for peptides testing and blank medium serves as negative control for protein testing. For ELISA experiments, Nunc MaxiSorp plates (Thermo Fisher) were coated with 1 μg/ml α-IFNγ antibody (Mabtech), blocked and incubated overnight with 50 μl of the sups taken from the TCLs' plates. Recombinant IFNγ (Bactlab) was used for standard curve generation. The plate was incubated with biotin-α-IFNγ antibody (1 μg/ml) (Mabtech), streptavidin-HRP (Bactlab)(1:5000) and TMB (Thermo Fisher). The reaction was stopped, and the plate was read on the ELISA plate reader at 450 nM. The results were analyzed using Graphpad Prism and IFNγ levels were determined. The results were normalized to the control. Results are considered positive (activating T cells) if IFNγ levels are ≥2-fold in peptide/protein samples compared to control and if IFNγ levels are significantly higher than the control (one-sided student t-test).

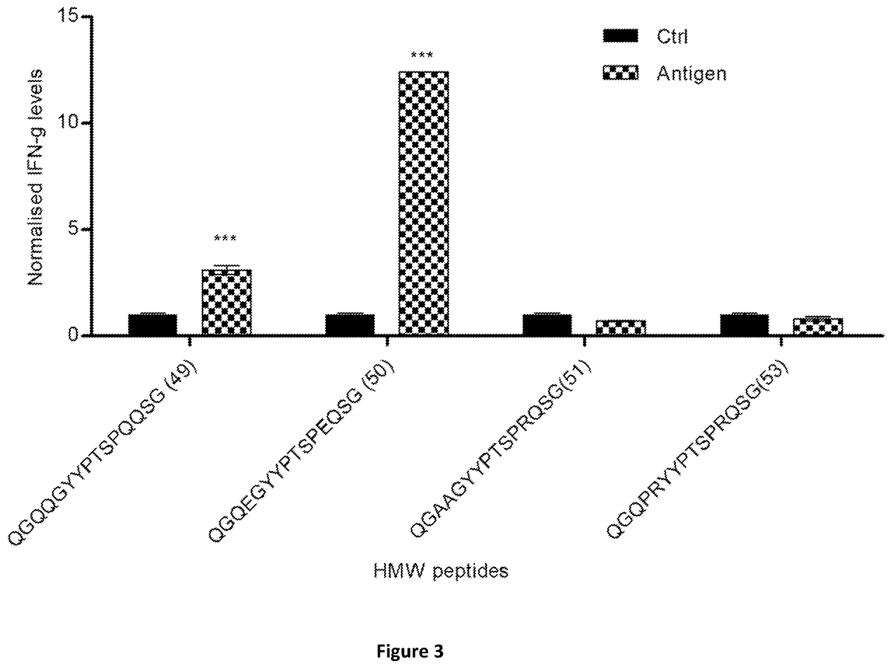

The results are illustrated in FIGS. 3-9.

FIG. 3 shows modifications to HMW-GS immunogenic peptide (modified peptides are SEQ ID NOs: 51 and 53) lead to abolishment of T-cell activation. Mean response to tested HMW-GS WT and modified peptides of TCLs from one CD patient biopsy was assayed by an ELISA detecting levels of IFN-γ. Data shown as mean±SD of four experiments performed for each sample. The TCL response to HMW-GS was considered positive when normalized IFN-γ production was significantly higher for a tested peptide compared to control (as determined by a one-sided student's T-test. * p-val<0.05; **p<0.01; ***p<0.001).

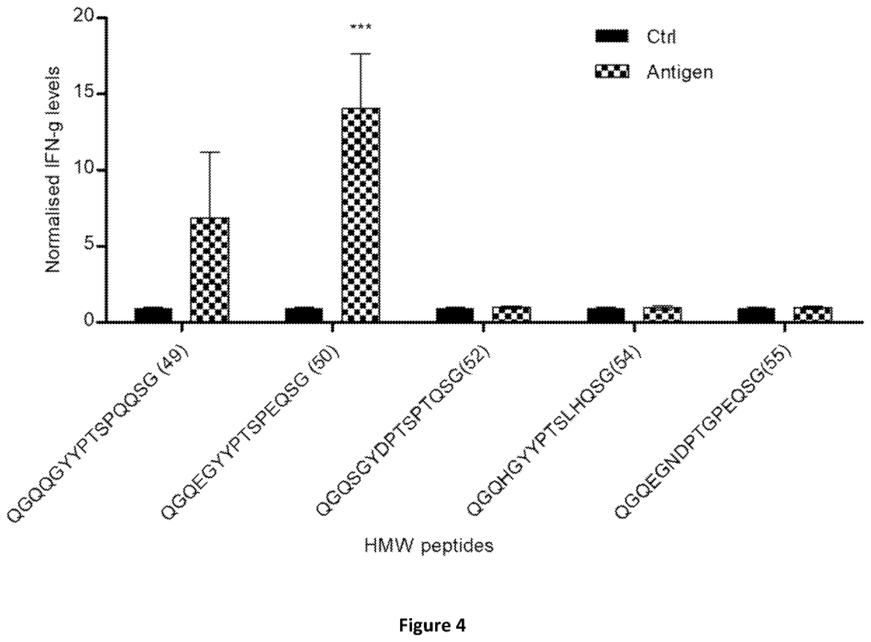

FIG. 4 shows modifications to HMW-GS immunogenic peptide (modified peptides are SEQ ID NOs: 52, 54, and 55) lead to abolishment of T-cell activation. Mean response to tested HMW-GS WT and modified peptides of TCLs from sixteen patient biopsies was assayed by an ELISA detecting levels of IFN-γ. Data shown as mean±SD of four experiments performed for each sample. The TCL response to HMW-GS was considered positive when normalized IFN-γ production was significantly higher for a tested peptide compared to control (as determined by a one-sided student's T-test. * p-val<0.05; **p<0.01; ***p<0.001).

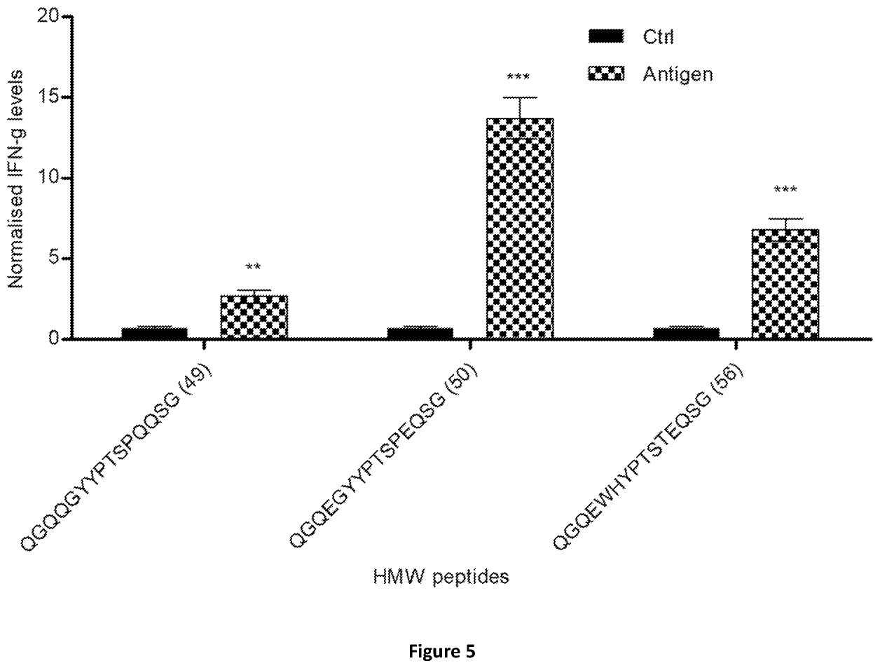

FIG. 5 shows modifications to HMW-GS immunogenic peptide (modified peptide is SEQ ID NO: 56) lead to abolishment of T-cell activation. Mean response to tested HMW-GS WT and modified peptides of TCLs from one CD patient biopsy was assayed by an ELISA detecting levels of IFN-γ. Data shown as mean±SD of four experiments performed for each sample. The TCL response to HMW-GS was considered positive when normalized IFN-γ production was significantly higher for a tested peptide compared to control (as determined by a one-sided student's T-test. * p-val<0.05; **p<0.01; ***p<0.001).

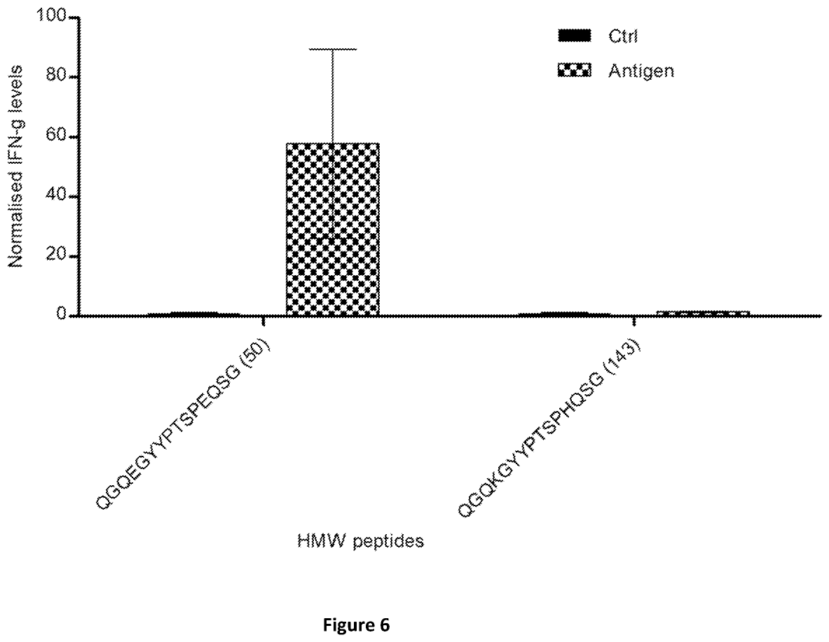

FIG. 6 shows modifications to HMW-GS immunogenic peptide (modified peptide is SEQ ID NO: 143) lead to abolishment of T-cell activation. Mean response to tested HMW-GS WT and modified peptides of TCLs from one CD patient biopsy was assayed by an ELISA detecting levels of IFN-γ. Data shown as mean±SD of four experiments performed for each sample. The TCL response to HMW-GS was considered positive when normalized IFN-7 production was significantly higher for a tested peptide compared to control (as determined by a one-sided student's T-test. * p-val<0.05; **p<0.01; ***p<0.001).

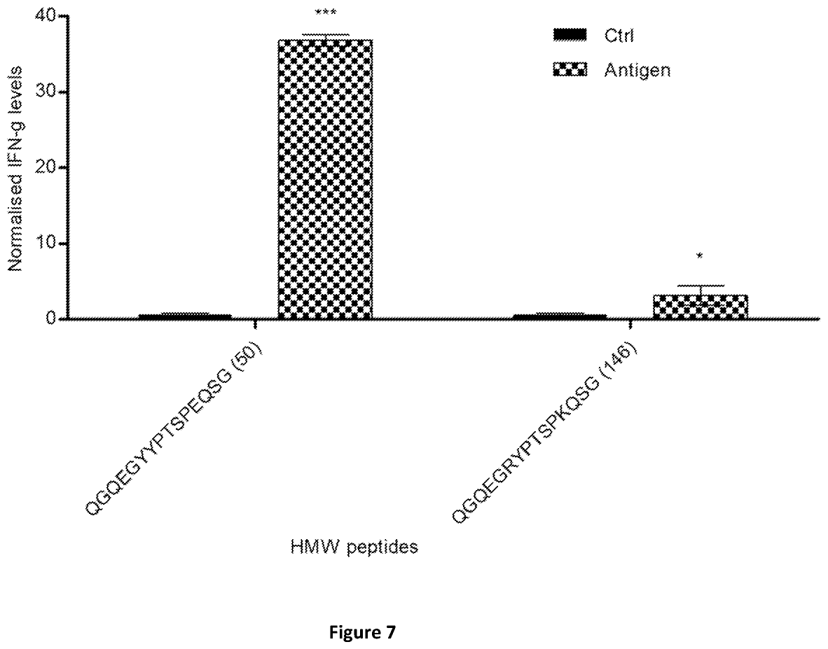

FIG. 7 shows modifications to HMW-GS immunogenic peptide (modified peptide is SEQ ID NO: 146) lead to abolishment of T-cell activation. Mean response to tested HMW-GS WT and modified peptides of TCLs from one CD patient biopsy was assayed by an ELISA detecting levels of IFN-γ. Data shown as mean±SD of four experiments performed for each sample. The TCL response to HMW-GS was considered positive when normalized IFN-7 production was significantly higher for a tested peptide compared to control (as determined by a one-sided student's T-test. * p-val<0.05; **p<0.01; ***p<0.001)

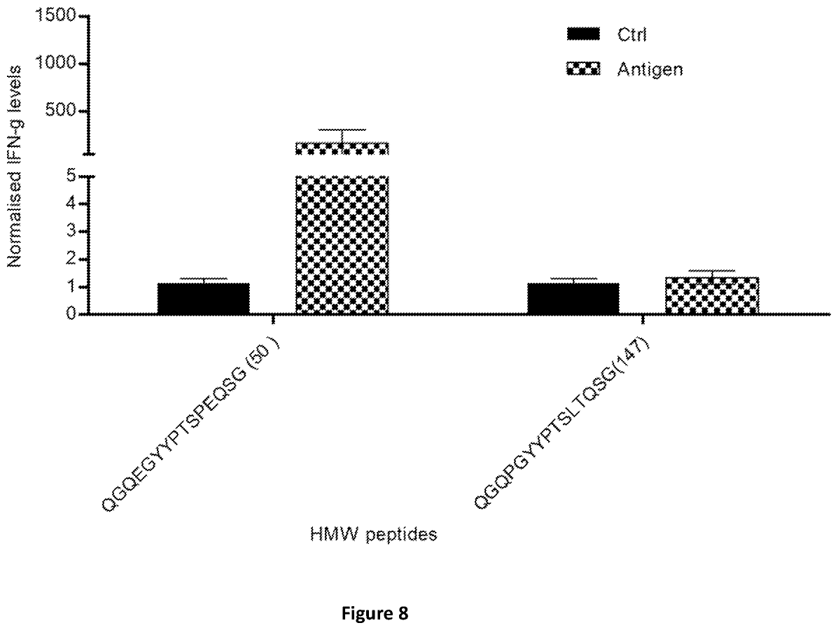

FIG. 8 shows modifications to HMW-GS immunogenic peptide (modified peptide is SEQ ID NO: 147) lead to abolishment of T-cell activation. Mean response to tested HMW-GS WT and modified peptides of TCLs from eight patient biopsies was assayed by an ELISA detecting levels of IFN-7. Data shown as mean±SD of four experiments performed for each sample. The TCL response to HMW-GS was considered positive when normalized IFN-7 production was significantly higher for a tested peptide compared to control (as determined by a one-sided student's T-test. * p-val<0.05; **p<0.01; ***p<0.001).

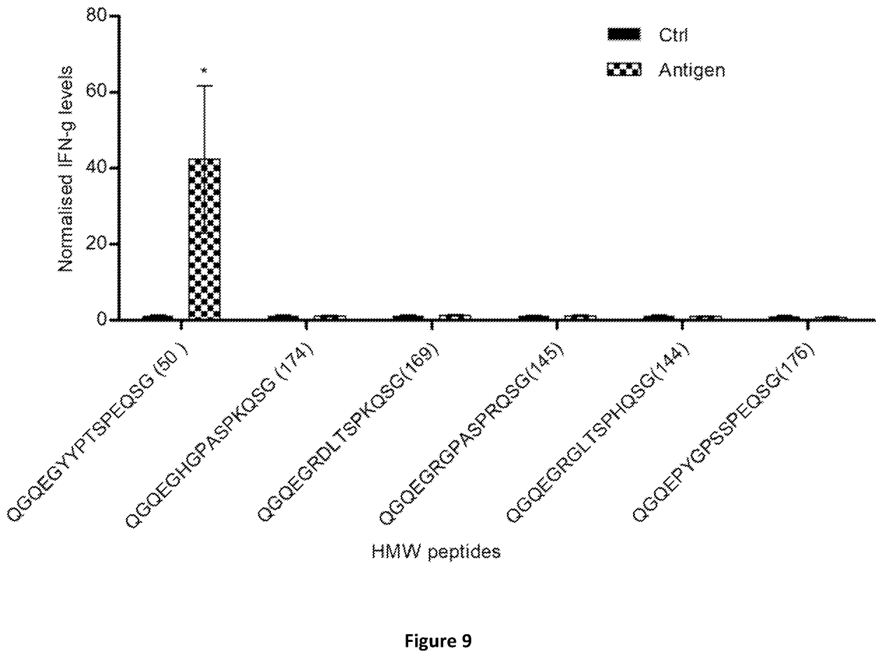

FIG. 9 shows modifications to HMW-GS immunogenic peptide (modified peptides are SEQ ID NOs: 174, 169, 145, 144, and 176) lead to abolishment of T-cell activation. Mean response to tested HMW-GS WT and modified peptides of TCLs from sixteen patient biopsies was assayed by an ELISA detecting levels of IFN-7. Data shown as mean±SD of four experiments performed for each sample. The TCL response to HMW-GS was considered positive when normalized IFN-7 production was significantly higher for a tested peptide compared to control (as determined by a one-sided student's T-test. * p-val<0.05; **p<0.01; ***p<0.001)

Summary: The results demonstrate the modification of validated glutenin peptide epitopes results in a decreased T-cell activation (reduced immunogenicity) compared with the native and deamidated native peptides.

### Example 3

Evaluation of Specific IgE from Wheat Allergic Patient Sera to Wheat Extract and Wild-Type (WT) and De-Epitoped (DE) HMW Glutenin Recombinant Proteins

Objective: To evaluate the allergenicity of recombinant HMW glutenin proteins (Wild-Type —WT and de-epitoped variants—DE).

Methods:

Protein Purification

Recombinant bacteria were used to express HMW glutenin WT and variant proteins for purification. HMW glutenin WT Dx5 (SEQ ID NO: 47) and representative de-epitoped HMW glutenin variants (SEQ ID Nos: 102, 128) were purified by the following procedure: Bacterial cell pellets expressing HMW glutenin proteins were resuspended in a lysis buffer, containing sucrose, TRIS, MgCl₂ lysozyme. The cells were lysed using pressure homogenizer, followed by the addition of DNAse and EDTA. Cell lysate was centrifuged, and the pellet was washed with the a wash buffer, containing TRIS and Tween20 and centrifuged. Pellet was washed again with 0.1 mM acetic acid buffer and centrifuged to collect the pellet containing the HMW protein.

HMW glutenin WT Dy10 (SEQ ID NO: 48) and representative de-epitoped Dy10 (SEQ ID NO: 169) variant protein were produced using the same procedure as that described in Example 2 (Purification of recombinant HMW from E. coli for T cell assay)

Standard ELISA Protocol

For 384-well ELISA plate coating with antigens, wheat extract (WE), recombinant HMW glutenin (rHMW) Dx5 WT protein (SEQ ID NO: 47), de-epitoped (DE) rHMW glutenin Dx5 variant (SEQ ID NOs: 102, 128), rHMW Dy10 WT glutenin protein (SEQ ID NO: 48), rHMW DE Dy10 glutenin variant (SEQ ID NO: 169), all solubilized in 8M urea+10 mM DTT, were diluted 1:200 in carbonate-bicarbonate coating buffer and used for well coating. For the BSA-coated wells, blocking buffer (PBS+0.5% BSA) was used for coating. Each coated antigen was coated in triplicates for each tested sample. The plate was incubated overnight in 4° C. on an orbital shaker, 120 rpm. The coating solution was discarded, and wells were blocked with blocking buffer, for 1 hour on orbital shaker, 120 rpm, at RT. Samples (patient sera and plasma), including a negative control (serum from an individual not allergic to wheat), were diluted 1:12 in dilution buffer (PBST+0.5% BSA). Anti-HMW glutenin mouse pAb was diluted 1:1000 in dilution buffer. Blocking buffer was discarded, diluted samples were added to the wells and incubated overnight in 4° C. on an orbital shaker, 120 rpm. Sera was discarded, plate was washed with PBST and secondary Ab was added (anti mouse IgG-HRP for mouse anti HMW glutenin pAb wells or anti human IgE-HRP for the other samples, diluted 1:10000 in dilution buffer). Plate was incubated for 30 minutes on an orbital shaker, 120 rpm, at RT, HRP-conjugated Ab solution was discarded, and plate was washed with PBST. Signal was developed using TMB substrate.

For data analysis, average values for each triplicate were calculated. For each sample average value, [(S−N)/N] value was determined as follows: S—the tested average value, N—negative control average value. For each sample, [(S−N)/N] values against all antigens were plotted, and 1-way Anova was used to determine significance compared to WE:

| | |
|---|---|
| Ns | P > 0.05 |
| * | P ≤ 0.05 |
| ** | P ≤ 0.01 |
| *** | P ≤ 0.001 |

Results:

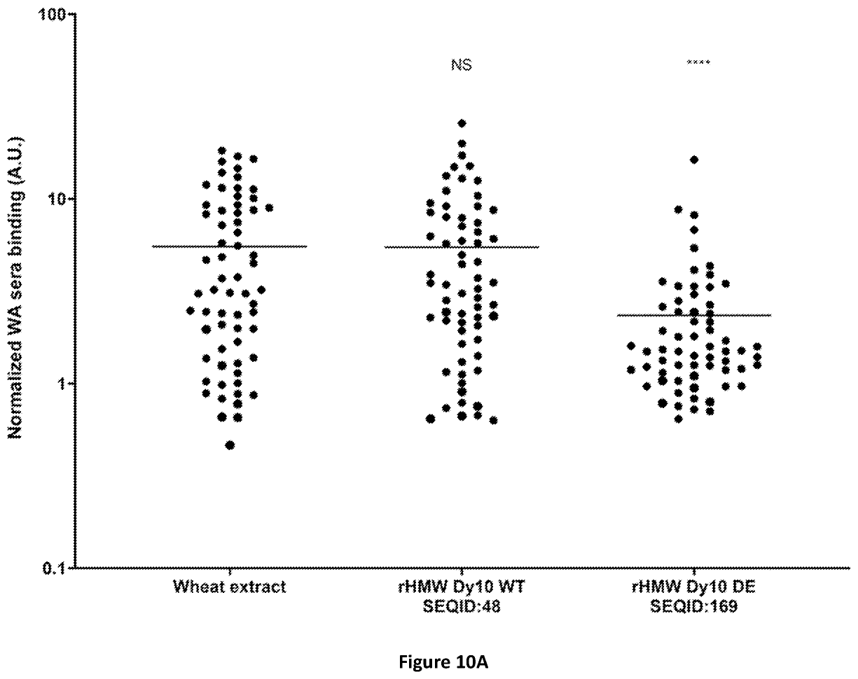

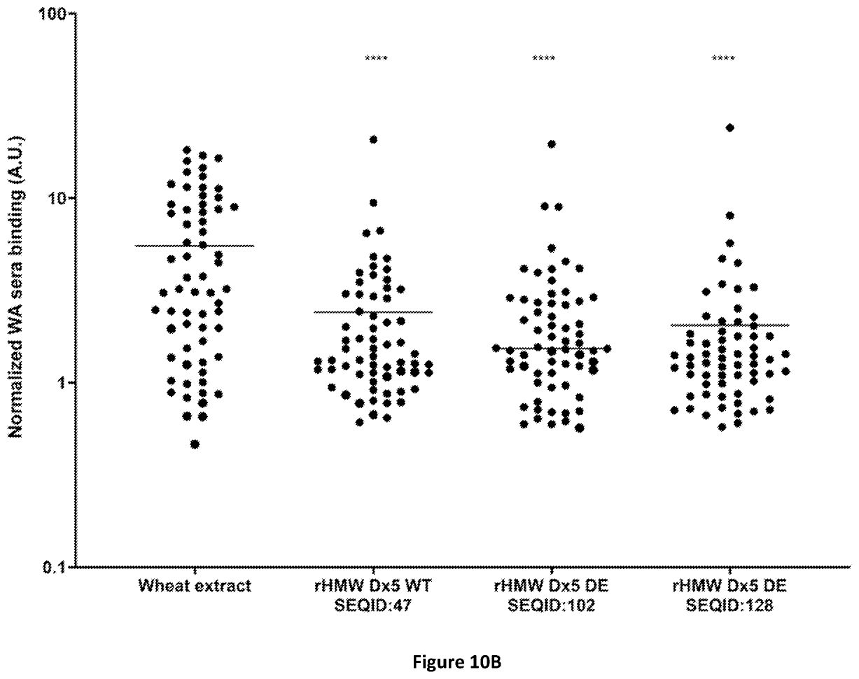

WT Dy10 demonstrates overall similar binding to wheat allergy IgE sera compared to wheat extract, whereas the DE variant displays a significant reduction in IgE binding (FIG. 10A). WT Dx5 and the DE variants demonstrate statistically lower binding to wheat allergy IgE sera compared with wheat extract (FIG. 10B).

Analysis of the WT and DE variants in an ex-vivo assay testing degranulation of basophiles in response to the WT and modified DE proteins, will identify WT or DE variants with reduced allergenicity. A reduced response compared with the response of a wheat extract correlates with WT or DE variants with reduced allergenicity.

### SUMMARY

A food product that does not contain any gliadins and has HMW glutenin with potentially reduced allergenicity (recombinant WT or recombinant DE), may be helpful for human subject suffering from non-severe allergy to HMW glutenins.

While certain features of the de-epitoped high molecular weight (HMW) glutenin polypeptides, and methods of production and use thereof, have been illustrated and described herein, many modifications, substitutions, changes, and equivalents will now occur to those of ordinary skill in the art. It is, therefore, to be understood that the appended claims are intended to cover all such modification s and changes as fall within the true spirit of the de-epitoped high molecular weight (HMW) glutenin polypeptides.

1.-43. (canceled)

44. A de-epitoped high molecular weight (HMW) glutenin comprising repeating antigenic units, wherein at least one repeating antigenic unit comprises a first mutation at position 1 and a second mutation at position 9 of said repeating antigenic unit, wherein the amino acid sequence of a non-mutated repeating antigenic unit is set forth in any of SEQ ID NOs: 1-33, 35, or 71-99, and wherein said first and second mutations each comprise an amino acid substitution mutation or a deletion of an amino acid mutation.

45. The de-epitoped HMW glutenin of claim 44, wherein
(a) said first mutation at position 1 of said antigenic unit is a replacement with a histidine, and said second mutation at position 9 of said antigenic unit is a replacement with histidine; or
(b) said first mutation at position 1 of said antigenic unit is a replacement with a proline, and said second mutation at position 9 of said antigenic unit is a replacement with arginine; or
(c) said first mutation at position 1 of said antigenic unit is a replacement with an alanine, and said second mutation at position 9 of said antigenic unit is a replacement with arginine; or
(d) said first mutation at position 1 of said antigenic unit is a replacement with a serine, and said second mutation at position 9 of said antigenic unit is a replacement with threonine; or
(e) said first mutation at position 1 of said antigenic unit is a replacement with a glutamate, and said second mutation at position 9 of said antigenic unit is a replacement with glutamate; or
(f) said substituting at position 1 of said antigenic unit is a replacement with a proline, and said substituting at position 9 of said antigenic unit is a replacement with threonine; or
(g) said first mutation at position 1 of said antigenic unit is a replacement with a glutamate, and said second mutation at position 9 of said antigenic unit is a replacement with lysine or an arginine; or
(h) said first mutation at position 1 of said antigenic unit is a replacement with a glutamate or a lysine, and said second mutation at position 9 of said antigenic unit is a replacement with histidine; or
(i) said first mutation, or said second mutation, or both comprising a deletion of the amino acids at positions 1-9 of said repeating antigenic unit; or
(j) any combination of (a)-(i) when more than one repeating antigenic unit is mutated.

46. The de-epitoped HMW glutenin of claim 44, comprising at least two additional substitution mutations at any of positions 3, 5, or 8, of said at least one repeating antigenic unit, wherein the substitution at position 5 is leucine and the substitution at position 3 is arginine.

47. The de-epitoped HMW glutenin of claim 44, comprising the amino acid sequence set forth in any of SEQ ID NOs: 52, 102-107, 109-112, 114-116, 118-119, 126-134, 137-138, 140-142, 148-151, 155-168, or 170-172.

48. An isolated polynucleotide encoding the de-epitoped HMW glutenin of claim 44.

49. An expression vector comprising the isolated polynucleotide of claim 48, operatively linked to a transcriptional regulatory sequence so as to allow expression of said de-epitoped high molecular weight glutenin in a cell.

50. A cell comprising the de-epitoped HMW glutenin of claim 44.

51. A method of producing a de-epitoped HMW glutenin, said method comprising
(a) culturing cells that comprise the expression vector of claim 48 under conditions allowing for expression of said de-epitoped HMW glutenin in said cells;
(b) expressing said de-epitoped HMW glutenin; and
(c) collecting said expressed de-epitoped HMW glutenin.

52. A method of de-epitoping a HMW glutenin comprising repeating antigenic units, wherein said method comprises mutating the amino acid residue at position 1 and 9 of at least one repeating antigenic unit of the HMW glutenin, said non-mutated repeating antigenic unit having an amino acid sequence selected from the group consisting of SEQ ID NOs: 1-33, 35, or 71-99, wherein said mutations at positions 1 and 9 each comprise an amino acid substitution mutation or a deletion of an amino acid mutation.

53. The method of claim 52, wherein said substitution mutation at position 9 of said repeating antigenic unit comprises a replacement with
(a) a positively charged amino acid selected from arginine, histidine or lysine;
(b) a small amino acid selected from serine or threonine;
(c) a glutamate; or
(d) a combination thereof when more than one repeating antigenic unit is mutated.

54. The method of claim 52, wherein:
(a) said substituting at position 1 of said antigenic unit is a replacement with histidine, and said substituting at position 9 of said antigenic unit is a replacement with histidine; or
(b) said substituting at position 1 of said antigenic unit is a replacement with proline, and said substituting at position 9 of said antigenic unit is a replacement with arginine; or
(c) said substituting at position 1 of said antigenic unit is a replacement with alanine, and said substituting at position 9 of said antigenic unit is a replacement with arginine; or
(d) said substituting at position 1 of said antigenic unit is a replacement with serine, and said substituting at position 9 of said antigenic unit is a replacement with threonine; or
(e) said substituting at position 1 of said antigenic unit is a replacement with glutamate, and said substituting at position 9 of said antigenic unit is a replacement with glutamate; or
(f) said substituting at position 1 of said antigenic unit is a replacement with proline, and said substituting at position 9 of said antigenic unit is a replacement with threonine; or
(g) said substituting at position 1 of said antigenic unit is a replacement with a glutamate, and said substituting at position 9 of said antigenic unit is a replacement with lysine or an arginine; or
(h) said substituting at position 1 of said antigenic unit is a replacement with a glutamate or a lysine, and said substituting at position 9 of said antigenic unit is a replacement with histidine; or
(i) any combination of (a)-(h) when more than one repeating antigenic unit is mutated.

55. The method of claim 52, further comprising mutating at least two amino acids at positions 3, 5, or 8 of said repeating antigenic unit.

56. The method of claim 55, wherein said substituting at position 5 is replacing the amino acid with a leucine and said substituting at position 3 is replacing the amino acid with an arginine.

57. The method of claim 52, wherein said method further comprises deleting the amino acids at positions 1-9 of the repeating antigenic unit.

58. The method of claim 52, wherein the de-epitoped HMW glutenin comprises the amino acid sequence set forth in any of SEQ ID NOs: 52, 102-107, 109-112, 114-116, 118-119, 126-134, 137-138, 140-142, 148-151, 155-168, or 170-172.

59. A flour comprising the de-epitoped HMW glutenin of claim 44.

60. A modified wheat expressing the de-epitoped HMW glutenin of claim 44.

61. A flour derived from the wheat of claim 60.

62. A dough comprising the flour of claim 44.

63. A modified corn plant expressing the de-epitoped HMW glutenin of claim 44.
